(19) United States
(12) Patent Application Publication
(10) Pub. No.: US 2026/0315406 A1
(43) Pub. Date: Oct. 15, 2026

(54) A COMPOUND FOR PREPARING LIPID NANOPARTICLES ENCAPSULATING AN AGENT, NANOPARTICLE COMPOSITION COMPRISING SAID COMPOUND AND RELATED METHODS THEREOF

(71) Applicant: AGENCY FOR SCIENCE, TECHNOLOGY AND RESEARCH, Singapore (SG)

(72) Inventors: Yi Yan YANG, Singapore (SG); Jinyue ZENG, Singapore (SG); Jin Hao GOH, Singapore (SG); Yue WAN, Singapore (SG)

(73) Assignee: AGENCY FOR SCIENCE, TECHNOLOGY AND RESEARCH, Singapore (SG)

(21) Appl. No.: 19/481,806

(22) PCT Filed: May 10, 2024
(86) PCT No.: PCT/SG2024/050304

§ 371 Date: Nov. 5, 2025

(30) Foreign Application Priority Data
May 11, 2023 (SG) 10202301316R

(51) Int. Cl. A61K 9/51; A61K 39/00; A61K 39/215; A61P 37/04

(52) U.S. Cl. CPC A61K 9/5169; A61K 9/5123; A61K 39/215; A61P 37/04; A61K 2039/53; A61K 2039/55555

(57) ABSTRACT

There is provided a compound represented by general formula (1) for preparing lipid nanoparticles encapsulating a therapeutic, prophylactic and/or biological agent: wherein A comprises a hydrophilic moiety selected from carbohydrate/sugar/saccharide and derivatives thereof; X¹ is —OR^{a} or —NR^{b}R^{c}; X⁴ is —OR^{y}, —SR^{z} or a hydrophilic group, where R^{y} to R^{z} are independently selected from the group consisting of H, optionally substituted alkyl, optionally substituted alkenyl or optionally substituted alkynyl; X² and X³ are each independently —O— or —NR⁶—, where R^{a} to R^{d} are independently selected from the group consisting of H, optionally substituted alkyl, optionally substituted alkenyl or optionally substituted alkynyl; R¹ and R² are each independently a hydrophobic group; R³, R⁴, R⁵, R⁶ and R¹⁰ are each independently H, optionally substituted alkyl, optionally substituted alkenyl or optionally substituted alkynyl; R⁷, R⁹, R¹¹ and R¹² are each independently optionally substituted alkyl, optionally substituted alkenyl or optionally substituted alkynyl; R¹³ is H, optionally substituted alkyl, optionally substituted alkenyl or optionally substituted alkynyl, or —C(═O)R¹⁴, where R¹⁴ is optionally substituted alkyl, optionally substituted alkenyl, optionally substituted alkynyl; w≥1; x is 0 or ≥1; y is 0 or ≥1; and z is 0 or ≥1.

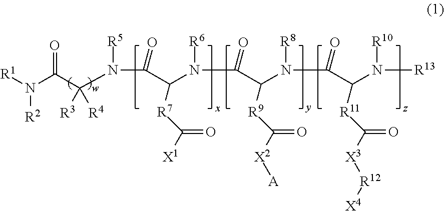

### TECHNICAL FIELD

The present disclosure relates broadly to a compound for preparing lipid nanoparticles encapsulating an agent and a method of preparing said compound. The present disclosure also relates to a nanoparticle composition comprising said compound and related methods and uses.

### BACKGROUND

Lipid nanoparticles are widely used in the delivery of therapeutic, prophylactic and/or biological agents (e.g., polynucleotides such as mRNA). However, a safe, stable and efficacious delivery system remains a challenge. Particularly, there have been reports of adverse health effects and cytotoxicity associated with the use of lipid nanoparticles for delivery.

Currently, there are only 2 mRNA Covid-19 vaccines from Moderna and Pfizer-BioNtech that have been approved by the United States Food and Drug Administration (US FDA) for human use. Both vaccines utilize SARS-CoV-2 mRNA as the antigen and lipids as the carrier. The lipids consist of 3 different types of lipids (ionizable lipid, PEG-lipid conjugate and helper lipid) and cholesterol. The lipids assemble with the mRNA to form nanoparticles that stimulate the immune cells for prophylactic response against the SARS-CoV-2 virus.

However, the currently available formulations have several disadvantages and drawbacks, and are far from desirable. Firstly, the production of anti-lipid and anti-PEG antibodies was evident with such formulations (due to use/presence of PEG-lipid conjugate), which can result in hypersensitivity and anaphylaxis in some subjects. Polyethylene glycol (PEG) has been reported to be a high-risk allergen found hidden in drug/food items. Binding of PEG to basophils through IgE can cause release of compounds that induce allergies and individuals may develop anaphylactic conditions from PEG present in medications. PEG has also been identified as the cause of accelerated blood clearance (ABC) phenomenon. In addition, the presence of anti-PEG antibodies in the body may reduce plasma half-life of mRNA LNPs and their vaccination efficacy. Furthermore, currently available formulations lack immune cell-targeting ability.

In view of the above, there is a need to address or at least ameliorate the above-mentioned problems. In particular, there is a need to provide a compound and/or nanoparticle composition for a cost efficient, substantially safe and stable, and/or efficacious delivery of therapeutic, prophylactic and/or biological agents.

### SUMMARY

In one aspect, there is provided a compound represented by general formula (1) for preparing lipid nanoparticles encapsulating a therapeutic, prophylactic and/or biological agent:

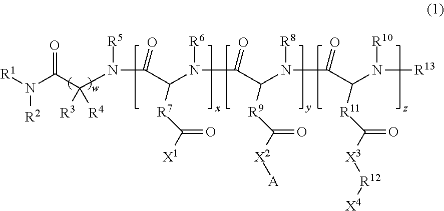

wherein

A comprises a hydrophilic moiety selected from carbohydrate/sugar/saccharide and derivatives thereof;
X¹ is —OR^{a} or —NR^{b}R^{c};
X⁴ is —OR^{y}, —SR^{z} or a hydrophilic group, where R^{y} to R^{z} are independently selected from the group consisting of H, optionally substituted alkyl, optionally substituted alkenyl or optionally substituted alkynyl;
X² and X³ are each independently —O— or —NR^{d}—, where R^{a} to R^{d} are independently selected from the group consisting of H, optionally substituted alkyl, optionally substituted alkenyl or optionally substituted alkynyl;
R¹ and R² are each independently a hydrophobic group;
R³, R⁴, R⁵, R⁶, R⁸ and R¹⁰ are each independently H, optionally substituted alkyl, optionally substituted alkenyl or optionally substituted alkynyl;
R⁷, R⁹, R¹¹ and R¹² are each independently optionally substituted alkyl, optionally substituted alkenyl or optionally substituted alkynyl;
R¹³ is H, optionally substituted alkyl, optionally substituted alkenyl or optionally substituted alkynyl, or —C(═O)R¹⁴, where R¹⁴ is optionally substituted alkyl, optionally substituted alkenyl, optionally substituted alkynyl;
w≥1; x is 0 or ≥1; y is 0 or ≥1; and z is 0 or ≥1.

In one embodiment, the compound is capable of targeting carbohydrate receptors on a cell surface.

In one embodiment, A is represented by general formula (2) having a 6-membered ring structure:

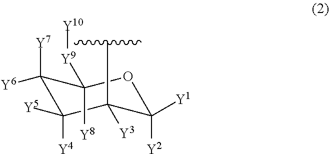

wherein
Y¹ to Y⁸ and Y¹⁰ are each independently selected from —H or —OH; and
Y⁹ is alkyl.

In one embodiment, A is represented by general formula (2A) and/or (28) having a 6-membered ring structure:

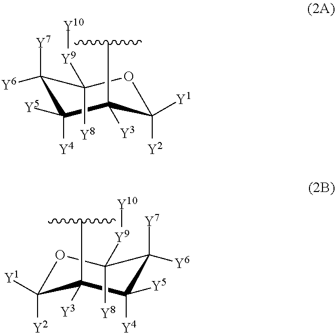

wherein
Y¹ to Y⁸ and Y¹⁰ are each independently selected from —H or —OH; and
Y⁹ is alkyl.

In one embodiment, the total sum of x+y+z is from 1 to 300.

In one embodiment, the hydrophobic group at R¹ and R² each independently comprises optionally substituted alkyl.

In one embodiment, the compound is selected from the group consisting of LPP1, LPP2, LPP3, LPP4, LPP5, LPP6, LPP7, LPP8, LPP9, LPP10, LPP11, LPP12, LPP13, LPP14, LPP15 and combinations thereof.

In another aspect, there is provided a method of preparing a compound as disclosed herein, the method comprising:
(a-i) polymerizing one or more N-carboxyanhydride (NCA) monomers represented by general formula (3) with a lipid initiator represented by general formula (4) to obtain a first intermediate compound represented by general formula (5):

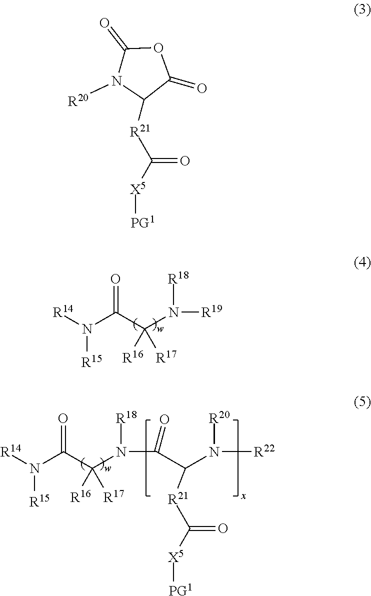

wherein
X⁵ is —O— or —NR^{e}—, where R^{e} is selected from the group consisting of H, optionally substituted alkyl, optionally substituted alkenyl or optionally substituted alkynyl;
R¹⁴ and R¹⁵ are each independently a hydrophobic group;
R¹⁶, R¹⁷, R¹⁸, R¹⁹, R²⁰ and R²² are each independently H, optionally substituted alkyl, optionally substituted alkenyl or optionally substituted alkynyl;
R²¹ is optionally substituted alkyl, optionally substituted alkenyl or optionally substituted alkynyl;
w≥1;
x is 0 or 1; and
PG¹ is a protecting group selected from benzyl, tert-butyl or combinations thereof;
(a-ii) optionally reacting the first intermediate compound represented by general formula (5) with an acylating agent represented by general formula (6) to obtain a second intermediate compound represented by general formula (7):

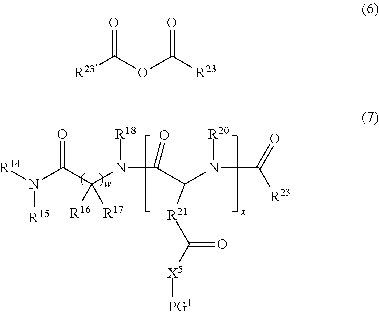

wherein
R²³ and R^{23′} are each independently optionally substituted alkyl, optionally substituted alkenyl or optionally substituted alkynyl;
(a-iii) deprotecting the first intermediate compound represented by general formula (5) and/or the second intermediate compound represented by is general formula (7) to obtain a compound represented by general formula (8);

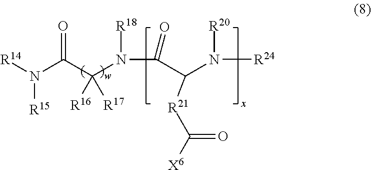

wherein
X⁶ is —OR^{a} or —NR^{f}R^{g}, where R^{a}, R^{f} and R^{g} are independently selected from the group consisting of H, optionally substituted alkyl, optionally substituted alkenyl or optionally substituted alkynyl; and
R²⁴ is H, optionally substituted alkyl, optionally substituted alkenyl or optionally substituted alkynyl, or —C(═O)R²⁵, where R²⁵ is optionally substituted alkyl, optionally substituted alkenyl, optionally substituted alkynyl;
(a-iv) reacting the compound represented by general formula (8) with at least one substrate selected from the following:
   (i) an amidating agent comprising a hydrophilic moiety represented by general formula (9); and
   (ii) an alkanolamine represented by general formula (10) to obtain a compound represented by general formula (11);

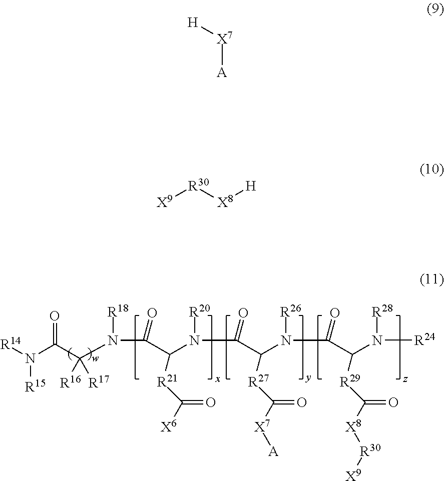

wherein
A comprises a hydrophilic moiety selected from carbohydrate/sugar/saccharide and derivatives thereof;
X⁷ and X⁸ are each independently —O— or —NR^{h}—,
X⁹ is independently —ORⁱ or —NR^{j}R^{k}, where R^{h} to R^{k} are independently selected from the group consisting of H, optionally substituted alkyl, optionally substituted alkenyl or optionally substituted alkynyl;
R²⁶ and R²⁸ are each independently H, optionally substituted alkyl, optionally substituted alkenyl or optionally substituted alkynyl;
R²⁷, R²⁹ and R³⁰ are each independently optionally substituted alkyl, optionally substituted alkenyl or optionally substituted alkynyl;
y is 0 or 1; and
z is 0 or ≥1.

In one embodiment, the method further comprises, prior to step (a-i):
(b-i) reacting a protected amino acid represented by general formula (12) with a carbonylating agent to obtain the N-carboxyanhydride (NCA) monomer represented by general formula (3):

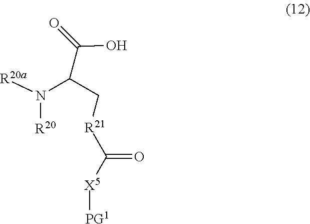

wherein R²0a is H, optionally substituted alkyl, optionally substituted alkenyl or optionally substituted alkynyl.

In one embodiment, the method further comprises, prior to step (a-i):
(c-i) reacting a lipid compound represented by general formula (13) with a protected amino acid represented by general formula (14) to obtain a protected lipid initiator represented by general formula (15):

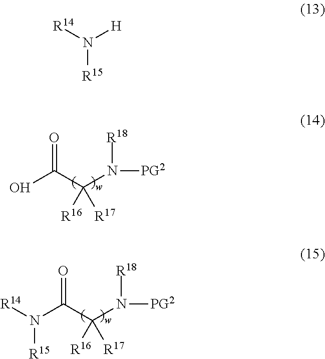

wherein PG² is a protecting group selected from tert-butoxycarbonyl protecting group (BOC), tert-butyl or combinations thereof; and
(c-ii) deprotecting the protected lipid initiator represented by general formula (15) to obtain the lipid initiator represented by general formula (4).

In another aspect, there is provided a nanoparticle composition for delivery of a therapeutic, prophylactic and/or biological agent, the nanoparticle composition comprising:
a compound as disclosed herein; and
a therapeutic, prophylactic and/or biological agent that is encapsulated in said composition.

In one embodiment, the composition further comprises:
(a) ionizable lipid;
(b) helper lipid; and
(c) sterol.

In one embodiment, the ionizable lipid, helper lipid, sterol, and compound represented by general formula (1) are mixed at a mole ratio of 15-140:3-30:14-130:0.2-40.

In one embodiment, the ionizable lipid is selected from ALC-0315, SM-102, Lipid 5, DLinDMA, D-Lin-MC2-DMA, DLin-MC3-DMA, D-Lin-MC4-DMA, Dlin-KC2-DMA, YSK05, AA3-Dlin, SSPaImM, SSPalmO-Phe, Lipid A9, L319, DODMA, CL1, BP Lipid 310, ATX-001, ATX-100, Lipid 2, 80-016B, BP Lipid 309, BP Lipid 307, 93-017S, 93-0170, NT1-014B, 306-012B-3, 306-012B, 113-016B, 3060i10, 3060i9-cis2, BAMEA-016B, Al-28, 113-012B, 98N12-5, Ckk-E12, OF-02, C12-200, BP Lipid 311, BP Lipid 308, BP Lipid 314, BP Lipid 312, LPO1, TCL053, Lipid C24, BP Lipid 315, Lipid 29, 9A1 P9, C13-112-tri-tail, C13-113-tri-tail, C13-112-tetra-tail, or C13-113-tetra-tail, C12-200 and combinations thereof.

In one embodiment, the helper lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin and combinations thereof.

In one embodiment, the sterol is selected from cholesterol, fecosterol, sitosterol, ergosterol, campesterol, stigmasterol, brassicasterol, avenasterol and combinations thereof.

In one embodiment, the nanoparticle composition comprises nanoparticles having a N/P ratio from 2:1 to 40:1.

In one embodiment, the nanoparticle composition comprises nanoparticles having an average particle size of no more than 800 nm.

In one embodiment, the nanoparticle composition comprises nanoparticles having a zeta potential of from −40 mV to +40 mV.

In another aspect, there is provided the nanoparticle composition as disclosed herein for use in medicine.

In another aspect, there is provided the nanoparticle composition as disclosed herein for use in the treatment or prophylaxis of a disease, disorder or condition in a subject in need thereof.

In another aspect, there is provided use of a nanoparticle composition as disclosed herein in the manufacture of a medicament for treatment or prophylaxis of a disease, disorder or condition in a subject in need thereof.

In another aspect, there is provided a method of treating or preventing a disease, disorder or condition in a subject in need thereof, the method comprising administering a therapeutically effective amount of the nanoparticle composition as disclosed herein to the subject.

In one embodiment, an immune response in the subject is to be induced through the administration of the nanoparticle composition thereto.

In one embodiment, the disease, disorder or condition is mediated by a coronavirus.

In one embodiment, the coronavirus is a SARS-CoV-2 coronavirus.

### Definitions

The term “particle” as used herein broadly refers to a discrete entity or a discrete body. The particle described herein can include an organic, an inorganic, a composite particle or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, the term “size” when used in the context of nanoparticle can refer to the diameter of the nanoparticle although it is not limited as such. In various embodiments, when the particle is substantially spherical, the term “size” can refer to the diameter of the particle; or when the particle is substantially non-spherical, the term “size” can refer to the largest length of the particle.

The term “nano” as used herein is to be interpreted broadly to include dimensions in a nanoscale, i.e., less than about 1000 nm, about 1 nm to less than about 1000 nm, about 1 nm to about 900 nm, about 1 nm to about 800 nm, about 1 nm to about 700 nm, about 1 nm to about 600 nm, about 1 nm to about 500 nm, about 1 nm to about 400 nm, about 1 nm to about 300 nm, about 1 nm to about 200 nm, or from about 1 nm to about 100 nm. Accordingly, the term “nanostructures”, “nanoparticles”, “nanomaterials” and the like as used herein may include structures that have at least one dimension in the range of no more than said range. The term “nanostructures”, “nanoparticles”, “nanomaterials” and the like as used herein may include structures that have at least one dimension that is no more than about 1,000 nm, no more than about 950 nm, no more than about 900 nm, no more than about 850 nm, no more than about 800 nm, no more than about 750 nm, no more than about 700 nm, no more than about 650 nm, no more than about 600 nm, no more than about 550 nm, no more than about 500 nm, no more than about 450 nm, no more than about 400 nm, no more than about 350 nm, no more than about 300 nm, no more than about 250 nm, no more than about 200 nm, no more than about 150 nm, no more about 100 nm, no more than about 90 nm, no more than about 80 nm, no more than about 70 nm, no more than about 60 nm, no more than about 50 nm, no more than about 40 nm, no more than about 30 nm, no more than about 20 nm, or no more than about 10 nm.

The term “micro” as used herein is to be interpreted broadly to include dimensions from about 1 micron to about 1000 microns, from about 1 micron to less than about 1000 microns, from about 1 micron to about 900 microns, from about 1 micron to about 800 microns, from about 1 micron to about 700 microns, from about 1 micron to about 600 microns, from about 1 micron to about 500 microns, from about 1 micron to about 400 microns, from about 1 micron to about 300 microns, from about 1 micron to about 200 microns, from about 1 micron to about 100 microns, or from about 1 micron to about 5 microns.

The term “treatment”, “treat” and “therapy”, and synonyms thereof as used herein refer to both therapeutic treatment and prophylactic or preventative measures, wherein the object is to prevent or slow down (lessen) a medical condition, which includes but is not limited to diseases, symptoms and disorders.

A medical condition also includes a body's response to a disease or disorder, e.g., inflammation. Those in need of such treatment include those already with a medical condition as well as those prone to getting the medical condition or those in whom a medical condition is to be prevented.

As used herein, the term “therapeutically effective amount” of a compound is intended to refer to an amount that is sufficient or capable of preventing or at least slowing down (lessening) a medical condition, such as infectious diseases, respiratory illnesses (e.g., coronavirus caused by the SARS-CoV-2 virus or flu caused by influenza virus), cancer. Dosages and administration of compounds, compositions and formulations of the present disclosure may be determined by one of ordinary skill in the art of clinical pharmacology or pharmacokinetics. An effective amount of the active agent of the present disclosure to be employed therapeutically will depend, for example, upon the therapeutic objectives, the route of administration, and the condition of the patient. Accordingly, it may be necessary for the therapist to titer the dosage and modify the route of administration as required to obtain the optimal therapeutic effect.

The term “subject” is intended to broadly refer to any animal, such as a mammal, and including humans. Exemplary subjects include but are not limited to humans and non-human primates. The term “subject” as used herein also includes patients and non-patients. The term “patient” refers to individuals suffering or are likely to suffer from a medical condition such as infectious diseases (e.g., coronavirus caused by the SARS-CoV-2 virus), while “non-patients” refer to individuals not suffering and are likely to not suffer from the medical condition. “Non-patients” include healthy individuals, non-diseased individuals and/or an individual free from the medical condition. As used herein, the term “mammal” includes vertebrate such as a human or a large veterinary mammal (e.g., horses, cattle, deer, sheep, llamas, goats, pigs).

The term “bond” refers to a linkage between atoms in a compound or molecule. The bond may be a single bond, a double bond, or a triple bond.

The term “alkyl” as a group or part of a group refers to a straight or branched aliphatic hydrocarbon group having 1 to 20 carbon atoms, 1 to 10 carbon atoms, 1 to 6 carbon atoms, or 1, 2, 3, 4, 5, 6, 7, 8, 9, 10, 11, 12, 13, 14, 15, 16, 17, 18, 19 or 20 carbon atoms. Examples of suitable straight and branched alkyl substituents include methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl and the like. The group may be a terminal group or a bridging group.

The term “alkenyl” as a group or part of a group denotes an aliphatic hydrocarbon group containing at least one carbon-carbon double bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of double bonds and the orientation about each double bond is independently E or Z.

Exemplary alkenyl groups include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. The group may be a terminal group or a bridging group.

The term “alkynyl” as a group or part of a group denotes an aliphatic hydrocarbon group containing at least one carbon-carbon triple bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of triple bonds. Exemplary alkynyl groups include, but are not limited to, acetylenyl, propynyl, 1-butynyl, 2-butynyl, 3-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl and the like. The group may be a terminal group or a bridging group.

The term “cyclic” as used herein broadly refers to a structure where one or more series of atoms are connected to form at least one ring. The term includes, but is not limited to, both saturated and unsaturated 5-membered and saturated and unsaturated 6-membered rings. Examples of groups having a cyclic structure include, but are not limited to, cyclopentane, cyclopentene, cyclohexane, cyclohexene, benzene and the like. The term “cyclic” as used herein includes “heterocyclic”.

The term “heterocyclic” as used herein broadly refers to a structure where two or more different kinds of atoms are connected to form at least one ring. For example, a heterocyclic ring may be formed by carbon atoms and at least another atom (i.e. heteroatom) selected from oxygen (O), nitrogen (N) or (NR) and sulfur (S), where R is independently a hydrogen or an organic group. The term also includes, but is not limited to, saturated and unsaturated 5-membered, and saturated and unsaturated 6-membered rings. Examples of groups having a heterocyclic structure include, but are not limited to furan, thiophene, 1 H-pyrrole, 2H-pyrrole, 1-pyrroline, 2-pyrroline, 3-pyrroline, 1-pyrazoline, 2-pyrazoline, 3-pyrazoline, 2-imidazoline, 3-imidazoline, 4-imidazoline, pyrazole, imidazole, oxazole, isoxazole, thiazole, isothiazole, 1,2,3-triazole, 1,2,4-triazole, 1,2,3-oxadiazole, disubstituted 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, tetrahydrofuran, tetrahydrothiophene, pyrrolidine, 1,3-dioxolane, 1,2-oxathiolane, 1,3-oxathiolane, pyrazolidine, imidazolidine, pyridine, pyridazine, pyrimidine, pyrazine, 1,2-oxazine, 1,3-oxazine, 1,4-oxazine, thiazine, 1,2,3-triazine, 1,2,4-triazine, 1,3,5-triazine, 2H-pyran, 4H-pyran, 2-pyrone, 4-pyrone, 1,4-dioxin, 2H-thiopyran, 4H-thiopyran, tetrahydropyran, thiane, piperidine, 1,4-dioxane, 1,2-dithiane, 1,3-dithiane, 1,4-dithiane, 1,3,5-trithiane, piperazine, morpholine, thiomorpholine and the like.

The term “amine group” or the like is intended to broadly refer to a group containing —NR₂, where R is independently a hydrogen or an organic group. The group may be a terminal group or a bridging group.

The term “amide group” or the like is intended to broadly refer to a group containing —C(═O)NR₂, where R is independently a hydrogen or an organic group. The group may be a terminal group or a bridging group.

The term “aryl” as a group or part of a group denotes (i) an optionally substituted monocyclic, or fused polycyclic, aromatic carbocycle (ring structure having ring atoms that are all carbon) preferably having from 5 to 20, or 5, 6, 7, 8, 9, 10, 11, 12, 13, 14, 15, 16, 17, 18, 19 or 20 carbon atoms per ring. Examples of aryl groups include but are not limited to phenyl, tolyl, xylyl, naphthyl, anthracenyl, phenanthrenyl, fluorenyl, indenyl or indanyl and the like.

The term “heteroaryl” as a group or part of a group refers to groups containing an aromatic ring (preferably a 5- or 6-membered aromatic ring) having one or more carbon atoms (for example 1 to 6 carbon atoms) in the ring replaced by a heteroatom. Suitable heteroatoms may include nitrogen (N) or (NH), oxygen (O) and sulfur (S). Examples of heteroaryl include but are not limited to thiophene, benzothiophene, benzofuran, benzimidazole, benzoxazole, benzothiazole, benzisothiazole, naphtha[2,3-b]thiophene, furan, isoindolizine, xantholene, phenoxatine, pyrrole, imidazole, pyrazole, pyridine, pyrazine, pyrimidine, pyridazine, tetrazole, indole, isoindole, 1 H-indazole, purine, quinoline, isoquinoline, phthalazine, naphthyridine, quinoxaline, cinnoline, carbazole, phenantridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl and the like.

The group may be a terminal group or a bridging group.

The term “halogen” represents chlorine, fluorine, bromine or iodine. The term “halide” represents chloride, fluoride, bromide or iodide.

The term “optionally substituted,” when used to describe a chemical structure or moiety, refers to the chemical structure or moiety wherein one or more of its hydrogen atoms is optionally substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship.

For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

It will also be appreciated that where priority is claimed to an earlier application, the full contents of the earlier application is also taken to form part of the present disclosure and may serve as support for embodiments disclosed herein.

### DESCRIPTION OF EMBODIMENTS

Exemplary, non-limiting embodiments of a compound for preparing lipid nanoparticles encapsulating an agent, a method of preparing said compound, a nanoparticle composition comprising said compound and related methods/uses thereto are disclosed hereinafter.

### Compound

There is provided a compound for preparing lipid nanoparticles. In various embodiments, the compound comprises one or more peptide units/blocks and/or derivative(s) thereof. For example, the compound may comprise one or more oligopeptides, polypeptides/poly(amino acids) and/or derivative(s) thereof. In various embodiments, the total number of peptide units/blocks and/or derivative(s) thereof (or total length of oligopeptides, polypeptides/poly(amino acids) and/or derivative(s) thereof) in the compound is adjustable as desired. Advantageously, in various embodiments, the compound is designed/configured to allow the hydrophilicity/hydrophobicity balance of said compound to be customizable by the adjustment of the number of peptide units/blocks and/or derivative(s) thereof (or length of the oligopeptides, polypeptides/poly(amino acids) and/or derivative(s) thereof and length of lipid attached to oligopeptides, polypeptides/poly(amino acids) and/or derivative(s) thereof). In various embodiments, the peptide unit (e.g., oligopeptide or polypeptide) and/or derivative(s) thereof is further functionalized with/conjugated with one or more sugar/carbohydrate/saccharide unit(s)/group(s) and/or derivative(s) thereof. Advantageously, the presence of sugar/carbohydrate/saccharide unit(s)/group(s) and/or derivative(s) thereof equips the compound with or imparts the ability of targeting sugar/carbohydrate/saccharide receptors found in/on cell surfaces (e.g., surfaces of immune cells such as macrophages and dendritic cells). In various embodiments, the sugar/carbohydrate/saccharide and/or derivatives thereof is/are hydrophilic. Advantageously, the presence of sugar/carbohydrate/saccharide and/or derivatives thereof increases the hydrophilicity of the compound, and consequently solubility of the compound. Advantageously, the structure of the compound allows for embodiments of the compound to be used/formulated into nanoparticles in a composition that may be used as an encapsulation/loading agent, delivery vehicle/system and/or transfection vehicle/system. In various embodiments, the design of the compound helps prevent non-specific protein absorption, particle aggregation and controls the size of the nanoparticles formed. In various embodiments, embodiments of the compound help maintain colloidal stability (of the nanoparticles), and facilitate the condensation and encapsulating/loading of molecules/cargoes into the nanoparticle composition. In various embodiments, the compound is designed/configured to allow loading/encapsulation of one or more types of molecules or cargoes. In various embodiments, the compound is also designed/configured to allow the loaded/encapsulated agent to be released from a composition containing said compound and/or subsequently delivered to a desired target (e.g., cell, cytosol, tissue or organ). The molecules/cargoes to be loaded/encapsulated onto/into/within a composition containing the compound may include but is not limited to a therapeutic agent, a prophylactic agent, a biological agent or the like. In various embodiments, the molecules/cargoes to be loaded/encapsulated comprises a nucleic acid. For example, the molecules/cargoes to be loaded/encapsulated may be a nucleic acid selected from ribonucleic acid (RNA), messenger ribonucleic acid (mRNA), small interfering ribonucleic acid (siRNA), deoxyribonucleic acid (DNA), plasmid deoxyribonucleic acid (pDNA), oligonucleotides such as antisense oligonucleotide (ASO) or the like or combinations thereof. In various embodiments, the molecules/cargoes to be loaded/encapsulated comprises therapeutics. For example, the molecules/cargoes to be loaded/encapsulated may be therapeutics selected from drug molecule, vaccine or the like or combinations thereof. Advantageously, the compound is suitable for use in formulating into nanoparticles for encapsulating and/or delivering one or more therapeutic agent, prophylactic agent and/or biological agent to a desired target (e.g., subject, cell, cytosol, tissue or organ).

Accordingly, in various embodiments, there is also provided a carrier, nanocarrier or delivery system/vehicle comprising the compound.

In various embodiments, the compound comprises a structure that is represented by general formula (1):

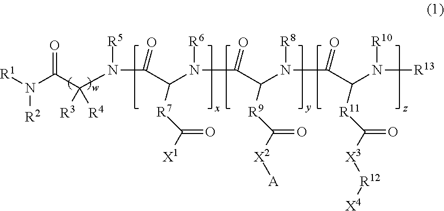

wherein
A comprises a hydrophilic moiety or component selected from carbohydrate/sugar/saccharide and derivatives thereof;
X¹ is —OR^{a} or —NR^{b}R^{c};
X⁴ is —OR^{y}, —SR^{z} or a hydrophilic group/tail/chain, where R^{y} to R^{z} are independently selected from the group consisting of H, optionally substituted alkyl, optionally substituted alkenyl or optionally substituted alkynyl;
X² and X³ are each independently —O— or —NR^{d}—, where R^{a} to R^{d} are independently selected from the group consisting of H, optionally substituted alkyl, optionally substituted alkenyl or optionally substituted alkynyl; R¹ and R² are each independently a hydrophobic group/tail/chain;
R³, R⁴, R⁵, R⁶, R⁸ and R¹⁰ are each independently H, optionally substituted alkyl, optionally substituted alkenyl or optionally substituted alkynyl;
is R⁷, R⁹, R¹¹ and R¹² are each independently optionally substituted alkyl, optionally substituted alkenyl or optionally substituted alkynyl;
R¹³ is H, optionally substituted alkyl, optionally substituted alkenyl or optionally substituted alkynyl, or —C(═O)R¹⁴, where R¹⁴ is optionally substituted alkyl, optionally substituted alkenyl, optionally substituted alkynyl;
w≥1;
x is 0 or 1;
y is 0 or ≥1; and
z is 0 or ≥1.

In various embodiments, the compound comprises one or more peptide units/blocks and/or derivative(s) thereof selected from general formula (X), (Y) and (Z):

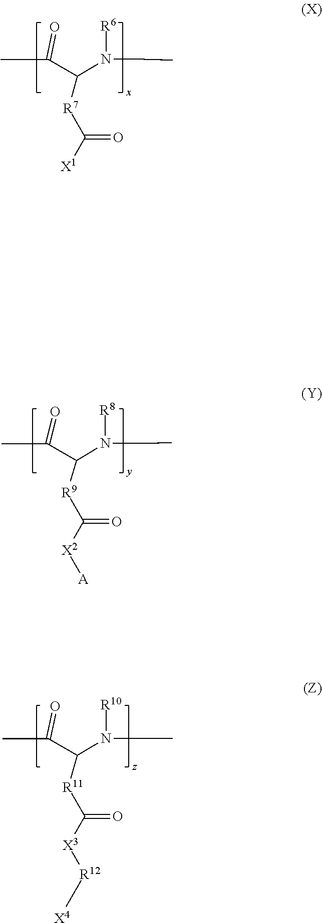

It should be appreciated that the arrangement of X, Y and Z may not follow the order shown in the structure of general formula (1) above and they may be randomly or regularly arranged in any other manner. It should also be appreciated that not all X, Y and Z may be present in the compound. For example, Z may be absent and the compound may comprise only X and Y.

In various embodiments, the peptide units/blocks and/or derivative(s) thereof may be joined in a regular/orderly/sequentially manner, or may be randomly distributed in/within the structure of the compound. For example, the compound may comprise a block polymer comprising repeating units arranged in X sequence, Y sequence, Z sequence, XY sequence, YX sequence, XZ sequence, ZX sequence, YZ sequence, ZY sequence, XYZ sequence, XZY sequence, YXZ sequence, YZX sequence, ZXY sequence or ZYX sequence, where X, Y and Z represent different types of repeating units represented by general formula (X), (Y) and (Z) respectively. In various embodiments, the compound comprises a polypeptide/poly(amino acid) and/or derivative(s) thereof, e.g., a block polypeptide and/or derivative(s) thereof.

In various embodiments, the peptide units/blocks and/or derivative(s) thereof are functionalized with/conjugated to a hydrophilic moiety (i.e. A) selected from carbohydrate/sugar/saccharide and derivatives thereof. In various embodiments, the carbohydrate/sugar/saccharide is selected from the group consisting of monosaccharide, disaccharide, oligosaccharide, polysaccharide and derivatives thereof. In various embodiments, the carbohydrate/saccharide is in a cyclic form, for example as a 5-membered ring (e.g., fructose or ribose) or 6-membered ring (e.g., mannose, glucose, galactose), or in a linear form. In various embodiments, the compound comprises a carbohydrate-functionalized polypeptide or carbohydrate-functionalized poly(amino acid). In various embodiments, the compound comprises a monosaccharide-functionalized polypeptide such as a mannose-functionalized polypeptide where a mannose is attached onto some units of the peptide in the polypeptide. In various embodiments, the compound comprises a monosaccharide-functionalized polypeptide such as a mannose-functionalized polypeptide where a mannose is attached onto each unit of the peptide in the polypeptide.

Advantageously, the presence of carbohydrate/sugar/saccharide and/or derivatives thereof allows embodiments of the compound to be capable of targeting sugar/carbohydrate/saccharide receptors found in/on cell surfaces (e.g., immune cell surfaces). In various embodiments, the design of the compound allows easy access of the carbohydrate to the cell (e.g., immune cell) in order to target carbohydrate receptors on the cell surface (e.g., immune cell surface), thereby prolonging the plasma half-life of nucleic acid and enhancing vaccination efficiency. Advantageously, the presence of sugar/carbohydrate/saccharide and/or derivatives thereof increases the hydrophilicity of the compound, and consequently solubility of the compound. Advantageously, in various embodiments, the presence of carbohydrate/sugar/saccharide and/or derivatives thereof in the compound eliminates the requirement of a hydrophilic polyethylene glycol (PEG) which is otherwise necessary in a conventional PEG-lipid conjugate. In various embodiments, the compound is substantially devoid of polyethylene glycol (PEG). Advantageously, in various embodiments, the design of the structure of the compound allows said compound to be used, in lieu of or as a substitute/replacement for a conventional PEG-lipid conjugate (e.g., ALC-0159).

It will be appreciated that various sugar molecules may be used as A in embodiments of the compound represented as general formula (1) as long as the sugar molecule is capable of providing a targeting ability and/or imparting hydrophilicity as a form of replacement of PEG.

In various embodiments, A is represented by general formula (2) having a 6-membered ring structure:

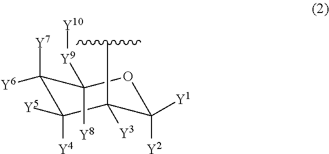

wherein
Y¹ to Y⁸ and Y¹⁰ are each independently selected from —H or —OH; and Y⁹ is alkyl.

In various embodiments, Y⁹ is optionally substituted —CₘH₂ₙ—, where m is from about 1 to about 20. For example, Y⁹ may be —CH₂—, —C₂H₄—, —C₃H₆—, —C₄H₈—, —C₅H₁₀—, —C₆H₁₂—, —C₇H₁₄—, —C₈H₁₆—, —C₉H₁₃—, or —C₁₀H₂₀—.

In various embodiments, A is represented by general formula (2A) and/or (2B) having a 6-membered ring structure:

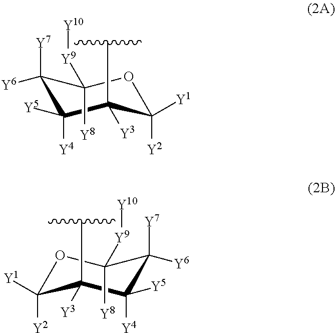

wherein Y¹ to Y¹⁰ contain one or more features and/or share one or more properties that are similar to those described above (e.g., as defined in general formula (2)). In various embodiments, A comprises D-sugar and/or L-sugar. For example, A may comprise D-mannose and/or L-mannose.

In various embodiments, the peptide units/blocks and/or derivative(s) thereof are functionalized with/conjugated to an alkanolamine and/or derivatives thereof (i.e. —X³—R¹²—X⁴). In various embodiments, the alkanolamine is selected from the group consisting of methanolamine, ethanolamine, 2-amino-2-methyl-1-propanol, valinol, or the like or combinations thereof. Advantageously, the presence of an alcohol and/or derivatives thereof, resulting from the reaction between the alkanolamine and a carboxylic acid, allows embodiments of the compound to shield negatively charged groups (e.g., —C(═O)O—) present in the compound for some applications (e.g., delivering RNA to the lungs, the heart or other organs). Advantageously, in various embodiments, the presence of an alkanolamine and/or derivatives thereof allows neutralization of negatively charged subunits within the polypeptide, which reduces possible repulsion between charged subunits. Even more advantageously, such neutralization effect (imparted by the alkanolamine and/or derivatives thereof) allows embodiments of the compound to be formulated into particles with customizable/adjustable particle size and zeta potential. For example, small particles with small size distribution (i.e. narrow PDI) and/or with the desired zeta potential (or surface charge) may be formulated from the compound. Advantageously, in various embodiments, reducing repulsion between charged subunits can reduce competition between charged groups in the polypeptides and nucleic acid (e.g., mRNA) for binding the ionizable lipid, and thus allowing for better encapsulation of the nucleic acid (e.g., mRNA).

In various embodiments, X¹ is —OR^{a} or —NR^{b}R^{c}, In various embodiments, X⁴ is —OR^{y}. In various embodiments, X² and X³ are each independently —O— or —NR^{d}—. In various embodiments, R^{a} to R^{d} and R^{y} are independently selected from the group consisting of H, optionally substituted alkyl, optionally substituted alkenyl or optionally substituted alkynyl.

In various embodiments, the compound is represented by general formula (1A):

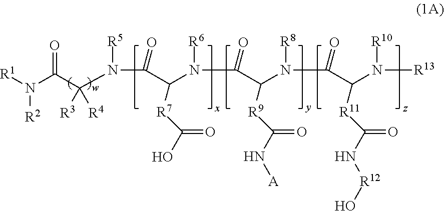

In various embodiments, the compound comprises a lipid compound. Accordingly, in various embodiments therefore, the term “compound” may comprise and/or may be used interchangeably with the terms “lipid polypeptide”, “lipid-polypeptide”, “polypeptide lipid”, “polypeptide-lipid”, “lipid-block-polypeptide”, “carbohydrate-functionalized lipid-polypeptide”, “sugar-functionalized lipid-polypeptide”, “mannose-functionalized lipid-polypeptide”, or the like.

In various embodiments, the compound comprises hydrophobic parts/tails/chains/groups at both R¹ and R². In various embodiments, R¹ and R² each independently contains at least linear aliphatic, branched aliphatic and/or cyclic hydrocarbons. In various embodiments, the hydrophobic tail/chain/group at R¹ and R² each independently comprises optionally substituted alkyl. In various embodiments, the hydrophobic tail/chain/group at R¹ and R² each independently comprises unsaturated hydrocarbons such as an optionally substituted alkenyl. For example, R¹ and R² may contain one or more CC double bond(s). The alkyl or alkenyl may have at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 carbon atoms. For example, R¹ and R² may be each independently CₚH₂ₚ₊₁ or CₚH₂ₚ, where p≥5, p≥6, p≥7, p≥8, p≥9, p≥10, p≥11, p≥12, p≥13, p≥14, p≥15, p≥16, p≥17, p≥18, p≥19, p≥20, p≥21, p≥22, p≥23, p≥24, p≥25, p≥26, p≥27, p≥28, p≥29, or p≥30. Advantageously, in various embodiments, the presence of hydrophobic parts/tails/chains/groups in the compound allows for integration of the compound into the lipid domain of lipid nanoparticles (LNPs), presenting the sugar/carbohydrate/saccharide-functionalized polypeptide on the surface of the LNPs for stability and cell-targeting ability.

In various embodiments, R³, R⁴, R⁵, R⁶, R⁸ and R¹⁰ are each independently H, optionally substituted alkyl, optionally substituted alkenyl or optionally substituted alkynyl. In various embodiments, R⁷, R⁹, R¹¹ and R¹² are each independently optionally substituted alkyl, optionally substituted alkenyl or optionally substituted alkynyl. For example, R³, R⁴, R⁵, R⁶, R⁷, R⁸, R⁹, R¹⁰, R¹¹ and R¹² may be selected from methyl, ethyl, n-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, or the like or combinations thereof.

In various embodiments, R⁷, R⁹, R¹¹ and R¹² are each independently optionally substituted —C_{q}H_{2q}—, where q is from about 1 to about 20. For example, R⁷, R⁹, R¹¹ and R¹² may be —CH₂—, —C₂H₄—, —C₃H₆—, —C₄H₈—, —C₅H₁₀—, —C₆H₁₂—, —C₇H₁₄—, —C₈H₁₆—, —C₉H₁₈—, or —C₁₀H₂₀—.

In various embodiments, R³ and R⁴ are H. In various embodiments, R⁵, R⁶, R⁸ and R¹⁰ are H.

In various embodiments, R¹³ is H, optionally substituted alkyl, optionally substituted alkenyl or optionally substituted alkynyl. For example, R¹³ may be selected from methyl, ethyl, n-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, or the like or combinations thereof.

In various embodiments, R¹³ is —C(═O)R¹⁴, where R¹⁴ is optionally substituted alkyl, optionally substituted alkenyl, optionally substituted alkynyl. For example, the primary amine group of the compound may be capped with —C(═O)R¹⁴ where R¹⁴ may be selected from methyl, ethyl, n-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, or the like or combinations thereof. In such embodiments, the compound is represented by general formula (11B):

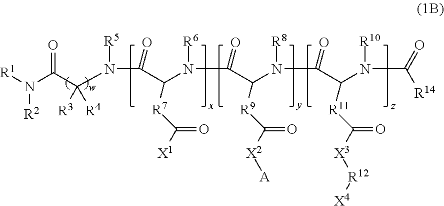

In various embodiments, w is an integer ≥1. In various embodiments, w≥1, w≥2, w≥3, w≥4, w≥5, w≥6, w≥7, w≥8, w≥9, or w≥10.

Advantageously, in various embodiments, the compound is designed/configured to allow the hydrophilicity/hydrophobicity balance of said is compound to be customizable by adjusting the length or hydrophobicity of R¹ and R², adjusting the number of peptide units/blocks and/or derivative(s) thereof (or length of the oligopeptides or polypeptides/poly(amino acids) and/or derivative(s) thereof), that is, by adjusting/altering/tuning the value of x, y, z, and/or controlling the degree of polymerization (i.e. value of n).

In various embodiments, x is 0 or an integer 1. In various embodiments, x=0. In various embodiments, x≥1, x≥2, x≥3, x≥4, x≥5, x≥6, x≥7, x≥8, x≥9, x≥10, x≥11, x≥12, x≥13, x≥14, x≥15, x≥16, x≥17, x≥18, x≥19, x≥20, x≥21, x≥22, x≥23, x≥24, x≥25, x≥26, x≥27, x≥28, x≥29, x≥30, x≥31, x≥32, x≥33, x≥34, x≥35, x≥36, x≥37, x≥38, x≥39, x≥40, x≥41, x≥42, x≥43, x≥44, x≥45, x≥46, x≥47, x≥48, x≥49, x≥50, x≥51, x≥52, x≥53, x≥54, x≥55, x≥56, x≥57, x≥58, x≥59, x≥60, x≥61, x≥62, x≥63, x≥64, x≥65, x≥66, x≥67, x≥68, x≥69, x≥70, x≥71, x≥72, x≥73, x≥74, x≥75, x≥76, x≥77, x≥78, x≥79, x≥80, x≥81, x≥82, x≥83, x≥84, x≥85, x≥86, x≥87, x≥88, x≥89, x≥90, x≥91, x≥92, x≥93, x≥94, x≥95, x≥96, x≥97, x≥98, x≥99, or x≥100.

In various embodiments, y is 0 or an integer ≥1. In various embodiments, y≥0. In various embodiments, y≥1, y≥2, y≥3, y≥4, y≥5, y≥6, y≥7, y≥8, y≥9, y≥10, y>11, y≥12, y≥13, y≥14, y≥15, y≥16, y≥17, y≥18, y≥19, y≥20, y≥21, y≥22, y≥23, y≥24, y≥25, y≥26, y≥27, y≥28, y≥29, y≥30, y≥31, y≥32, y≥33, y≥34, y≥35, y≥36, y≥37, y≥38, y≥39, y≥40, y≥41, y≥42, y≥43, y≥44, y≥45, y≥46, y≥47, y≥48, y≥49, y≥50, y≥51, y≥52, y≥53, y≥54, y≥55, y≥56, y≥57, y≥58, y≥59, y≥60, y≥61, y≥62, y≥63, y≥64, y≥65, y≥66, y≥67, y≥68, y≥69, y≥70, y≥71, y≥72, y≥73, y≥74, y≥75, y≥76, y≥77, y≥78, y≥79, y≥80, y≥81, y≥82, y≥83, y≥84, y≥285, y≥86, y≥87, y≥88, y≥89, y≥90, y≥91, y≥92, y≥93, y≥94, y≥95, y≥96, y≥97, y≥98, y≥299, or y≥100.

In various embodiments, z is 0 or an integer ≥1. In various embodiments, z≥0. In various embodiments, z≥1, z≥2, z≥3, z≥4, z≥5, z≥6, z≥7, z≥8, z≥9, z≥10, z≥11, z≥12, z≥13, z≥14, z≥15, z≥16, z>17, z>18, z≥19, z≥20, z>21, z≥22, z≥23, z≥24, z≥25, z≥26, z≥27, z≥28, z≥29, z≥30, z≥31, z≥32, z≥33, z≥34, z≥35, z≥36, z≥37, z≥38, z≥39, z≥40, z≥41, z≥42, z≥43, z≥44, z≥45, z≥46, z≥47, z≥48, z≥49, z≥50, z≥51, z≥52, z≥53, z≥54, z≥55, z≥56, z≥57, z≥58, z≥59, z≥60, z≥61, z≥262, z≥63, z≥64, z≥65, z≥66, z≥67, z≥68, z≥69, z≥70, z≥71, z≥72, z≥73, z≥74, z≥75, z≥76, z≥77, z≥78, z≥79, z≥80, z≥81, z≥82, z≥83, z≥84, z≥85, z≥86, z≥87, z≥88, z≥89, z≥90, z≥91, z≥92, z≥93, z≥94, z≥95, z≥96, z≥97, z≥98, z≥99, or z≥100.

In various embodiments, the total sum of x+y+z=n. In various embodiments, n is an integer 1. In various embodiments, n≥1, n≥2, n≥3, n≥4, n≥5, n≥6, n≥7 n≥8, n≥9, n≥10, n≥11, n≥12, n≥13, n≥14, n≥15, n≥16, n≥17, n≥18, n≥19, n≥20, n≥21, n≥22, n≥23, n≥24, n≥25, n≥26, n≥27, n≥28, n≥29, n≥30, n≥31, n≥32, n≥33, n≥34, n≥35, n≥36, n≥37, n≥38, n≥39, n≥40, n≥241, n≥42, n≥43, n≥44, n≥45, n≥246, n≥47, n≥48, n≥49, n≥50, n≥51, n≥52, n≥53, n≥54, n≥55, n≥56, n≥57, n≥58, n≥59, n≥60, n≥61, n≥62, n≥63, n≥64, n≥65, n≥66, n≥67, n≥68, n≥69, n≥70, n≥71, n≥72, n≥73, n≥74, n≥75, n≥76, n≥77, n≥78, n≥79, n≥80, n≥81, n≥82, n≥83, n≥84, n≥85, n≥86, n≥87, n≥88, n≥89, n≥90, n≥91, n≥92, n≥93, n≥94, n≥95, n≥96, n≥97, n≥98, n≥99, n≥100, n≥101, n≥102, n≥103, n≥104, n≥105, n≥106, n≥107, n≥108, n≥2109, n≥110, n≥111, n≥112, n≥113, n≥114, n≥115, n≥116, n≥117, n≥118, n≥119, n≥120, n≥121, n≥122, n≥123, n≥124, n≥125, n≥126, n≥127, n≥128, n≥129, n≥130, n≥131, n≥132, n≥133, n≥134, n≥135, n≥136, n≥137, n≥138, n≥139, n≥140, n≥141, n≥142, n≥143, n≥144, n≥145, n≥146, n≥147, n≥148, n≥149, n≥150, n≥151, n≥152, n≥153, n≥154, n≥155, n≥156, n≥157, n≥158, n≥159, n≥160, n≥161, n≥162, n≥163, n≥164, n≥165, n≥166, n≥167, n≥168, n≥169, n≥170, n≥171, n≥172, n≥173, n≥174, n≥175, n≥176, n≥177, n≥178, n≥179, n≥180, n≥181, n≥182, n≥183, n≥184, n≥185, n≥186, n≥187, n≥188, n≥189, n≥190, n≥191, n≥192, n≥193, n≥194, n≥195, n≥196, n≥197, n≥198, n≥199, n≥200, n≥250, or n≥300.

In various embodiments, the compound has a functionalization degree of at least about 5.0%, at least about 10.0%, at least about 15.0%, at least about 20.0%, at least about 25.0%, at least about 30.0%, at least about 35.0%, at least about 40.0%, at least about 45.0%, at least about 50.0%, at least about 55.0%, at least about 60.0%, at least about 65.0%, at least about 70.0%, at least about 75.0%, at least about 80.0%, at least about 85.0%, at least about 90.0%, at least about 95.0%, at least about 96.0%, at least about 97.0%, at least about 98.0%, at least about 99.0%, at least about 99.5%, at least about 99.9%, or about 100%. In various embodiments, the compound has a functionalization degree of from about 5% to about 100%, from about 10% to about 95%, from about 15% to about 90%, from about 20% to about 85%, from about 25% to about 80%, from about 30% to about 75%, from about 35% to about 70%, from about 40% to about 65%, from about 45% to about 60%, from about 50% to about 55%.

In various embodiments, the compound has a number average molecular weight (Mₙ) of from about 1,000 g/mol to about 100,000 g/mol, from about 1,500 g/mol to about 90,000 g/mol, from about 2,000 g/mol to about 80,000 g/mol, from about 2,500 g/mol to about 70,000 g/mol, from about 3,000 g/mol to about 60,000 g/mol, from about 3,500 g/mol to about 55,000 g/mol, from about 4,000 g/mol to about 50,000 g/mol, from about 4,500 g/mol to about 45,000 g/mol, from about 5,000 g/mol to about 40,000 g/mol, from about 5,500 g/mol to about 35,000 g/mol, from about 6,000 g/mol to about 30,000 g/mol, from about 6,500 g/mol to about 25,000 g/mol, from about 7,000 g/mol to about 24,000 g/mol, from about 7,500 g/mol to about 23,000 g/mol, from about 8,000 g/mol to about 22,000 g/mol, from about 8,500 g/mol to about 21,000 g/mol, from about 9,000 g/mol to about 20,000 g/mol, from about 9,500 g/mol to about 19,000 g/mol, from about 10,000 g/mol to about 18,000 g/mol, from about 11,000 g/mol to about 17,000 g/mol, from about 12,000 g/mol to about 16,000 g/mol, from about 13,000 g/mol to about 15,000 g/mol, or about 14,000 g/mol.

In various embodiments, the compound comprises a structure selected from one or more of the following:

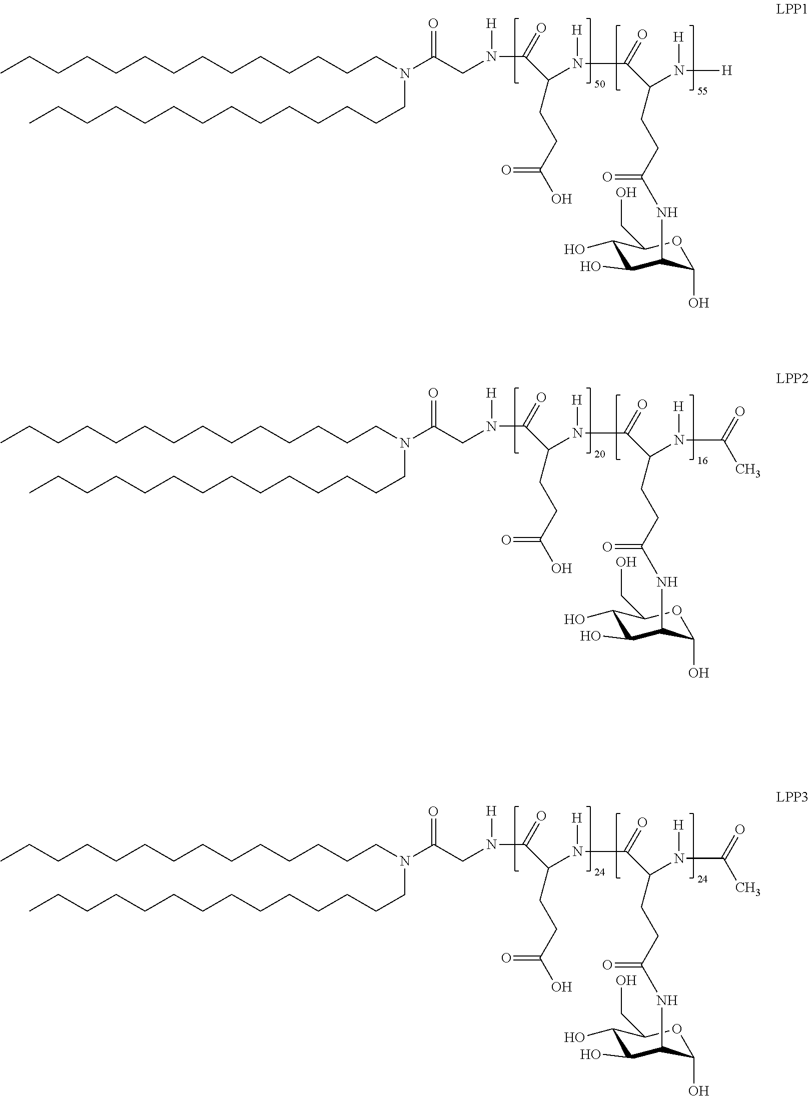

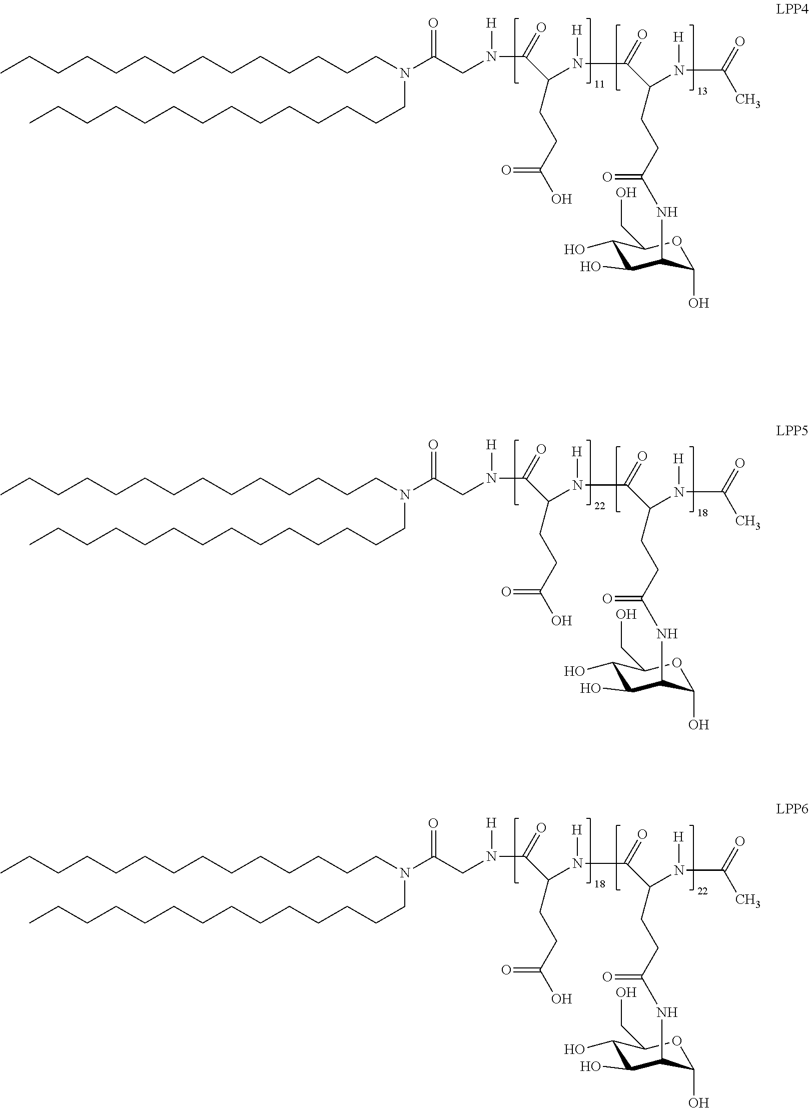

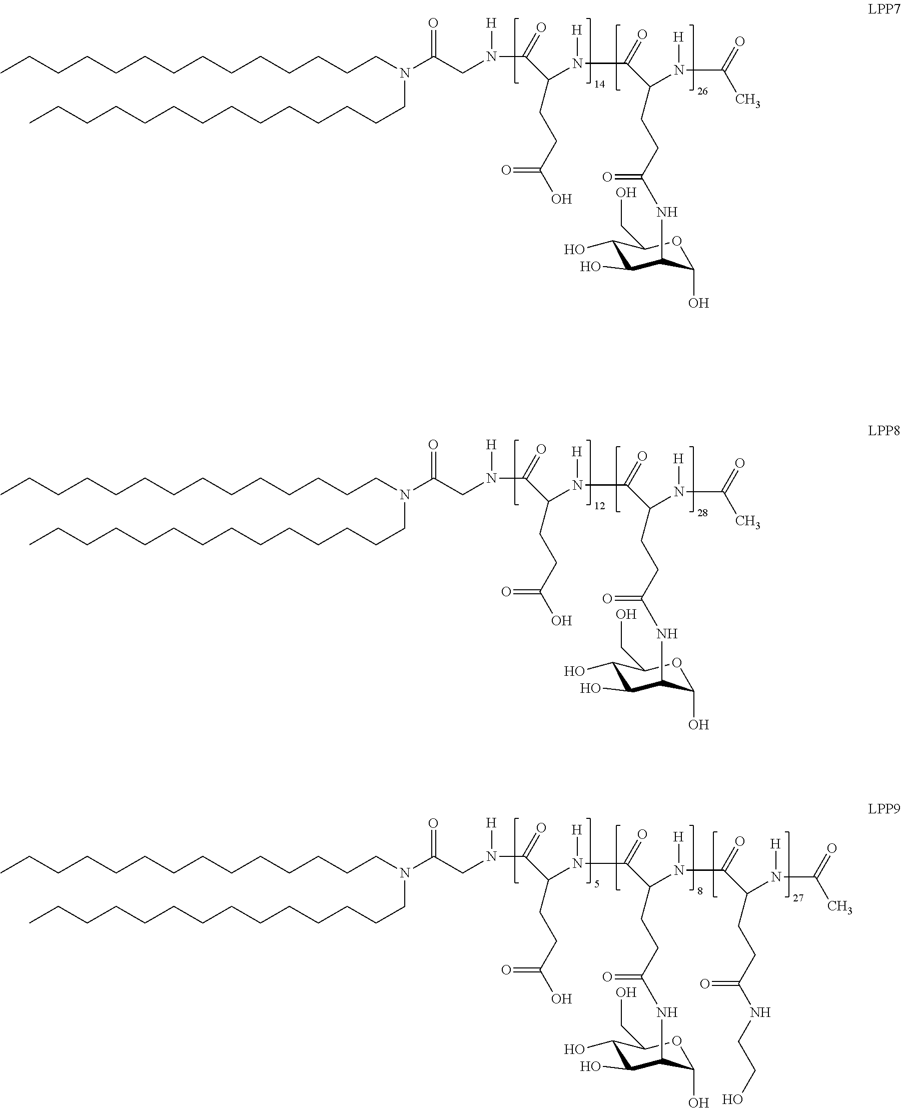

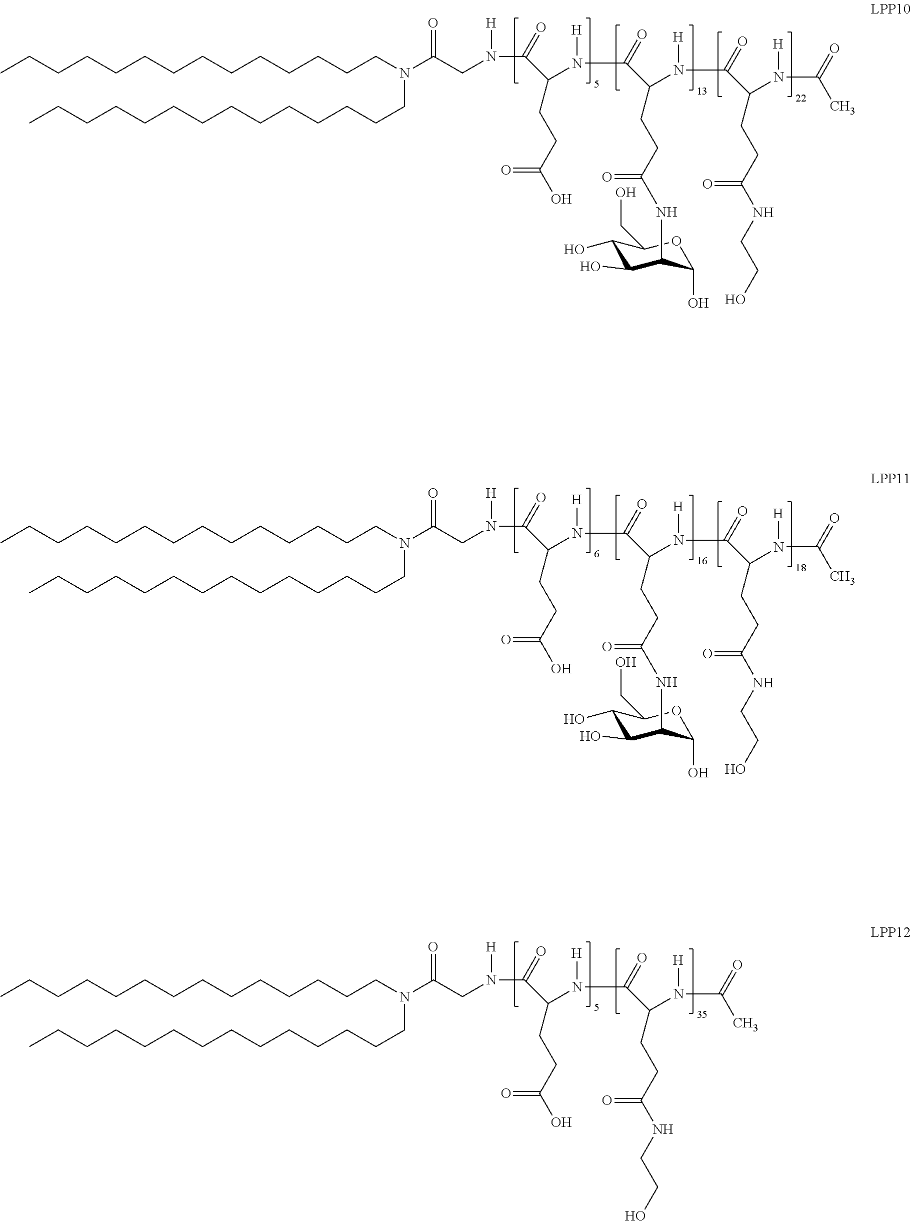

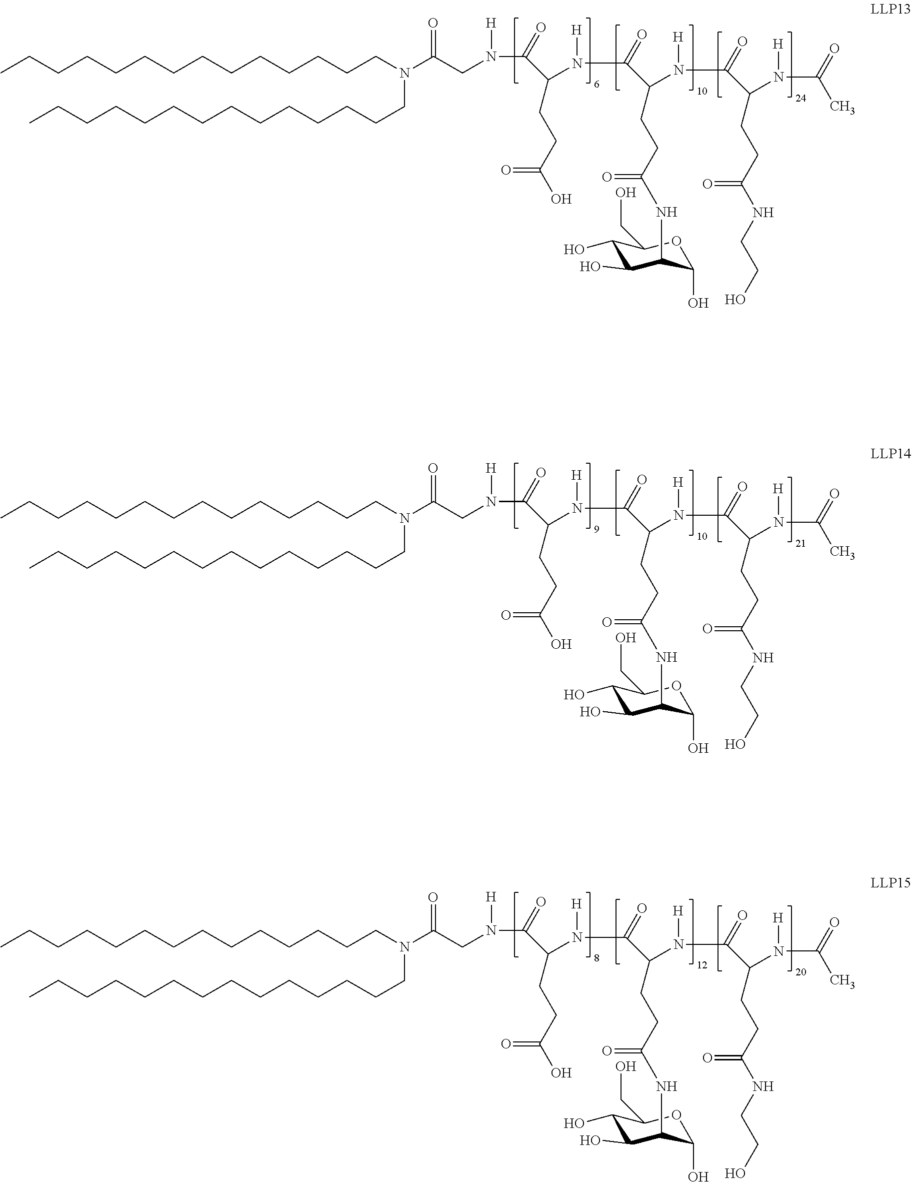

In various embodiments, D-mannose in LPP1 to LPP15 may be replaced with L-mannose.

### Method of Preparing Compound

There is provided a method of preparing a compound represented by general formula (1) as disclosed herein, the method comprising:
(a-i) polymerizing one or more N-carboxyanhydride (NCA) monomers represented by general formula (3) with a lipid initiator represented by general formula (4) to obtain a first intermediate compound represented by general formula (5):

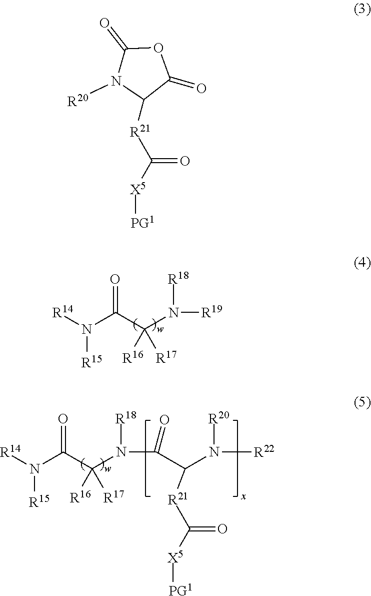

wherein
X⁵ is —O— or —NR^{e}—, where R^{e} is selected from the group consisting of H, optionally substituted alkyl, optionally substituted alkenyl or optionally substituted alkynyl;
R¹⁴ and R¹⁵ are each independently a hydrophobic group;
R¹⁶, R¹⁷, R¹⁸, R¹⁹, R²⁰ and R²² are each independently H, optionally substituted alkyl, optionally substituted alkenyl or optionally substituted alkynyl;
R²¹ is optionally substituted alkyl, optionally substituted alkenyl or optionally substituted alkynyl;
w≥1;
x is 0≥1; and
PG¹ is a protecting group selected from benzyl, tert-butyl or the like or combinations thereof;
(a-ii) optionally reacting the first intermediate compound represented by general formula (5) with an acylating agent represented by general formula (6) to obtain a second intermediate compound represented by general formula (7):

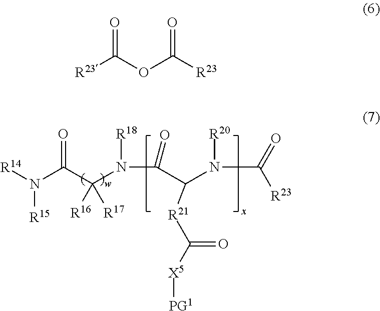

wherein
R²³ and R^{23′} are each independently optionally substituted alkyl, optionally substituted alkenyl or optionally substituted alkynyl;
(a-iii) deprotecting the first intermediate compound represented by general formula (5) and/or the second intermediate compound represented by general formula (7) to obtain a compound represented by general formula (8):

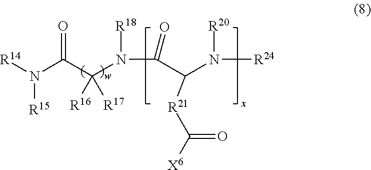

wherein
X⁶ is —OR^{a} or —NR^{f}R^{g}, where R^{f} to R^{g} are independently selected from the group consisting of H, optionally substituted alkyl, optionally substituted alkenyl or optionally substituted alkynyl; and
R²⁴ is H, optionally substituted alkyl, optionally substituted alkenyl or optionally substituted alkynyl, or —C(═O)R²⁵, where R²⁵ is optionally substituted alkyl, optionally substituted alkenyl, optionally substituted alkynyl;
(a-iv) reacting the compound represented by general formula (8) with at least one substrate selected from the following:
   (i) an amidating agent comprising a hydrophilic moiety represented by general formula (9); and
   (ii) an alkanolamine represented by general formula (10) to obtain a compound represented by general formula (11):

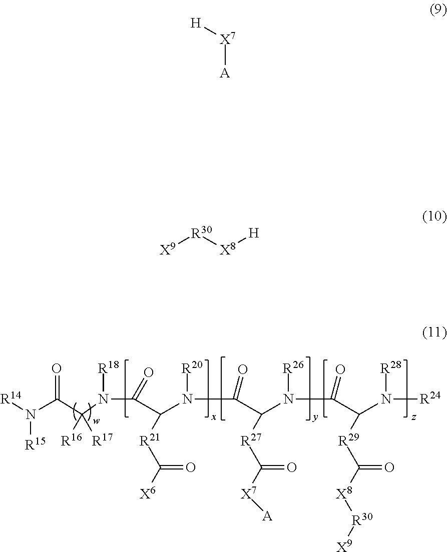

wherein
A comprises a hydrophilic moiety selected from carbohydrate/sugar/saccharide and derivatives thereof; R²⁶ and R²⁸ are each independently H, optionally substituted alkyl, optionally substituted alkenyl or optionally substituted alkynyl;
R²⁷, R²⁹ and R³⁰ are each independently optionally substituted alkyl, optionally substituted alkenyl or optionally substituted alkynyl;
X⁷ and X⁸ are each independently —O— or —NR^{h}—,
X⁹ is independently —ORⁱ or —NR^{j}R^{k}, where R^{h} to R^{k} are independently selected from the group consisting of H, optionally substituted alkyl, optionally substituted alkenyl or optionally substituted alkynyl;
y is 0 or 1; and
z is 0 or ≥1.

In various embodiments, the polymerizing step (a-i) comprises ring opening polymerization of the NCA ring/group. It will be appreciated that in step (a-i), the NCA monomers (i.e. represented by general formula (3)) may be added simultaneously to the initiator to polymerize and form random polypeptides or added sequentially to the initiator to form block polypeptides.

In various embodiments, the acylating agent represented by general formula (6) (used in step (a-ii)) comprises an acid anhydride. In various embodiments, R²³ and R^{23′} are each independently selected from methyl, ethyl, n-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, or the like or combinations thereof. In various embodiments, the acylating agent is selected from acetic anhydride (Ac₂O), acid halide (e.g., acyl halide), N-hydroxysuccinimide (NHS) esters, imidoesters or the like or combinations thereof.

In various embodiments, the deprotecting/deprotection step (a-iii) comprises subjecting the first intermediate compound represented by general formula (5) and/or the second intermediate compound represented by general formula (7) to acidic conditions. For example, the deprotection step (a-iii) may be carried out in the presence of one or more acids (e.g., trifluoroacetic acid (TFA), hydrobromic acid (HBr) and acetic acid (AcOH)).

In various embodiments, the reacting step (a-iv) is performed/carried in the presence of an activator and/or a base. In various embodiments, the activator is hexafluorophosphate benzotriazole tetramethyl uronium (HBTU) or the like. In various embodiments, the base is a non-nucleophilic base such as N,N-diisopropylethylamine (DIPEA) or the like.

In various embodiments, the reacting step (a-iv) comprises suspending/dispersing/mixing/dissolving the activator with the base in a molar ratio of from about 1:1 to about 1:5, about 1:1, about 1:1.5, about 1:2, about 1:2.5, about 1:3, about 1:3.5, about 1:4, about 1:4.5, or about 1:5.

In various embodiments, the reacting step (a-iv) comprises reacting the compound represented by general formula (8) with the amidating agent comprising a hydrophilic moiety represented by general formula (9) to obtain the compound represented by general formula (11), where x≥1, y≥1, z=0. In various embodiments, the reacting step (a-iv) comprises reacting the compound represented by general formula (8) with the alkanolamine represented by general formula (10) to obtain a compound represented by general formula (11), where x 1, y=0, z≥1. In various embodiments, the reacting step (a-iv) comprises reacting the compound represented by general formula (8) with the amidating agent comprising a hydrophilic moiety represented by general formula (9) and the alkanolamine represented by general formula (10) to obtain the compound represented by general formula (11), where x 1, y≥1, z≥1.

In various embodiments, the method further comprises, prior to step (a-i): (b-i) reacting a protected amino acid represented by general formula (12), where PG¹ is a protecting group, with a carbonylating agent to obtain the N-carboxyanhydride (NCA) monomer represented by general formula (3):

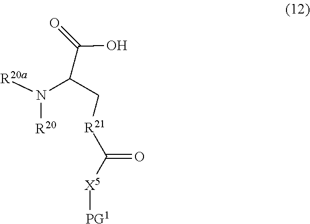

wherein R^{20a} is H, optionally substituted alkyl, optionally substituted alkenyl or optionally substituted alkynyl.

In various embodiments, the carbonylating agent (used in step (b-i)) is selected from phosgene, diphosgene, triphosgene, the like, or combinations thereof.

In various embodiments, the method further comprises, prior to step (a-i):
(c-i) reacting a lipid compound represented by general formula (13) with a protected amino acid represented by general formula (14) to obtain a protected lipid initiator represented by general formula (15):

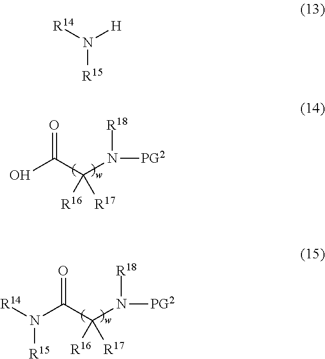

wherein PG² is a protecting group selected from tert-butoxycarbonyl (BOC), tert-butyl, benzyloxycarbonyl (Cbz), the like, or combinations thereof; and
(c-ii) deprotecting the protected lipid initiator represented by general formula is (15) to obtain the lipid initiator represented by general formula (4).

In various embodiments, the reacting step (c-i) is performed/carried in the presence of an activator and/or a base. In various embodiments, the activator is hexafluorophosphate benzotriazole tetramethyl uronium (HBTU) or the like. In various embodiments, the base is a non-nucleophilic base such as N,N-diisopropylethylamine (DIPEA) or the like.

In various embodiments, the reacting step (c-i) comprises suspending/dispersing/mixing/dissolving the activator with the base in a molar ratio of from about 1:1 to about 1:5, about 1:1, about 1:1.5, about 1:2, about 1:2.5, about 1:3, about 1:3.5, about 1:4, about 1:4.5, or about 1:5.

In various embodiments, the yield of the compound represented by general formula (11) (or compound represented by general formula (1)) is from about 1.0% to about 100.0%, from about 5.0% to about 99.0%, from about 10.0% to about 98.0%, from about 20.0% to about 97.0%, from about 30.0% to about 96.0%, from about 40.0% to about 95.0%, from about 50.0% to about 90.0%, from about 55.0% to about 85.0%, from about 60.0% to about 80.0%, from about 65.0% to about 70.0%, or about 75.0%.

Advantageously, embodiments of the method are straightforward to perform and/or have a low production/manufacturing cost (i.e. cost effective) as the reaction conditions are mild and do not require harsh and/or tedious step(s). Advantageously, embodiments of the method comprise simple purification steps (e.g., ease of isolation from by-products etc) and products are synthesized with high yields. Advantageously, embodiments of the method are scalable and/or have substantially high scalability.

In various embodiments, the polymerizing/reacting/deprotecting step (a-i), (a-ii), (a-iii), (a-iv), (b-i), (c-i) and/or (c-li) comprise(s) one or more of the following steps: dispersing, mixing, stirring, dissolving, sonicating and/or ultrasonicating.

In various embodiments, the polymerizing/reacting/deprotecting step (a-i), (a-ii), (a-iii), (a-iv), (b-i), (c-i) and/or (c-ii) is/are performed in the presence of an organic solvent. In various embodiments, any organic solvent that effectively serves as a medium to contain the components of the reaction mixture (e.g., reactants/substrates) may be used in embodiments of the reaction mixture disclosed herein. In various embodiments, the organic solvent is capable of substantially dissolving the components present in the reaction mixture. The organic solvent may be an organic solvent such as dichloromethane (DCM), tetrahydrofuran (THF), dimethysulfoxide (DMSO), acetonitrile, ethyl acetate, dimethylformamide (DMF) or the like or combinations thereof. In various embodiments, the organic solvent is provided in a dry or anhydrous form. For example, polymerizing step (a-i) may comprise suspending, dispersing, mixing, stirring, dissolving, sonicating and/or ultrasonicating the one or more N-carboxyanhydride (NCA) monomers represented by general formula (3) with an initiator represented by general formula (4) in the presence of dry/anhydrous organic solvent. For example, reacting step (b-i) may comprise suspending, dispersing, mixing, stirring, dissolving, sonicating and/or ultrasonicating the protected amino acid represented by general formula (12) with a carbonylating agent in the presence of dry/anhydrous organic solvent.

In various embodiments, the polymerizing/reacting/deprotecting step (a-i), (a-ii), (a-iii), (a-iv), (b-i), (c-i) and/or (c-ii) is/are carried out in an inert atmosphere. For example, the step(s) of dispersing, mixing and/or stirring may be performed in a glove box, the presence of an inert gas such as argon or nitrogen, or in the absence of reactive gases such as oxygen (e.g., dissolved oxygen).

In various embodiments, the polymerizing/reacting/deprotecting step (a-i), (a-ii), (a-iii), (a-iv), (b-i), (c-i) and/or (c-ii) is/are performed over a time duration of from about 1 hour to about 200 hours, from about 2 hours to about 150 hours, from about 3 hours to about 100 hours, from about 4 hours to about 90 hours, from about 5 hours to about 80 hours, from about 10 hours to about 70 hours, from about 20 hours to about 60 hours, from about 30 hours to about 50 hours, or about 40 hours. The polymerizing/reacting/deprotecting step (a-i), (a-ii), (a-iii), (a-iv), (b-i), (c-i) and/or (c-ii) may also be performed over a time duration of from about 1 hour to about 72 hours, from about 2 hours to about 60 hours, from about 3 hours to about 48 hours, from about 4 hours to about 36 hours, from about 5 hours to about 24 hours, or from about 6 hours to about 12 hours.

In various embodiments, the polymerizing/reacting/deprotecting step (a-i), (a-ii), (a-iii), (a-iv), (b-i), (c-i) and/or (c-ii) is/are performed at a temperature that is from about 10.0° C. to about 100.0° C., from about 20.0° C. to about 90.0° C., from about 30.0° C. to about 80.0° C., from about 40.0° C. to about 70.0° C., from about 50.0° C. to about 60.0° C., or about 55.0° C. In various embodiments, the polymerizing/reacting/deprotecting step (a-i), (a-ii), (a-iii), (a-iv), (b-i), (c-i) and/or (c-ii) is/are performed at room temperature e.g., that is from about 20° C. to about 30° C., about 21° C., about 22° C., about 23° C., about 24° C., about 25° C., about 26° C., about 27° C., about 28° C., about 29° C., or about 30° C.

In various embodiments, the deprotecting step (a-iii) and/or (c-li) is/are optionally performed at a temperature that is from about −10° C. to about 10° C., from about −9° C. to about 9° C., from about −8° C. to about 8° C., from about −7° C. to about 7° C., from about −6° C. to about 6° C., from about −5° C. to about 5° C., from about −4° C. to about 4° C., from about −3° C. to about 3° C., from about −2° C. to about 2° C., from about −1° C. to about 1° C., or 0° C., e.g., to control reaction kinetics. For example, the reacting step may be performed in an ice bath.

In various embodiments, the method further comprises:
(d-i) a step of isolating the first intermediate compound represented by general formula (5) after step (a-i);
(d-ii) a step of isolating the second intermediate compound represented by general formula (7) after step (a-ii);
(d-iii) a step of isolating the compound represented by general formula (8) after step (a-iii);
(d-iv) a step of isolating the compound represented by general formula (11) after step (a-iv);
(d-v) a step of isolating the N-carboxyanhydride (NCA) monomer represented by general formula (3) after step (b-i);
(d-vi) a step of isolating the protected lipid initiator represented by general formula (15) after step (c-i); and
(d-vii) a step of isolating the lipid initiator represented by general formula (4) after step (c-ii).

In various embodiments, the isolating step(s) comprises one or more of the following steps: re-dissolving, purifying, centrifuging, quenching, washing, precipitating and/or recrystallizing the first intermediate compound, the second intermediate compound, the compound represented by general formula (8), the compound represented by general formula (11), the N-carboxyanhydride (NCA) monomer represented by general formula (3), the protected lipid initiator represented by general formula (15) and/or the lipid initiator represented by general formula (4). The step(s) of purifying, centrifuging, quenching and/or washing may be repeated at least 1 time, at least 2 times, at least 3 times, at least 4 times, at least 5 times, at least 6 times, at least 7 times, at least 8 times, at least 9 times, at least 10 times, at least 15 times, at least 20 times with a washing medium. In various embodiments, the isolating step is performed to remove by-products from the first intermediate compound, the second intermediate compound, the compound represented by general formula (8), the compound represented by general formula (11), the N-carboxyanhydride (NCA) monomer represented by general formula (3), the protected lipid initiator represented by general formula (15) and/or the lipid initiator represented by general formula (4). In various embodiments, the washing medium comprises aqueous medium/solutions such as salt solution or deionized water. The salt solution may be bicarbonate salts such as sodium bicarbonate, chloride salts such as sodium chlorine (brine). In various embodiments, the salt solution comprises highly concentrated/saturated salt solution. In various embodiments, the washing medium may further comprise organic acid, e.g., weak organic acid such as citric acid.

In various embodiments, the method further comprises one or more of the following post reaction steps: drying the first intermediate compound, the second intermediate compound, the compound represented by general formula (8), the compound represented by general formula (11), the N-carboxyanhydride (NCA) monomer represented by general formula (3), the protected lipid initiator represented by general formula (15) and/or the lipid initiator represented by general formula (4), optionally under low temperature (e.g., freeze drying), under vacuum, and/or in an inert atmosphere.

### Nanoparticle Composition

Advantageously, in various embodiments, the design of the structure of the compound represented by general formula (1) allows said compound to be used, in lieu or in replacement/substitute of a conventional lipid-PEG conjugate (e.g., ALC-0159), in the formulation of nanoparticles in a composition. In various embodiments, embodiments of the compound are capable of being formulated into nanoparticles in a composition. Advantageously, in various embodiments, the design of the compound represented by general formula (1) helps prevent non-specific protein absorption, particle aggregation and controls the size of the nanoparticles formed. In various embodiments, embodiments of the compound represented by general formula (1) helps maintain colloidal stability (of the lipid nanoparticles), and facilitate the condensation and encapsulating/loading of molecules/cargoes into the nanoparticle composition.

The term “nanoparticles” may comprise and/or may be used interchangeably with the terms “lipid nanoparticles”, “encapsulated lipid nanoparticles”, “loaded lipid nanoparticles”, “LNPs” or the like.

There is provided a nanoparticle composition comprising:
(i) a compound represented by general formula (1) as disclosed herein; and
(ii) a therapeutic agent, prophylactic agent and/or biological agent that is encapsulated/loaded in said composition.

Advantageously, the composition is suitable for use in the encapsulation, delivery and/or transfection of one or more therapeutic agent, prophylactic agent and/or biological agent e.g., to a desired target (such as subject, cell, cytosol, tissue or organ).

In various embodiments, the composition further comprises:
(a) ionizable lipid;
(b) neutral/helper lipid; and
(c) sterol

In various embodiments, the composition is substantially devoid of polyethylene glycol (PEG). In various embodiments, the composition is substantially devoid of polyethylene glycol (PEG)-modified lipid conjugates, polyethylene glycol (PEG)-modified lipid, PEGylated lipid, PEG-conjugated lipid, PEG-lipid conjugate and/or lipid modified with PEG. In various embodiments, the compound is a substitute/replacement for PEG-lipid conjugate.

In various embodiments, the compound, ionizable lipid, neutral/helper lipid, and sterol are mixed/dissolved in an organic solvent. In various embodiments, the formation of lipid nanoparticle comprises self-assembly of the lipid components and one or more types of molecules or cargoes. In various embodiments, any organic solvent that effectively serves as a medium to contain the lipid components may be used in embodiments of the lipid materials disclosed herein. In various embodiments, the organic solvent is capable of substantially dissolving the components present in the mixture. The organic solvent may comprise ethanol, isopropanol, acetonitrile, ethyl acetate, methanol, tetrahydrofuran, dimethyl sulfoxide, dimethylformamide or the like or combinations thereof.

In various embodiments, the ionizable lipid, helper lipid, sterol, and compound represented by general formula (1) are mixed at a mole ratio of about 15-140:about 3-30:about 14-130:about 0.2-40, at a mole ratio of about 22.5-105:about 4.5-22.5:about 21-97.5:about 0.3-30, at a mole ratio of about 30-70:about 6-15:about 28-65:about 0.4-20, at a mole ratio of about 45-53:about 9-11:about 42-49:about 0.6-15, or at a mole ratio of about 48:about 10:about 45:about 17.

In various embodiments, the ionizable lipid is selected from ALC-0315, SM-102, Lipid 5, DLinDMA, D-Lin-MC2-DMA, DLin-MC3-DMA, D-Lin-MC4-DMA, Dlin-KC2-DMA, YSK05, AA3-Dlin, SSPaImM, SSPalmO-Phe, Lipid A9, L319, DODMA, CL1, BP Lipid 310, ATX-001, ATX-100, Lipid 2, 80-016B, BP Lipid 309, BP Lipid 307, 93-017S, 93-0170, NT1-014B, 306-012B-3, 306-012B, 113-016B, 306010, 3060i9-cis2, BAMEA-016B, Al-28, 113-012B, 98N12-5, Ckk-E12, OF-02, C12-200, BP Lipid 311, BP Lipid 308, BP Lipid 314, BP Lipid 312, LPO1, TCL053, Lipid C24, BP Lipid 315, Lipid 29, 9A1 P9, C13-112-tri-tail, C13-113-tri-tail, C13-112-tetra-tail, or C13-113-tetra-tail, C12-200 and combinations thereof. It will be appreciated that any suitable ionizable lipid that effectively modulates/adjusts/changes its charge depending on the environmental pH may be used in embodiments of the composition disclosed herein.

In various embodiments, the composition comprises from about 8.0 mol % to about 90.0 mol % of ionizable lipid. In various embodiments, the composition comprises from about 8.0 mol % to about 90.0 mol %, from about 9.0 mol % to about 85.0 mol %, from about 10.0 mol % to about 80.0 mol %, from about 11.0 mol % to about 75.0 mol %, from about 12.0 mol % to about 70.0 mol %, from about 13.0 mol % to about 65.0 mol %, from about 14.0 mol % to about 60.0 mol %, from about 15.0 mol % to about 55.0 mol %, from about 16.0 mol % to about 50.0 mol %, from about 17.0 mol % to about 45.0 mol %, from about 18.0 mol % to about 40.0 mol %, from about 19.0 mol % to about 35.0 mol %, from about 20.0 mol % to about 30.0 mol %, or about 25.0 mol % of ionizable lipid.

In various embodiments, the neutral/helper lipid comprises a phospholipid such as an unsaturated lipid. Examples of phospholipid includes, but are not limited to, 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and the like and combinations thereof.

In various embodiments, the composition comprises from about 1.0 mol % to about 55.0 mol % of neutral/helper lipid. In various embodiments, the composition comprises from about 1.0 mol % to about 55.0 mol %, from about 2.0 mol % to about 54.0 mol %, from about 3.0 mol % to about 53.0 mol %, from about 4.0 mol % to about 52.0 mol %, from about 5.0 mol % to about 51.0 mol %, from about 6.0 mol % to about 50.0 mol %, from about 7.0 mol % to about 49.0 mol %, from about 8.0 mol % to about 48.0 mol %, from about 9.0 mol % to about 47.0 mol %, from about 10.0 mol % to about 46.0 mol %, from about 15.0 mol % to about 45.0 mol %, from about 20.0 mol % to about 40.0 mol %, from about 25.0 mol % to about 35.0 mol %, or about 30.0 mol % of neutral/helper lipid.

In various embodiments, the sterol is selected from cholesterol, fecosterol, sitosterol, ergosterol, campesterol, stigmasterol, brassicasterol, avenasterol, or the like or combinations thereof.

In various embodiments, the composition comprises from about 7.0 mol % to about 90.0 mol % of sterol. In various embodiments, the composition comprises from about 7.0 mol % to about 90.0 mol %, from about 8.0 mol % to about 89.0 mol %, from about 9.0 mol % to about 88.0 mol %, from about 10.0 mol % to about 87.0 mol %, from about 11.0 mol % to about 86.0 mol %, from about 12.0 mol % to about 85.0 mol %, from about 13.0 mol % to about 84.0 mol %, from about 14.0 mol % to about 83.0 mol %, from about 15.0 mol % to about 82.0 mol %, from about 16.0 mol % to about 81.0 mol %, from about 17.0 mol % to about 80.0 mol %, from about 18.0 mol % to about 75.0 mol %, from about 19.0 mol % to about 70.0 mol %, from about 20.0 mol % to about 65.0 mol %, from about 25.0 mol % to about 60.0 mol %, from about 30.0 mol % to about 55.0 mol %, from about 35.0 mol % to about 50.0 mol %, or from about 40.0 mol % to about 45.0 mol % of sterol.

In various embodiments, the composition comprises from about 0.06 mol % to about 40.0 mol % of the compound represented by general formula (1). In various embodiments, the composition comprises from about 0.07 mol % to about 40.0 mol %, from about 0.08 mol % to about 39.0 mol %, from about 0.09 mol % to about 38.0 mol %, from about 0.10 mol % to about 37.0 mol %, from about 0.11 mol % to about 36.0 mol %, from about 0.12 mol % to about 35.0 mol %, from about 0.13 mol % to about 34.0 mol %, from about 0.14 mol % to about 33.0 mol %, from about 0.15 mol % to about 32.0 mol %, from about 0.20 mol % to about 31.0 mol %, from about 0.25 mol % to about 30.0 mol %, from about 0.50 mol % to about 25.0 mol %, from about 0.75 mol % to about 20.0 mol %, from about 1.0 mol % to about 15.0 mol %, from about 2.0 mol % to about 10.0 mol %, from about 3.0 mol % to about 9.0 mol %, from about 4.0 mol % to about 8.0 mol %, from about 5.0 mol % to about 7.0 mol %, or about 6.0 mol % of the compound represented by general formula (1).

In various embodiments, the therapeutic agent, prophylactic agent and/or biological agent is provided in an aqueous buffer. The aqueous buffer may be sodium acetate.

In various embodiments, the nanoparticle composition comprises nanoparticles formed from the compound represented by general formula (1).

### Nanoparticles

There is provided nanoparticles (e.g., lipid nanoparticles) comprising:
(i) the compound represented by general formula (1) as disclosed herein; and
(ii) a therapeutic and/or prophylactic agent and/or biological agent that is encapsulated/loaded/coupled/bonded/linked/bound in/to said nanoparticles.

In various embodiments, the nanoparticles have a N:P or N/P ratio (i.e. molar ratio of ionizable nitrogen atoms in the ionizable lipid to phosphate groups in the therapeutic agent, prophylactic agent and/or biological agent (e.g., nucleic acid) is from about 2:1 to about 40:1. The nanoparticles may have a N:P or N/P ratio that is from about 2:1 to about 40:1, from about 3:1 to about 39:1, from about 4:1 to about 38:1, from about 5:1 to about 37:1, from about 6:1 to about 36:1, from about 7:1 to about 35:1, from about 8:1 to about 34:1, from about 9:1 to about 33:1, from about 10:1 to about 32:1, from about 11:1 to about 31:1, from about 12:1 to about 30:1, from about 13:1 to about 29:1, from about 14:1 to about 28:1, from about 15:1 to about 27:1, from about 16:1 to about 26:1, from about 17:1 to about 25:1, from about 18:1 to about 24:1, from about 19:1 to about 23:1, from about 20:1 to about 22:1, or about 21:1.

In various embodiments, it will be appreciated that shorter nucleic acid therapeutics (e.g. siRNA) or prophylactic agents (e.g., mRNA) require more (i.e. a larger amount/concentration/volume of) ionizable lipids to encapsulate them into lipid nanoparticles. In various embodiments therefore, a N/P ratio of up to about 40:1 is used to encapsulate and deliver nucleic acid therapeutics (e.g. shorter nucleic acid therapeutics siRNA).

In various embodiments, the encapsulation/loading/binding efficiency of the therapeutic agent, prophylactic agent and/or biological agent in the composition/nanoparticles is at least about 5.0%, at least about 10.0%, at least about 15.0%, at least about 20.0%, at least about 25.0%, at least about 30.0%, at least about 35.0%, at least about 40.0%, at least about 45.0%, at least about 50.0%, at least about 55.0%, at least about 60.0%, at least about 65.0%, at least about 70.0%, at least about 75.0%, at least about 80.0%, at least about 85.0%, at least about 90.0%, at least about 95.0%, at least about 96.0%, at least about 97.0%, at least about 98.0%, at least about 99.0%, at least about 99.5%, at least about 99.9%, or about 100%.

In various embodiments, the nanoparticles have an encapsulation efficiency that is slightly lower, comparable to, no less or is higher than that of corresponding nanoparticles using ALC-0159 as the PEG-lipid conjugate under similar conditions. For example, the encapsulation efficiency may be at least about 50% of that of a corresponding nanoparticle using ALC-0159 as the PEG-lipid conjugate under similar conditions. In another example, the encapsulation efficiency may be at least about 1% to at least about 50% higher than that of corresponding nanoparticles using ALC-0159 as the PEG-lipid conjugate under similar conditions.

In various embodiments, the cell transfection efficiency (% of the cells that are transfected with the gene) of the composition/nanoparticles is at least about 1.0%, at least about 5.0%, at least about 10.0%, at least about 15.0%, at least about 20.0%, at least about 25.0%, at least about 30.0%, at least about 35.0%, at least about 40.0%, at least about 45.0%, at least about 50.0%, at least about 55.0%, at least about 60.0%, at least about 65.0%, at least about 70.0%, at least about 75.0%, at least about 80.0%, at least about 85.0%, at least about 90.0%, at least about 95.0%, at least about 96.0%, at least about 97.0%, at least about 98.0%, at least about 99.0%, at least about 99.5%, at least about 99.9%, or about 100%. In various embodiments, the cell transfection efficiency is not required to be 100%. For example, it will be appreciated that vaccine applications may not need/require to transfect 100% cells in order to mediate an immune response, unlike in the case of cancer therapy applications.

In various embodiments, the nanoparticles have a cell transfection efficiency that is comparable to, no less or is higher than that of corresponding nanoparticles using ALC-0159 as the PEG-lipid conjugate under similar conditions. For example, the cell transfection efficiency may be at least about 30% of that of a corresponding nanoparticle using ALC-0159 as the PEG-lipid conjugate under similar conditions. In another example, the cell transfection efficiency may be at least about 50% to at least about 1,000% higher than that of corresponding nanoparticles using ALC-0159 as the PEG-lipid conjugate under similar conditions.

In various embodiments, the transfection efficiency in certain cell lines is lower than that of corresponding nanoparticles using ALC-0159 as the PEG-lipid conjugate under similar conditions but still at the same order of magnitude. It will be appreciated that such level of gene transfection may still be applicable for gene therapy.

In various embodiments, the nanoparticles have an average or mean particle size (or diameter) of no more than about 800 nm, no more than about 750 nm, no more than about 700 nm, no more than about 650 nm, no more than about 600 nm, no more than about 550 nm, no more than about 500 nm, no more than about 450 nm, no more than about 400 nm, no more than about 350 nm, no more than about 300 nm, no more than about 250 nm, no more than about 200 nm, no more than about 150 nm, no more about 100 nm, no more than about 90 nm, no more than about 80 nm, no more than about 70 nm, no more than about 60 nm, no more than about 50 nm, no more than about 40 nm, no more than about 30 nm, no more than about 20 nm, or no more than about 10 nm. In various embodiments, the nanoparticles have an average or mean particle size (or diameter) of from about 10.0 nm to about 400.0 nm, from about 15.0 nm to about 300.0 nm, from about 20.0 nm to about 200.0 nm, from about 30.0 nm to about 190.0 nm, from about 40.0 nm to about 180.0 nm, from about 50.0 nm to about 170.0 nm, from about 60.0 nm to about 160.0 nm, from about 70.0 nm to about 150.0 nm, from about 80.0 nm to about 140.0 nm, from about 90.0 nm to about 130.0 nm, from about 100.0 nm to about 120.0 nm, or about 110.0 nm.

In various embodiments, the composition comprising the nanoparticles has a polydispersity index (PDI) of from about 0.01 to about 0.50, from about 0.0125 to about 0.45, from about 0.015 to about 0.40, from about 0.020 to about 0.35, from about 0.025 to about 0.30, from about 0.030 to about 0.25, from about 0.035 to about 0.20, from about 0.040 to about 0.15, from about 0.045 to about 0.10, from about 0.050 to about 0.095, from about 0.055 to about 0.090, from about 0.060 to about 0.085, from about 0.065 to about 0.080, or from about 0.070 to about 0.075. Advantageously, in various embodiments, the nanoparticles have a narrow particle size distribution (e.g., not more than about 0.2) and/or the nanoparticles or nanoparticle composition is relatively/substantially homogenous.

In various embodiments, the nanoparticles have a zeta potential of from about −50.0 mV to about +30.0 mV, from about −15.0 mV to about +15.0 mV, from about −14.0 mV to about +14.0 mV, from about −13.0 mV to about +13.0 mV, from about −12.0 mV to about +12.0 mV, from about −11.0 mV to about +11.0 mV, from about −10.0 mV to about +10.0 mV, from about −9.0 mV to about +9.0 mV, from about −8.0 mV to about +8.0 mV, from about −7.0 mV to about +7.0 mV, from about −6.0 mV to about +6.0 mV, from about −5.0 mV to about +5.0 mV, from about −4.0 mV to about +4.0 mV, from about −3.0 mV to about +3.0 mV, from about −2.0 mV to about +2.0 mV, from about −1.0 mV to about +1.0 mV, or about 0 mV in saline (e.g., phosphate-buffered saline (PBS)) or in a physiological environment. Advantageously, in various embodiments, the nanoparticles have a substantially neutral surface charge or a negative charge or a positive charge, making the nanoparticles suitable/desirable for in vivo applications. In various embodiments, the nanoparticles with a negative charge may be used to target spleen and lymph nodes. In various embodiments, the nanoparticles with a positive charge may be used for local delivery of cargos across the mucus layer, e.g. nasal delivery.

In various embodiments, the cell viability of the composition/nanoparticle is at least about 50.0%, at least about 60.0%, at least about 70.0%, at least about 80.0%, at least about 90.0%, at least about 95.0%, at least about 96.0%, at least about 97.0%, at least about 98.0%, at least about 99.0%, at least about 99.5%, or at least about 99.9%.

In various embodiments, the nanoparticle has a cell viability that is no less or is higher than that of a corresponding nanoparticle using ALC-0519 as the PEG-lipid conjugate under similar conditions. For example, the cell viability may be at least comparable to that of a corresponding nanoparticle using ALC-0519 as the PEG-lipid conjugate under similar conditions. Accordingly, in various embodiments, the nanoparticle comprises/possesses high cytocompatibility and/or negligible cytotoxicity.

In various embodiments, the composition/compound/nanoparticles is/are biocompatible, i.e. the composition/compound/nanoparticle is compatible with biological systems or parts of the biological systems without substantially or significantly eliciting an adverse physiological response such as a toxic reaction/response (e.g., cytotoxicity), an immune reaction/response, an injury or the like when used on the human or animal body. In various embodiments, the composition/compound/nanoparticle is substantially devoid of substances that elicit an adverse physiological response. It will be appreciated that the composition/compound/nanoparticle may trigger/elicit an immune response (e.g., to enhance vaccination efficacy), and in such embodiments, the composition/compound/nanoparticle is still considered to be biocompatible.

Advantageously, the nanoparticles (e.g., lipid nanoparticles) are capable of binding therapeutic agent, prophylactic agent and/or biological agent (e.g., RNA) effectively and/or providing high transfection efficiency without causing/inducing substantial or any cytotoxicity.

### Method of Preparing Nanoparticles

There is provided a method of preparing nanoparticles as disclosed herein, the method comprising:
(e-i) preparing an aqueous composition comprising therapeutic and/or prophylactic agent and/or biological agent;
(e-ii) mixing the aqueous composition obtained from (e-i) with the composition as disclosed herein to obtain nanoparticles.

In various embodiments, the step (e-i) comprises mixing therapeutic and/or prophylactic agent and/or biological agent in an aqueous buffer. The aqueous buffer may be sodium acetate, citrate buffer, phosphate buffer, glycine buffer solution, or the like or combinations thereof.

In various embodiments, the mixing step (e-i) is performed at a pH value of from about 2.5 to about 6.5, from about 2.6 to about 6.4, from about 2.7 to about 6.3, from about 2.8 to about 6.2, from about 2.9 to about 6.1, from about 3.0 to about 6.0, from about 3.1 to about 5.9, from about 3.2 to about 5.8, from about 3.3 to about 5.7, from about 3.4 to about 5.6, from about 3.5 to about 5.5, from about 3.6 to about 5.4, from about 3.7 to about 5.3, from about 3.8 to about 5.2, from about 3.9 to about 5.1, from about 4.0 to about 5.0, from about 4.1 to about 4.9, from about 4.2 to about 4.8, from about 4.3 to about 4.7, from about 4.4 to about 4.6, or about 4.5.

In various embodiments, the composition as disclosed herein comprises organic phase (e.g., ethanol). In various embodiments, the aqueous composition comprises aqueous phase. In various embodiments, the step (e-ii) comprises mixing the aqueous composition with the organic composition as described herein at a volume ratio of the aqueous phase to organic phase from about 10:1 to about 1:1. For example, the aqueous phase may be mixed with the organic phase at a volume ratio of from about 10:1 to about 1:1, at about 9:1, at about 8:1, at about 7:1, at about 6:1, at about 5:1, at about 4:1, at about 3:1, or at about 2:1.

In various embodiments, the step (e-ii) of mixing the aqueous composition with the organic composition comprises injecting (e.g., direct injecting) the organic composition into the aqueous composition.

In various embodiments, the step (e-ii) of mixing the aqueous composition with the composition comprises micro-mixing, e.g., microfluidic mixing using a microfluidic device. The micro-mixing may be performed via passive mixing using passive micromixers such as T-shaped or Y-shaped microfluidic mixers parallel lamination, sequential, focusing enhanced mixers or droplet micromixers. The micro-mixing may also be performed via active mixing using external forces such as pressure field, electrokinetic, dielectrophoretic, electrowetting, magneto-hydrodynamic or ultrasound. Advantageously, as microfluidic mixing comprises mixing the two compositions (i.e. aqueous composition and composition disclosed herein) in a controlled manner and/or with a specified/fixed/controlled/precise mixing ratio, the interaction between the two compositions (e.g., between ionizable lipid and therapeutic, prophylactic and/or biological agent) is regulated, thereby producing nanoparticles with a smaller particle size and/or with a narrow size distribution or homogeneity (e.g, smaller PDI).

In various embodiments, the method further comprises removing the organic phase (e.g. ethanol). For example, removing the organic phase may include dialysis or filtration. Advantageously, removal of the organic phase through dialysis or filtration may improve the encapsulation efficiency of the therapeutic and/or prophylactic agent and/or biological agent.

In various embodiments, there is also provided a carrier, nanocarrier or delivery system/vehicle comprising the composition/compound/nanoparticles as disclosed herein.

In various embodiments, there is also provided a vaccine composition comprising the composition/compound/nanoparticles as disclosed herein.

In various embodiments, there is also provided a carrier, a nanocarrier, a delivery system/vehicle, a compound, a nanoparticle composition, nanoparticles (or lipid nanoparticles) disclosed herein for use in medicine (e.g., for the treatment or prophylaxis of one or more of the diseases, disorders or conditions mentioned herein).

In various embodiments, there is also provided a carrier, a nanocarrier, a delivery system/vehicle, a compound, a nanoparticle composition, nanoparticles (or lipid nanoparticles) disclosed herein for use in the treatment or prophylaxis of a disease, disorder or condition, the use of said carrier, a nanocarrier, a delivery system/vehicle, a compound, a nanoparticle composition, nanoparticles (or lipid nanoparticles) in the manufacture of a medicament for the treatment or prophylaxis of a disease, disorder or condition and/or a method of treatment or prophylaxis of a disease, disorder or condition, comprising a step of administering (e.g. in a therapeutically effective amount of) said carrier, a nanocarrier, a delivery system/vehicle, a compound, a nanoparticle composition, nanoparticles (or lipid nanoparticles) to a subject (e.g., vertebrate such as a human or a large veterinary mammal (e.g., horses, cattle, deer, sheep, llamas, goats, pigs) in need thereof.

The disease, disorder or condition may be selected from the group consisting of infectious/contagious diseases, viral infections (i.e. diseases caused by virus), bacterial infections (i.e. diseases caused by bacteria), fungal infections (i.e. diseases caused by fungi), respiratory diseases or the like, cancer, cardiovascular diseases, skin disease or the like, or combinations thereof. In various embodiments, the disease, disorder or condition is mediated by an influenza virus (e.g., influenza A, B, C and/or D virus). For example, the disease may be influenza A, B, C or D such as H1 N1, H3N2). In various embodiments, the disease, disorder or condition is mediated by a coronavirus (e.g., severe acute respiratory syndrome coronavirus such as SARS-CoV-2 or SARS-CoV-1). For example, the disease, disorder or condition may be SARS-CoV-2 coronavirus disease.

In various embodiments, there is also provided a carrier, a nanocarrier, a delivery system/vehicle, a compound, a nanoparticle composition, nanoparticles (or lipid nanoparticles) disclosed herein for use in encapsulating and/or delivering a therapeutic, prophylactic and/or biological agent to a subject, cell, cytosol, tissue or organ (e.g., a mammalian cell, cytosol, tissue or organ), the use of said carrier, a nanocarrier, a delivery system/vehicle, a compound, a nanoparticle composition, nanoparticles (or lipid nanoparticles) in the manufacture of a medicament for encapsulating and/or delivering a therapeutic, prophylactic and/or biological agent to a subject, cell, cytosol, tissue or organ (e.g., a mammalian cell, cytosol, tissue or organ), and/or a method of delivering a therapeutic, prophylactic and/or biological agent to a subject, cell, cytosol, tissue or organ (e.g., a mammalian cell, cytosol, tissue or organ), comprising a step of administering (e.g. in a therapeutically effective amount of) said carrier, a nanocarrier, a delivery system/vehicle, a compound, a nanoparticle composition, nanoparticles (or lipid nanoparticles) to a subject (e.g., vertebrate such as a human or a large veterinary mammal (e.g., horses, cattle, deer, sheep, llamas, goats, pigs)) in need thereof.

In various embodiments, there is also provided a carrier, a nanocarrier, a delivery system/vehicle, a compound, a nanoparticle composition, nanoparticles (or lipid nanoparticles) disclosed herein for use in inducing an immune response in a subject (e.g., vertebrate such as a human or a large veterinary mammal (e.g., horses, cattle, deer, sheep, llamas, goats, pigs)), the use of said carrier, a nanocarrier, a delivery system/vehicle, a compound, a nanoparticle composition, nanoparticles (or lipid nanoparticles) in the manufacture of a medicament for inducing an immune response in a subject, and/or a method of inducing an immune response in a subject, comprising a step of administering (e.g. in a therapeutically effective amount of) said carrier, a nanocarrier, a delivery system/vehicle, a compound, a nanoparticle composition, nanoparticles (or lipid nanoparticles) to a subject in need thereof. In various embodiments, an immune response in the subject is to be induced through the administration of the compound, a nanoparticle composition, nanoparticles (or lipid nanoparticles) thereto. In various embodiments, by inducing an immune response in the subject, the subject is protected against various diseases, disorders or conditions e.g., infectious/contagious diseases, viral infections (i.e. diseases caused by virus), bacterial infections (i.e. diseases caused by bacteria), fungal infections (i.e. diseases caused by fungi), respiratory diseases or the like, or combinations thereof as mentioned herein. The carrier, nanocarrier, delivery system/vehicle, compound, nanoparticle composition, nanoparticles may be delivered to a subject in the form of or as a component of a vaccine.

In various embodiments, the disease, disorder or condition is mediated by an influenza virus (e.g., influenza A, B, C and/or D virus). For example, the disease may be influenza A, B, C or D such as H1N1, H3N2). In various embodiments, the disease, disorder or condition is mediated by a coronavirus (e.g., severe acute respiratory syndrome coronavirus such as SARS-CoV-2 or SARS-CoV-1). For example, the disease, disorder or condition may be SARS-CoV-2 coronavirus disease.

In various embodiments, the carrier, nanocarrier, delivery system/vehicle, compound, nanoparticle composition, nanoparticles prepared from embodiments of the method disclosed herein comprises one or more of the following characteristics or properties: broad applicability (e.g., can be used to encapsulate, deliver and/or transfect a wide range of therapeutic, prophylactic and/or biological reagents), nanosized, substantially neutral surface charge or negative surface charge, high encapsulation efficiency (e.g., >80%), high transfection efficiency (e.g., >80%), high stability, low toxicity (e.g., low cytotoxicity), low production/synthesis cost, therefore making them suitable for in vivo applications that require efficient cellular uptake and/or gene transfection.

### BRIEF DESCRIPTION OF FIGURES

FIG. 1 shows ¹H NMR spectrum of L-Glu-NCA (solvent, DMSO-d₆) in accordance with various embodiments disclosed herein.

FIG. 2 shows ¹³C NMR spectrum of L-Glu-NCA (solvent, DMSO-d₆) in accordance with various embodiments disclosed herein.

FIG. 3 shows ¹H NMR spectrum of tert-butyl (2-(ditetradecylamino)-2-oxoethyl)carbamate (solvent, CDCl₃) in accordance with various embodiments disclosed herein.

FIG. 4 shows ¹H NMR spectrum of 2-amino-N,N-ditetradecylacetamide (solvent, CDCl₃) in accordance with various embodiments disclosed herein.

FIG. 5 shows ¹H NMR spectrum of lipid-block-poly(y-benzyl-L-Glu)₄₀ (solvent, CD₂C₂) in accordance with various embodiments disclosed herein.

FIG. 6 shows ¹H NMR spectrum of lipid-poly(L-Glu)₄₀ (solvent, DMSO-d₆) 20 in accordance with various embodiments disclosed herein.

FIG. 7 shows ¹H NMR spectrum of lipid-poly(L-Glu)₃₆-graft-(D-mannosamine)₁₆ (solvent, DMSO-d₆) in accordance with various embodiments disclosed herein.

FIG. 8 shows ¹H NMR spectrum of lipid-poly(L-Glu)₄₀-graft-(D-mannosamine)₁₈ (solvent, DMSO-d₆) in accordance with various embodiments disclosed herein.

FIG. 9 shows ¹H NMR spectrum of lipid-poly(L-Glu)₄₀-graft-(D-mannosamine)₂₂ (solvent, DMSO-d₆) in accordance with various embodiments disclosed herein.

FIG. 10 shows ¹H NMR spectrum of lipid-poly(L-Glu)₄₀-graft-(D-mannosamine)₂₈ (solvent, DMSO-d₆) in accordance with various embodiments disclosed herein.

FIG. 11 shows ¹H NMR spectrum of lipid-poly(L-Glu)₄₀-graft-(D-mannosamine)₂₆ (solvent, DMSO-d₆) in accordance with various embodiments disclosed herein.

FIG. 12 shows ¹H NMR spectrum of lipid-poly(L-Glu)₄₀-graft-(D-mannosamine)₈-(ethanolamine)₂₇ (solvent, DMSO-d₆) in accordance with various embodiments disclosed herein.

FIG. 13 shows ¹H NMR spectrum of lipid-poly(L-Glu)₄₀-graft-(D-mannosamine)₁₃-(ethanolamine)₂₂ (solvent, DMSO-d₆) in accordance with various embodiments disclosed herein.

FIG. 14 shows ¹H NMR spectrum of lipid-poly(L-Glu)₄₀-graft-(D-mannosamine)₁₆-(ethanolamine)₁₈ (solvent, DMSO-d₆) in accordance with various embodiments disclosed herein.

FIG. 15 shows ¹H NMR spectrum of lipid-poly(L-Glu)₄₀-graft-(D-mannosamine)₁₀-(ethanolamine)₂₄ (solvent, DMSO-d₆) in accordance with various embodiments disclosed herein.

FIG. 16 shows ¹H NMR spectrum of lipid-poly(L-Glu)₄₀-graft-(D-mannosamine)₁₀-(ethanolamine)₂₁ (solvent, DMSO-d₆) in accordance with various embodiments disclosed herein.

FIG. 17 shows ¹H NMR spectrum of lipid-poly(L-Glu)₄₀-graft-(D-mannosamine)₁₀-(ethanolamine)₂₀ (solvent, DMSO-d₆) in accordance with various embodiments disclosed herein.

FIG. 18A, FIG. 18B and FIG. 18C show an experimental plan for in vivo transfection analysis in accordance with various embodiments disclosed herein.

FIG. 18A shows timepoint for in vivo analysis imaging. FIG. 18B shows in vivo bioluminescence imaging groups. FIG. 18C shows subcutaneous injection site at right foot palm of mice.

FIG. 19 shows viability and mRNA transfection efficiency of HELA cells after 48-h transfection with mRNA LNPs designed in accordance with various embodiments disclosed herein. Statistical significance was determined using the Mann —Whitney test comparing ALC-0159 to other LPP formulations at 0.8% mole ratio (** p<0.01).

FIG. 20 shows viability and mRNA transfection efficiency of HELA cells after 48-h transfection with mRNA LNPs designed in accordance with various embodiments disclosed herein. Statistical significance was determined using the Mann —Whitney test comparing ALC-0159 to other LPP formulations at 1.0% mole ratio (* p<0.05, ** p<0.01).

FIG. 21 shows viability and mRNA transfection efficiency of HELA cells after 48-h transfection with mRNA LNPs designed in accordance with various embodiments disclosed herein. Statistical significance was determined using the Mann —Whitney test comparing ALC-0159 to LPP formulations at 1.2% mole ratio (* p<0.05, ** p<0.01).

FIG. 22 shows viability and mRNA transfection efficiency of HELA cells after 48-h transfection with mRNA LNPs designed in accordance with various embodiments disclosed herein. Statistical significance was determined using the Mann —Whitney test comparing ALC-0159 to LPP formulations at 1.4% mole ratio (* p<0.05, ** p<0.01).

FIG. 23 shows viability and mRNA transfection efficiency of HELA cells after 48-h transfection with mRNA LNPs designed in accordance with various embodiments disclosed herein. Statistical significance was determined using the Mann —Whitney test comparing ALC-0159 to LPP formulations at 1.6% mole ratio (* p<0.05, ** p<0.01).

FIG. 24 shows viability and mRNA transfection efficiency of RAW264.7 cells after 48-h transfection with LPP2 made from LPP2 of 1.2% mole content, in accordance with various embodiments disclosed herein. Statistical significance was determined using the Mann —Whitney test comparing ALC-0159 to LPP2 (** p<0.01).

FIG. 25A and FIG. 25B shows in vivo imaging of the dorsal view of Balb/c mice at the 6 h time point, in accordance with various embodiments disclosed herein. Regions of interest at the right foot where luminescence was observed are circled. In FIG. 25A, from left to right, mice 1 is the control, and mice 2 and 3 is were injected with mRNA ALC-0159 LNPs (mRNA dose: 400 ng). In FIG. 25B, from left to right, mice 1 is the control, and mice 2 and 3 were injected with mRNA LPP2 LNPs (LPP2:1.2%, mRNA dose: 400 ng).

FIG. 26A and FIG. 26B shows in vivo imaging of the dorsal view of Balb/c mice at the 24 h time point, in accordance with various embodiments disclosed herein. Regions of interest at the right foot where luminescence was observed are circled. In FIG. 26A, from left to right, mice 1 is the control, and mice 2 and 3 were injected with mRNA ALC-0159 LNPs (mRNA dose: 400 ng). In FIG. 26B, from left to right, mice 1 is the control, and mice 2 and 3 were injected with 400 ng of mRNA LPP2 LNPs (LPP2:1.2%; mRNA dose: 400 ng).

FIG. 27 shows qualitative representation of total flux from regions of interest at the right foot during the 6 h timepoint through the dorsal view, in accordance with various embodiments disclosed herein.

FIG. 28 shows qualitative representation of total flux from regions of interest at the right foot during the 24 h timepoint through the dorsal view, in accordance with various embodiments disclosed herein.

### EXAMPLES

Example embodiments of the disclosure will be better understood and readily apparent to one of ordinary skill in the art from the following examples, tables and if applicable, in conjunction with the figures. It should be appreciated that other modifications related to structural, and/or chemical changes may be made without deviating from the scope of the invention. Example embodiments are not necessarily mutually exclusive as some may be combined with one or more embodiments to form new example embodiments. The example embodiments should not be construed as limiting the scope of the disclosure.

The following examples describe the development of a series of lipid-polypeptides (LPPs) that are useful for delivery of a therapeutic and/or prophylactic agent and/or biological agent (e.g., a nucleic acid such as mRNA, siRNA, pDNA, ASOs etc). Advantageously, embodiments of the LPPs disclosed herein are designed to replace/substitute conventional PEG-lipid conjugate such as ALC-0159. In the following examples, the polypeptide block in the LPPs is functionalized with mannose groups for targeting mannose receptors on immune cell surface to enhance vaccination efficacy. Advantageously, mRNA LNPs encapsulated using the LPPs designed in accordance with various embodiments disclosed herein showed a good size distribution (PDI<0.2) and comparable/greater encapsulation efficiencies compared to LNPs of the art that are formulated with a conventional PEG-lipid conjugate ALC-0159 (e.g., such as that currently used in Pfizer/BioNTech mRNA LNP vaccine). Advantageously, transfection efficiencies of mRNA LNPs formulated with LPPs at varying optimal compositions designed in accordance with various embodiments disclosed herein are higher than those of mRNA LNPs formulated with a conventional PEG-lipid conjugate ALC-0159. Even more advantageously, mRNA translation efficiency of the mannose-functionalized mRNA LPP LNPs in accordance with various embodiments has shown to be tremendously higher in immune cells as compared to those of mRNA LNPs formulated with a conventional PEG-lipid conjugate ALC-0159. Through an in vivo study, mRNA LPP LNPs in accordance with various embodiments disclosed herein show comparable transfection efficiency to ALC-0159 mRNA LNPs of the art. As shown in the following examples, the LPPs of the present application can serve as a viable replacement for ALC-0159 in the delivery of mRNA, potentially reducing the risk of allergic reaction by the PEG-conjugated lipids, prolonging the plasma half-life of mRNA LNPs and enhancing vaccination efficacy.

### Example 1: Materials and Methods

### 1.1. Materials

Chemical reagents for the synthesis of the lipids were purchased from Sigma-Aldrich and used as received unless otherwise noted. 1,2-Distearoyl-sn-glycerol-3-phosphocholine (DSPC), cholesterol, and ALC-0315 were purchased from MedChemExpress (Monmouth Junction, NJ, USA). Sodium acetate was purchased from Sigma-Aldrich (St. Louis, MO, USA). Triton®-X100, Tris-EDTA, and VivoGlo Luciferin, In Vivo Grade, were purchased from Promega (Madison, WI, USA). Alamar Blue and Pierce Firefly Luciferase Glow assay kit were purchased from Invitrogen (Waltham, MA, USA). Other reagents used were of analytical grade.

### 1.2. Synthesis of y-Benzyl-L-Glutamate-N-Carboxyanhydride (L-Glu-NCA)

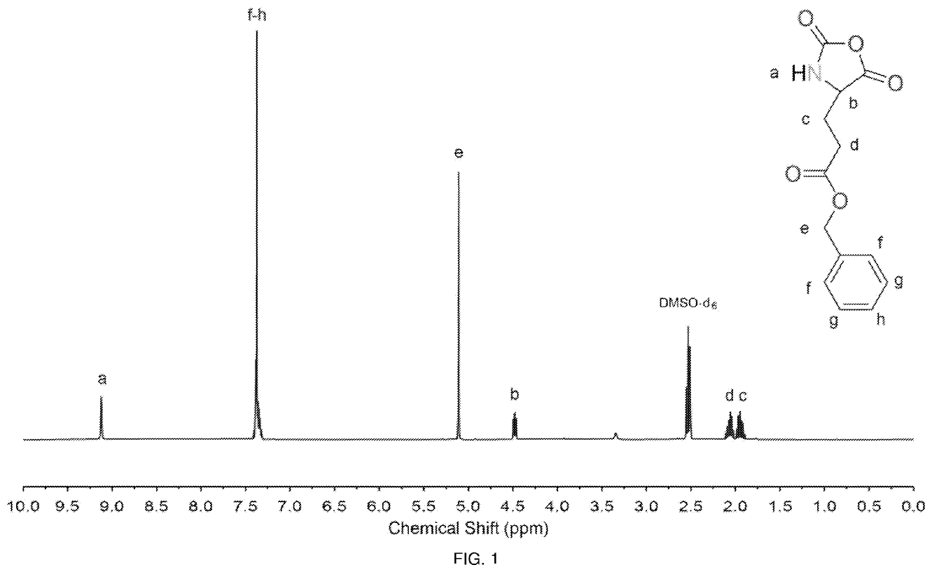

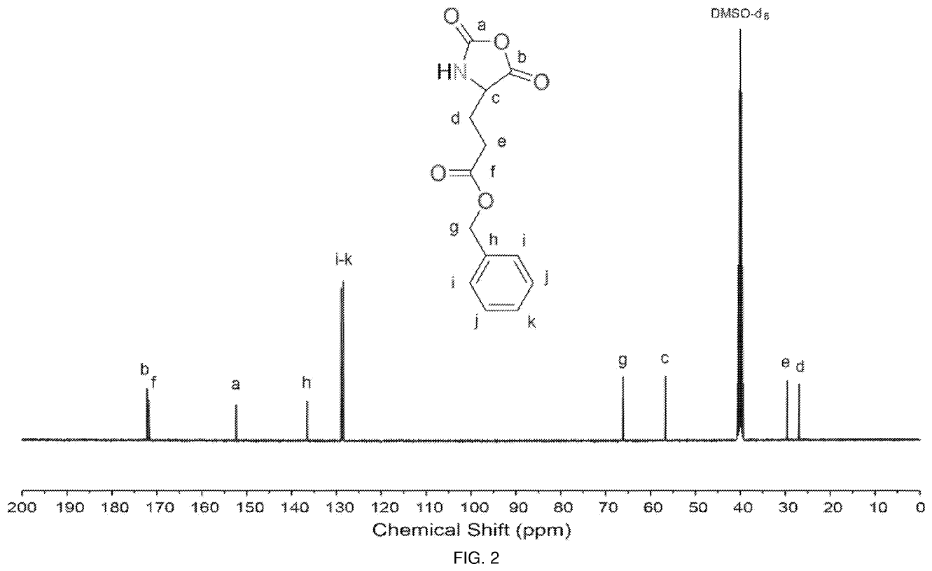

Synthesis strategy for the L-Glu-NCA is showed in Scheme 1. General synthetic method for L-Glu-NCA: γ-benzyl-L-glutamate (6.0 g, 2.53 mmol) was suspended in 100 ml of dry tetrahydrofuran (THF) and then triphosgene (3.4 g) was added under N₂. The mixture was stirred at 70° C. under a flow of N₂ for 3 h. After the reaction mixture was cooled down to room temperature, the crude product was precipitated by pouring the mixture solution into hexane (800 ml), collected by filtration. The resulting crude product was purified by recrystallizing with THF/hexane mixture for three times. The yield of L-Glu-NCA was 70%. The structure of L-Glu-NCA was verified by ¹H NMR and ¹³C NMR spectra (FIG. 1 and FIG. 2 respectively).

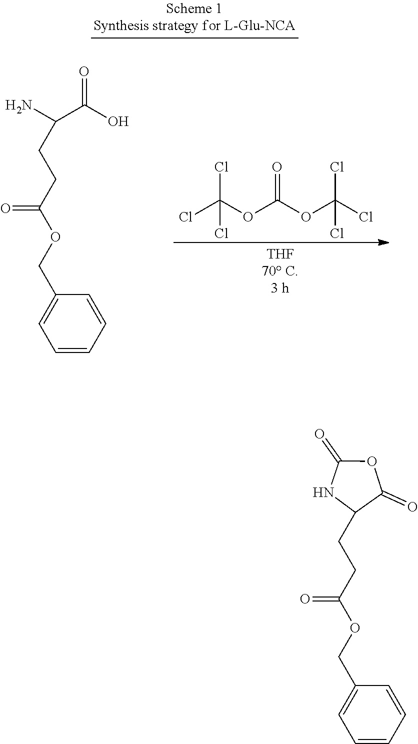 [SMILES: NC(CCC(=O)OCc1ccccc1)C(=O)O ; O=C(OC(Cl)(Cl)Cl)OC(Cl)(Cl)Cl ; O=C(CCC1NC(=O)OC1=O)OCc1ccccc1]

### 1.3. Synthesis of the lipid of 2-amino-N, N-ditetradecylacetamide

Synthesis strategy for the lipid of 2-amino-N,N-ditetradecylacetamide is showed in Scheme 2. tert-Butyl (2-(ditetradecylamino)-2-oxoethyl)carbamate was first synthesized. Boc-glycine (350.3 mg, 2.0 mmol) and ditetradecylamine (819.6 mg, 2.0 mmol) were dissolved in dry dichloromethane (DCM) (40 ml). 2-(1 H-Benzotriazole-1-yl)-1,1,3,3-tetramethyluronium hexafluorophosphate (HBTU) (910.5 mg, 2.4 mmol) and N-ethyl-N-(1-methylethyl)-2-propanamine (DIPEA) (646.1 mg, 5.0 mmol) were added to the solution. The reaction mixture was stirred for 24 h at room temperature under N₂. The mixture was diluted with 50 mL of DCM, and washed with 7% citric acid, brine and H₂₀. The resulting organic layer was collected and dried over anhydrous MgSO₄. DCM was evaporated in vacuo to yield crude product. The resulting crude product was purified by flash silica gel column chromatography (Hexane:Diethylether 8:2, v/v), obtaining the final product as a yellow oil. The yield of the compound was 85%.

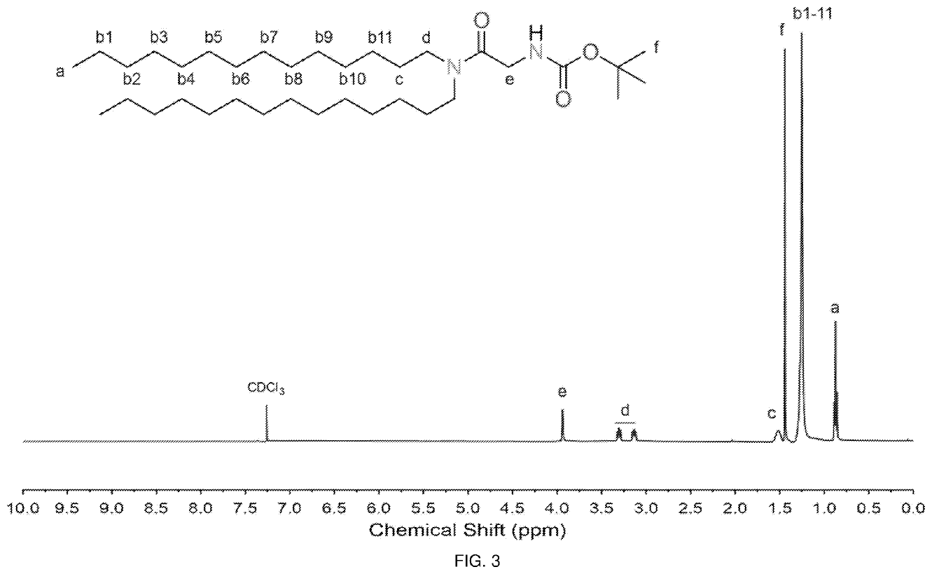

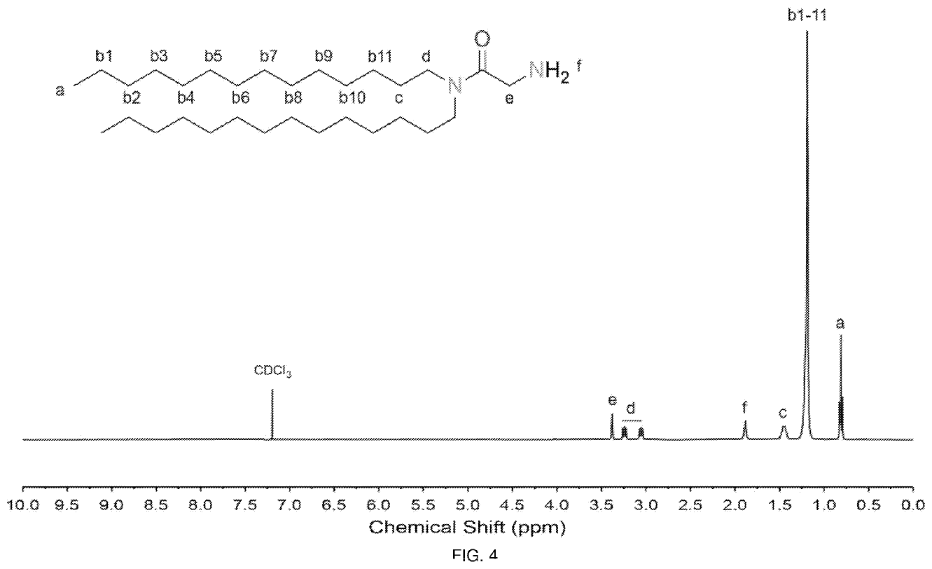

The structure of product was verified by ¹H NMR spectrum (FIG. 3). 2-Amino-N,N-ditetradecylacetamide was then synthesized. tert-Butyl (2-(ditetradecylamino)-2-oxoethyl)carbamate was dissolved in 5 mL of anhydrous DCM, to which was added 4 mL of trifluoroacetic acid (TFA). The mixture was stirred for 2 h under N₂ atmosphere. The solvents were evaporated in vacuo to yield crude product. The resulting crude product was dissolved in 20 mL of DCM, and then 20 mL of NaHCO₃ aqueous solution (10%) was added. The mixture was stirred for 12 h under N₂ atmosphere. The organic layer was collected and dried over anhydrous MgSO₄. The DCM was evaporated in vacuo. The resulting product was dried under vacuum to obtain a white powder. The yield of the compound was 95%. The structure of product was verified by ¹H NMR spectrum (FIG. 4).

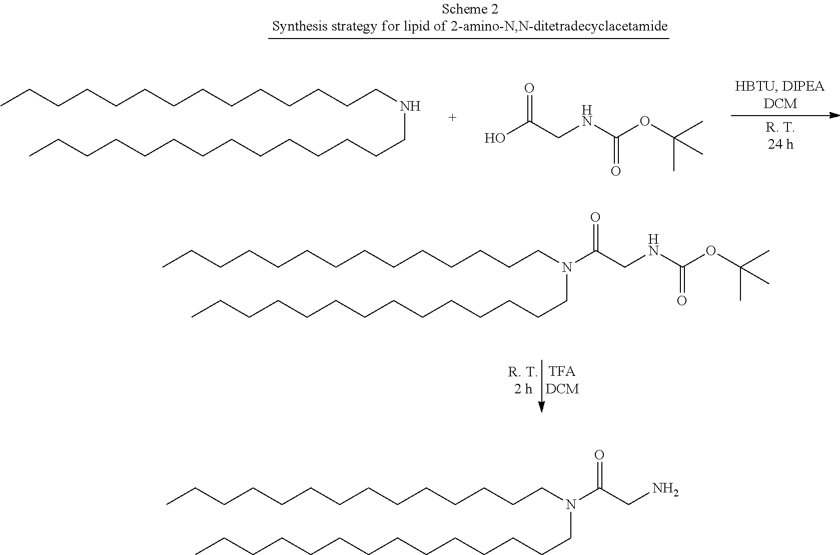

### 1.4. Synthesis of lipid-block-poly(L-Glu)

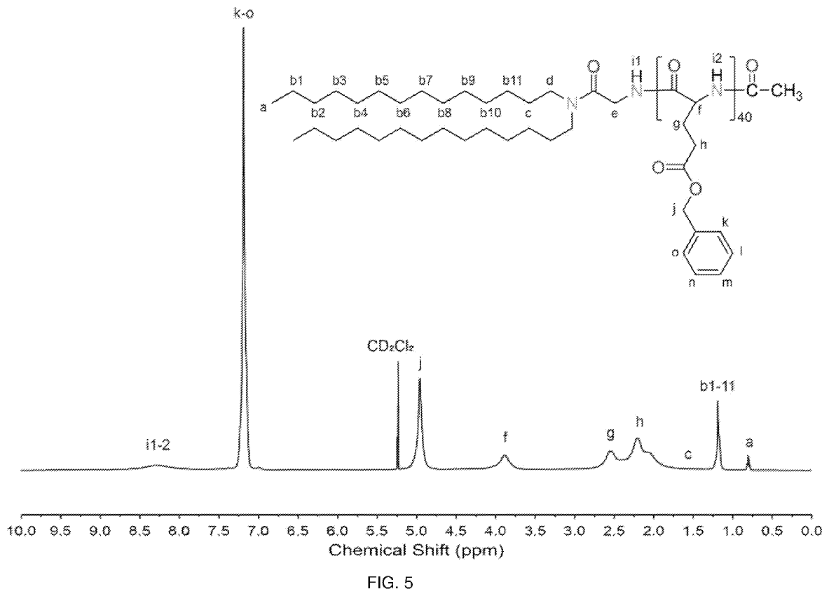

Synthesis strategy for lipid-block-poly(L-Glu) is showed in Scheme 3. Lipid-block-poly(γ-benzyl-L-Glu) was first synthesized. General synthetic method for lipid-poly(γ-benzyl-L-Glu): In the glove box, 2-amino-N, N-ditetradecylacetamide (46.6 mg, 0.1 mmol) and L-Glu-NCA were dissolved in 40 mL of anhydrous DCM. The mixture was stirred for 48 h at room temperature in the glove box. Then, 3.0 mL of acetic anhydride was added and the reaction was continued for 2 h. The crude product was precipitated by pouring the mixture solution into glacial ether (300 ml), collected by centrifugation. The resulting crude product was purified by dissolving with DCM and precipitated by pouring the solution into glacial ether. The resulting product was dried under vacuum. The number of polymerization unit for lipid-poly(γ-benzyl-L-Glu) can be adjusted by varying the amount of L-Glu-NCA. The yield of the protected polymer was 72%. The typical structure of lipid-poly(γ-benzyl-L-Glu)₄₀ was verified by ¹H NMR (FIG. 5).

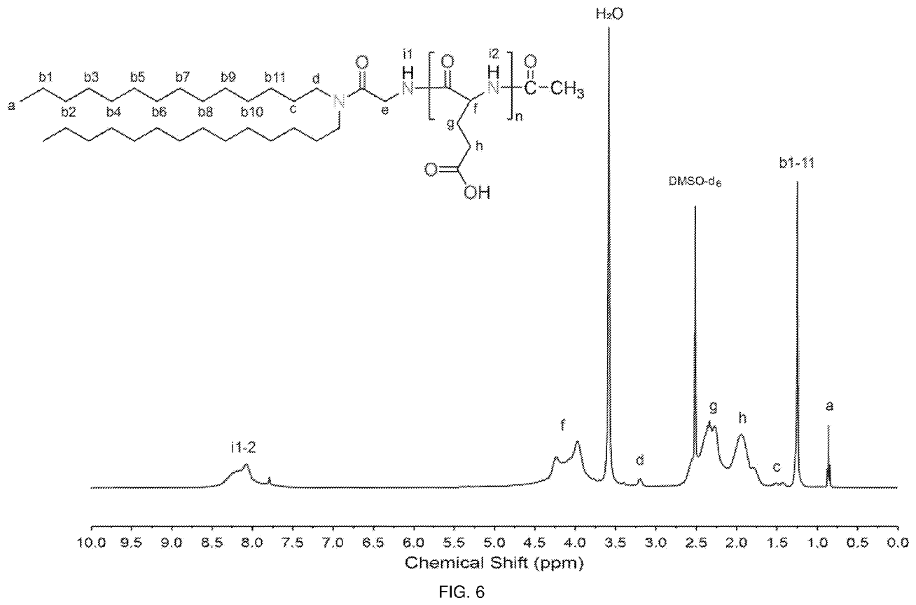

The deprotection of lipid-block-poly(γ-benzyl-L-Glu) was then performed to yield lipid-poly(L-Glutamate) [lipid-poly(L-Glu)]. General synthetic method for lipid-poly(L-Glu): 1.0 g of lipid-poly(γ-benzyl-L-Glu) was dissolved in 10 mL of TFA, to which was added 3 mL of 33% HBr in acetic acid. The mixture was stirred in ice bath for 2 h. The solvents were removed in vacuo. The resulting crude product was suspended in 30 ml of methanol and then precipitated by pouring the mixture solution into glacial ether (300 ml), collected by centrifugation. The crude product was purified by suspending it in methanol and precipitated by pouring the solution into glacial ether. The obtained product was dried under vacuum. The yield of the deprotected polymer was 95%. The typical structure of lipid-poly(L-Glu)₄₀ was verified by ¹H NMR (FIG. 6).

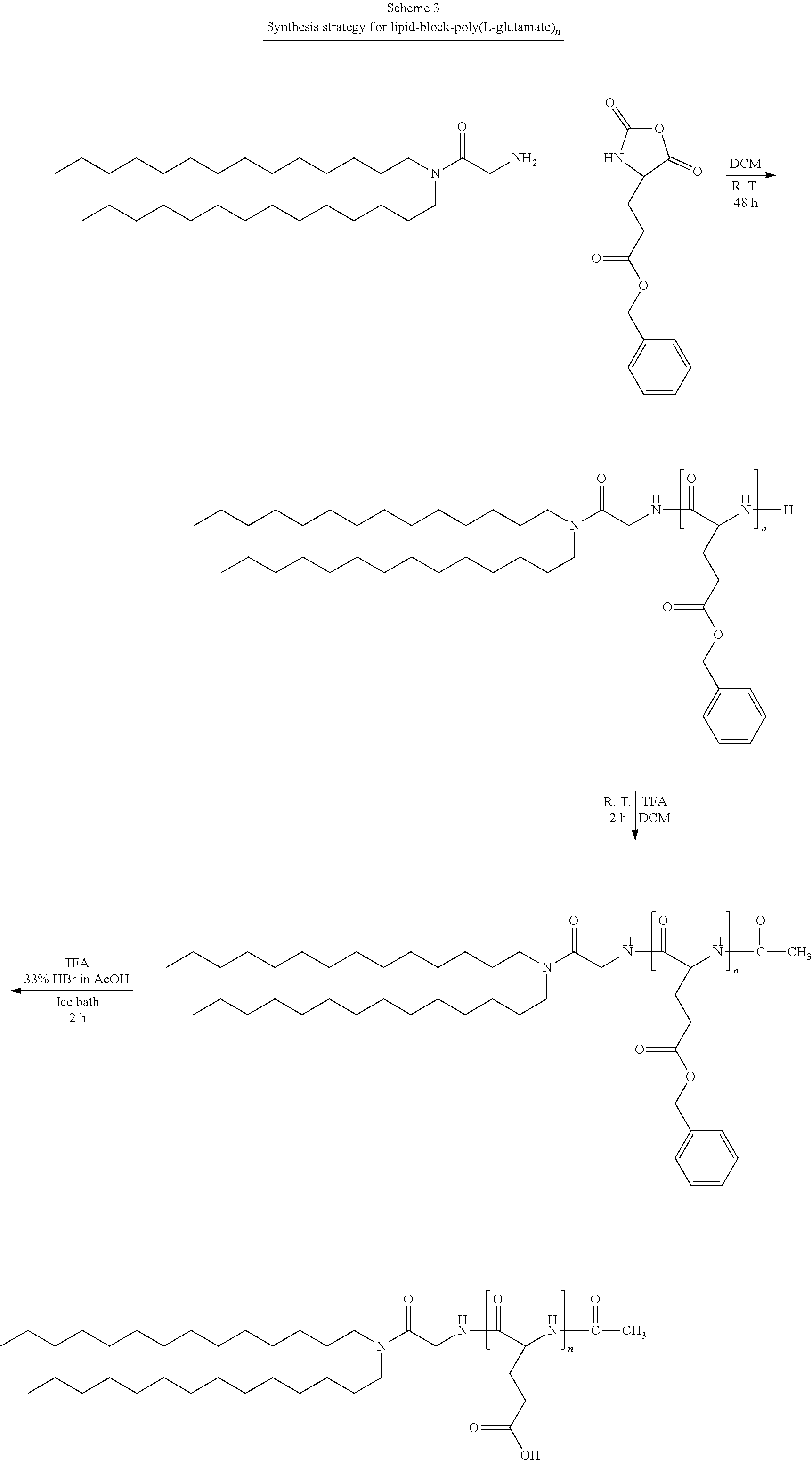

### 1.5. Synthesis of lipid-poly(L-Glu)-raft-(D-mannosamine)

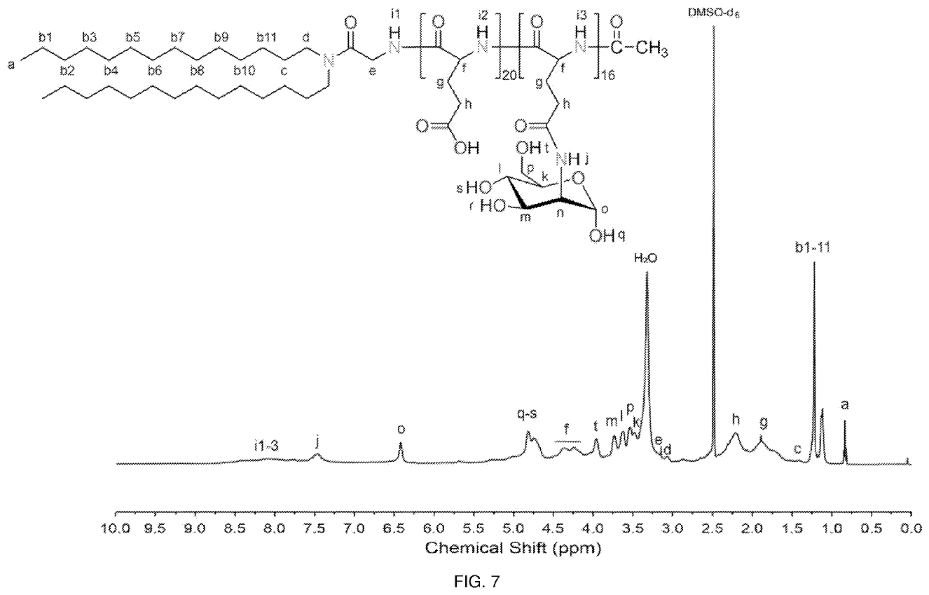

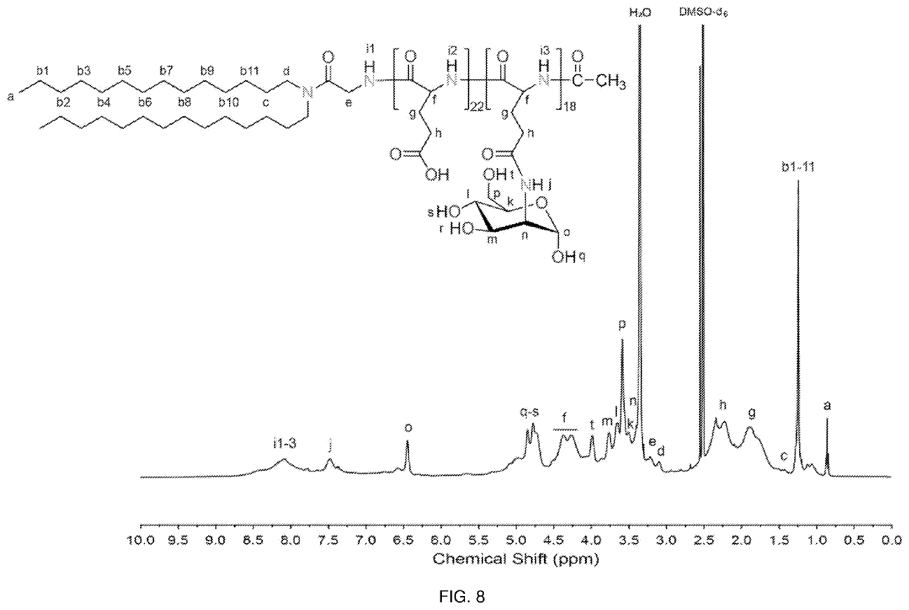

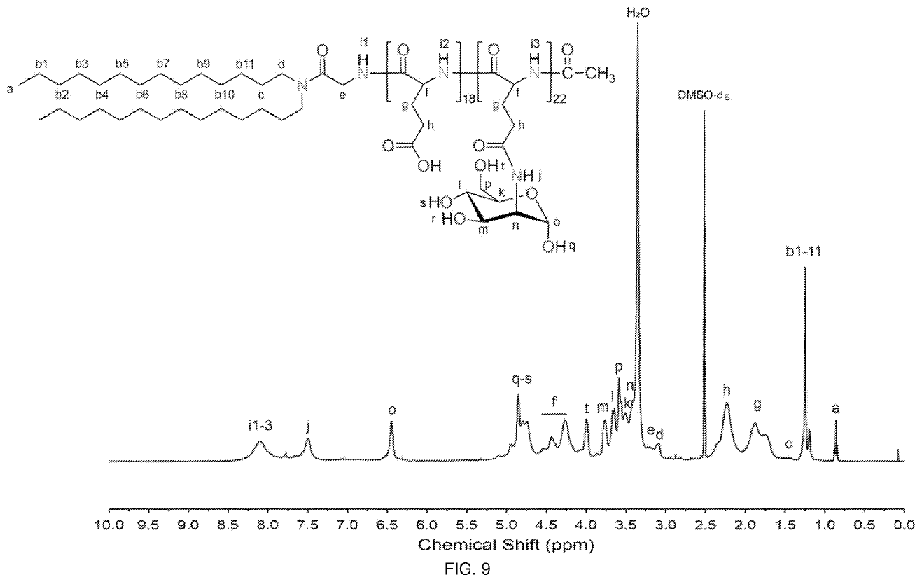

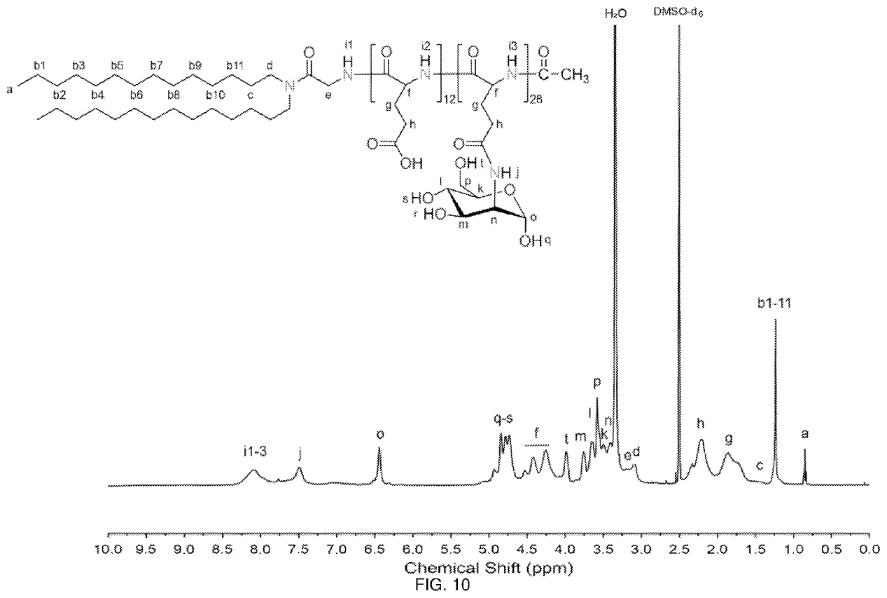

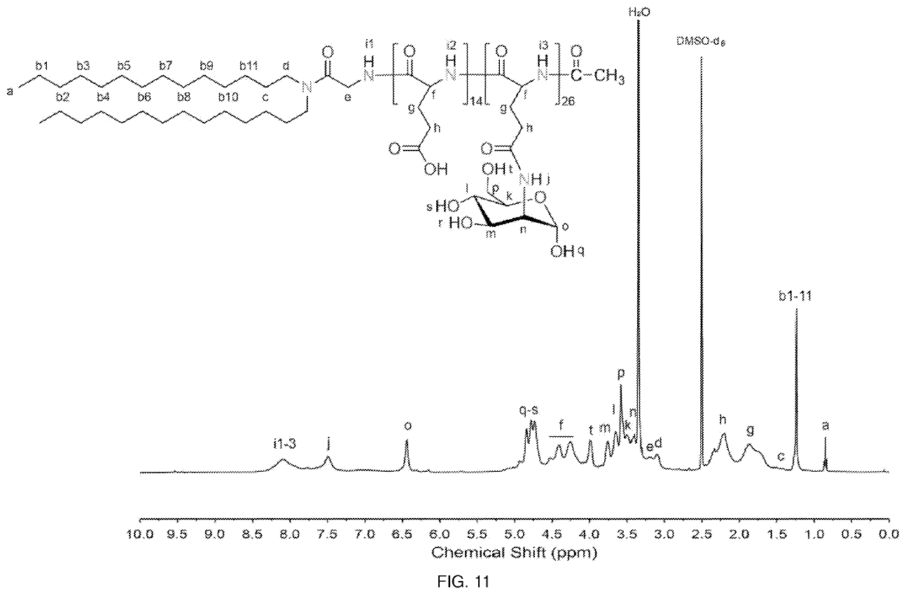

Synthesis strategy for lipid-poly(L-Glu)-graft-(D-mannosamine) is showed in Scheme 4. General synthetic method for lipid-poly(L-Glu)-graft-(D-mannosamine): Lipid-poly(L-Glu) containing 1.0 mmol of L-Glutamate units and D-mannosamine hydrochloride each were dissolved in 10 ml of dry dimethyl sulfoxide (DMSO). HBTU (456 mg, 1.2 mmol) and DIPEA (388 mg, 3.0 mmol) were added to the solution. The reaction mixture was stirred for 12 h at 70° C. under N₂. The reaction mixture was cooled down to room temperature, to which was added 10 mL of HCl aqueous solution (5%). The mixture was stirred for 30 min at room temperature. The crude product was purified by dialysis with deionized water. The product was dried under vacuum. The product was obtained by freeze-drying under vacuum. The amount of D-mannosamine grafted onto lipid-poly(L-Glu) can be varied by changing the substrate feed ratio. A series of polymer structures of lipid-poly(L-Glu)-graft-(D-mannosamine) were verified by ¹H NMR (FIG. 7, FIG. 8, FIG. 9, FIG. 10 and FIG. 11).

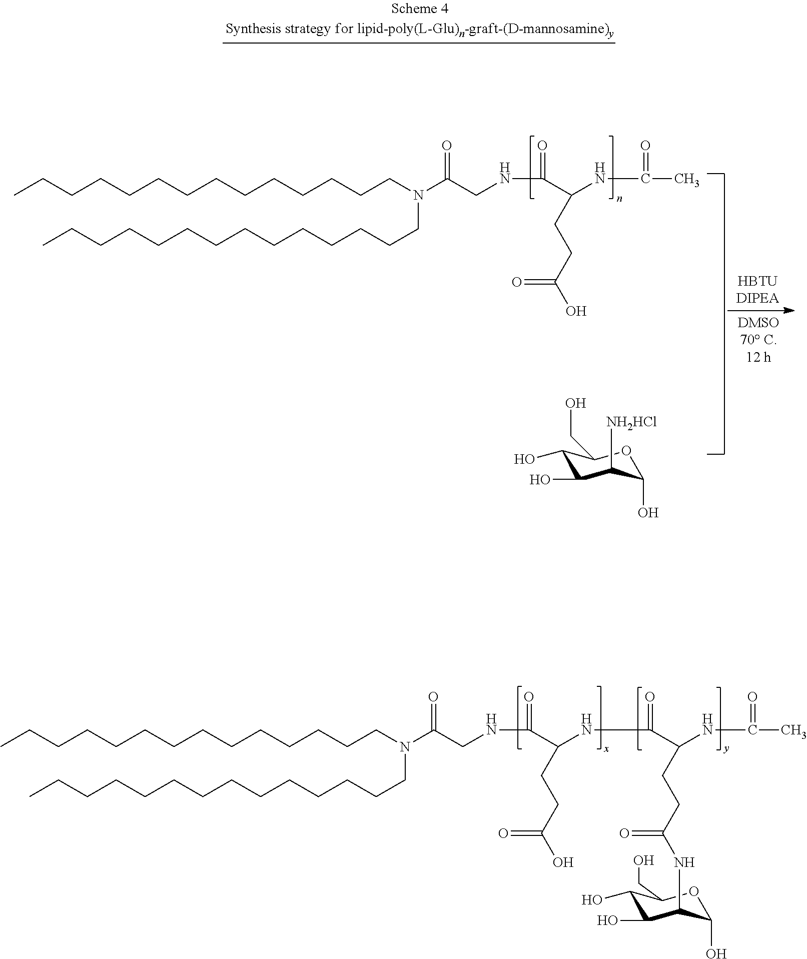

### 1.6. Synthesis of lipid-poly(L-Glu)-raft-(D-mannosamine)-(ethanolamine)

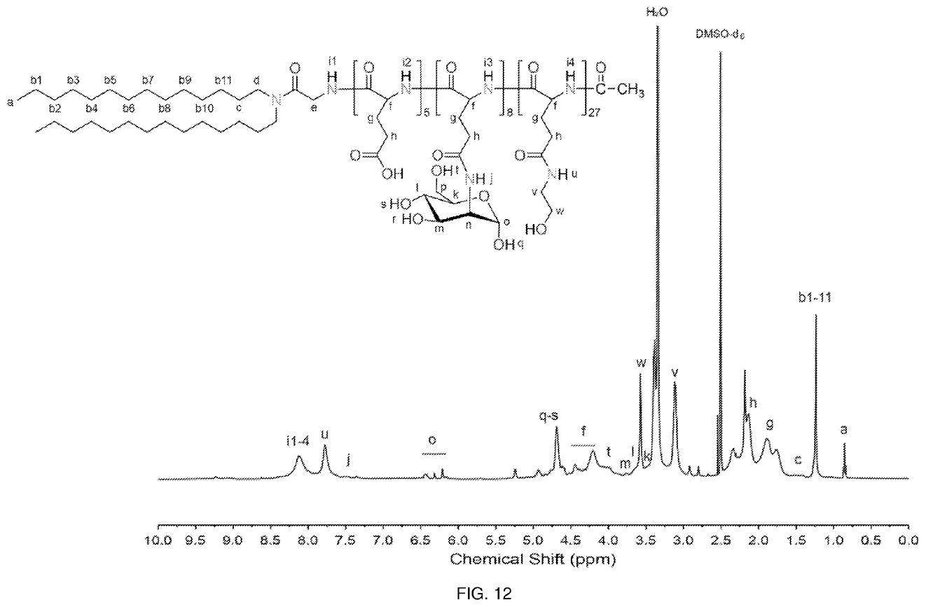

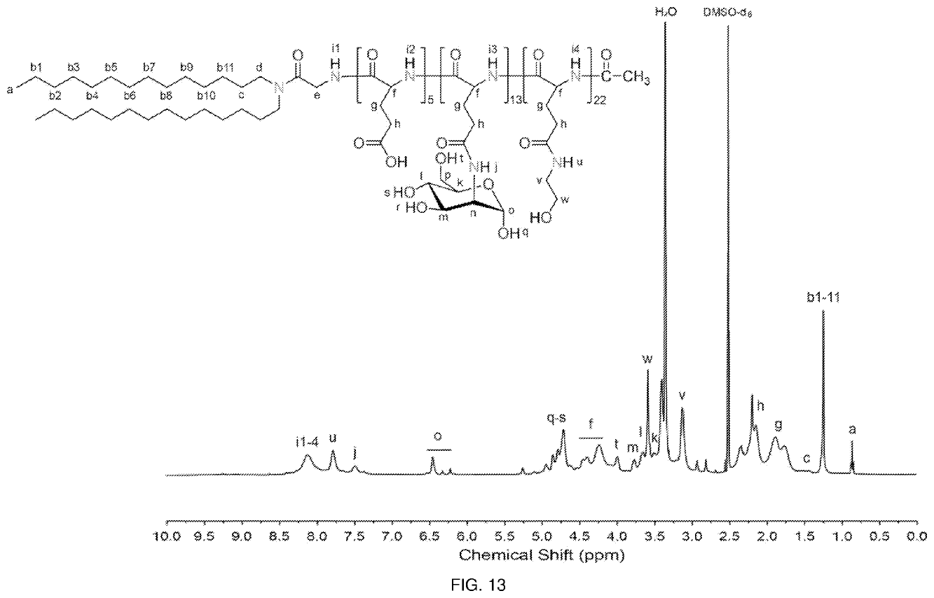

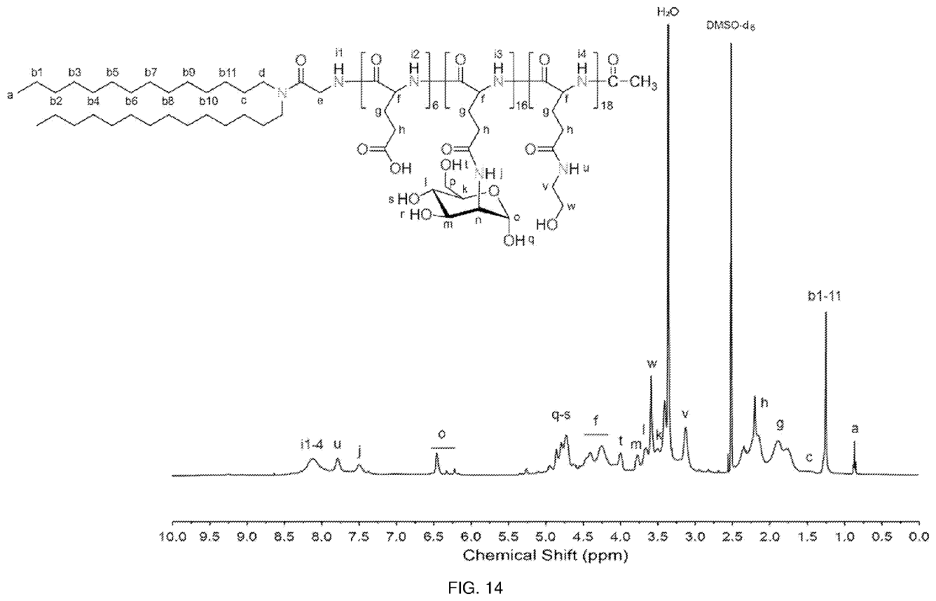

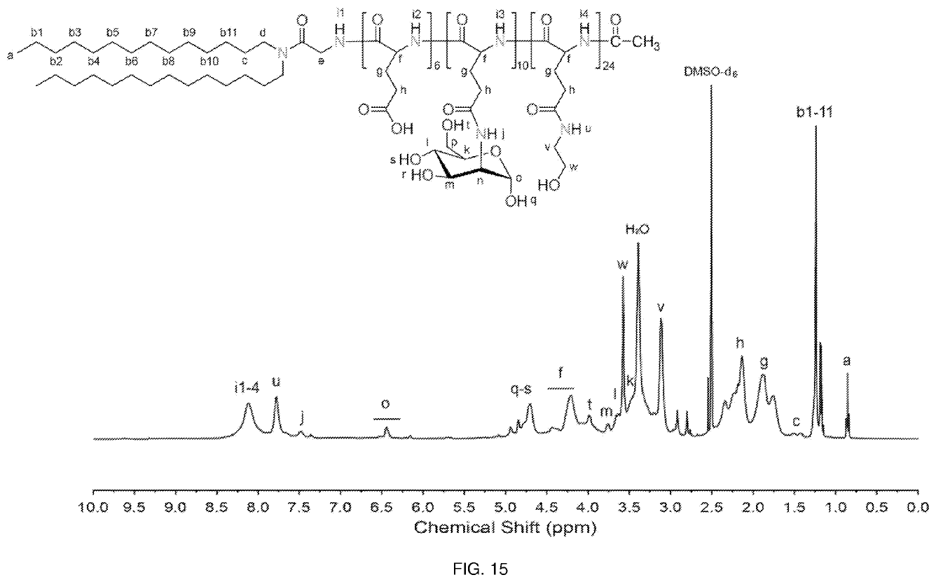

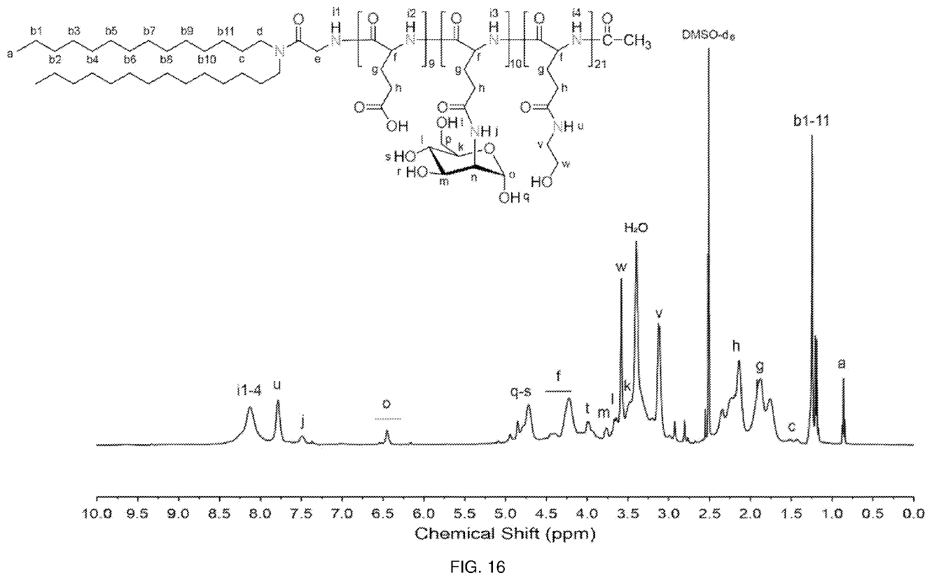

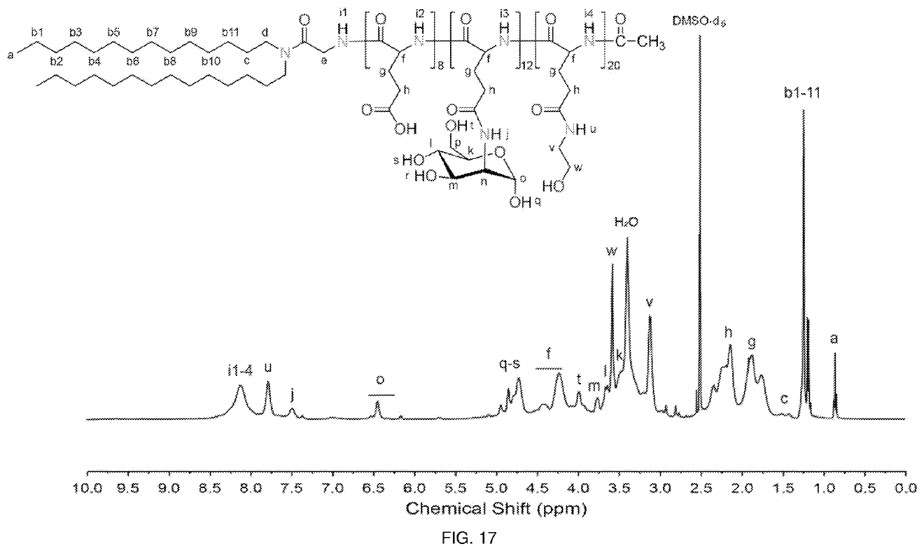

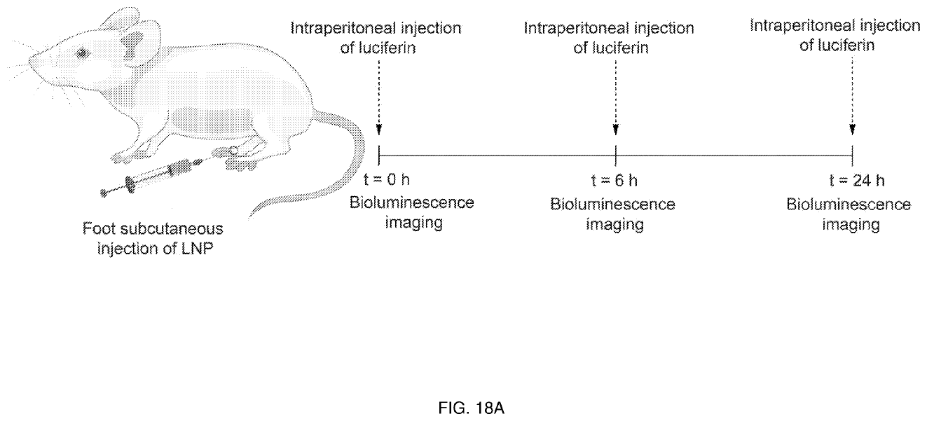

Synthesis strategy for lipid-poly(L-Glu)-graft-(D-mannosamine)-(ethanolamine) is showed in Scheme 5. General synthetic method for lipid-poly(L-Glu)-graft-(D-mannosamine)-(ethanolamine): Lipid-poly(L-Glu) containing 1.0 mmol of L-Glutamate units, D-mannosamine hydrochloride and ethanolamine each were dissolved in 10 ml of dry dimethyl sulfoxide (DMSO). HBTU (456 mg, 1.2 mmol) and DIPEA (388 mg, 3.0 mmol) were added to the solution. The reaction mixture was stirred for 12 h at 70° C. under N₂. The reaction mixture was cooled to room temperature, to which was added 10 mL of HCl aqueous solution (5%). The mixture was stirred for 30 min at room temperature. The crude product was purified by dialysis with deionized water. The product was dried under vacuum. The product was obtained by freeze-drying under vacuum. The amount of D-mannosamine and ethanolamine grafted onto lipid-poly(L-Glu) can be varied by changing the substrate feed ratio. A series of polymer structures of lipid-poly(L-Glu)-graft-(D-mannosamine)-(ethanolamine) were verified by ¹H NMR (FIG. 12, FIG. 13, FIG. 14, FIG. 15, FIG. 16 and FIG. 17).

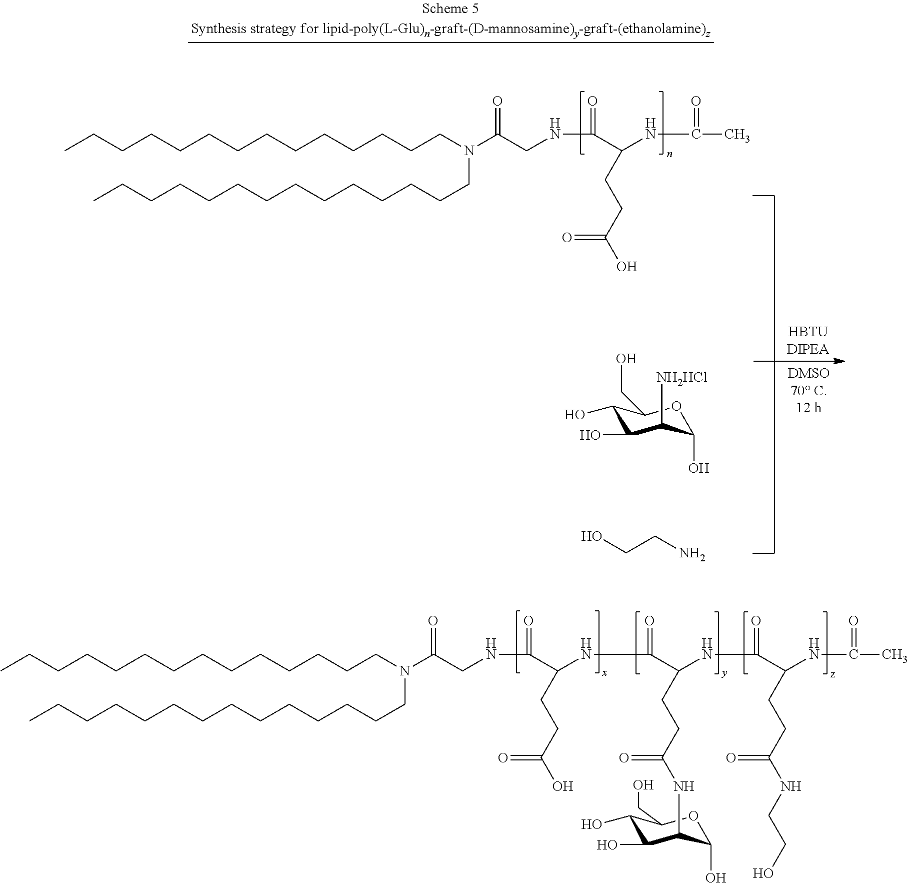

### 1.7. Formulation of mRNA-loaded lipid nanoparticles (mRNA LNPs)

mRNA LNPs were prepared manually with reference to the mole ratio as indicated in Table 1. mRNA weight used per formulation was standardized at 10 μg. The amount of lipids added to the mRNA solution was adjusted to achieve an N/P ratio of 6.

The lipid-polypeptides (LPPs) are made to replace PEG-lipid such as ALC-0159, the mole ratios were varied to determine the optimal mole ratio for each LPP. The different molecular weights of the LPPs are detailed in Table 2. Subsequently, the difference between the mole ratio of LPP and the mole ratio of ALC-0159 (1.6%) was divided among the other lipids as per their mole ratio.

In the organic phase, ethanol was added to the mixture of lipids to reach a final volume of 50 μL. In the aqueous phase, 140 μL of 10 mM sodium acetate solution, pH 4, was added to 10 μL of 1 mg/mL firefly luciferase mRNA (Trilink Biotechnologies) to reach a final volume of 150 μL. The mixture of lipids in the organic phase were directly added into the aqueous solution and mixed rapidly through pipetting. The mRNA LNP solution was then incubated at room temperature for 30 minutes to provide time for encapsulation/self-assembly.

TABLE 1
Mole ratio of lipids used in formulations

| | ALC- | | | ALC- | |
|---|---|---|---|---|---|
| Lipid | 0315 | DSPC | Cholesterol | 0159 | LPP |
| Molecular weight | 766.3 | 790.2 | 386.7 | 2481.0 | Refer to |
| (g/mol) | | | | | Table 2 |
| Mole ratio - | 46.3 | 9.4 | 42.7 | 1.6 | — |
| ALC-0159 (%) | 46.7 | 9.5 | 43.1 | | 0.8% |
| | 46.6 | 9.5 | 42.0 | | 1.0% |
| Mole ratio - | 46.5 | 9.4 | 42.9 | — | 1.2% |
| LPP (%) | 46.4 | 9.4 | 42.8 | | 1.4% |
| | 46.3 | 9.4 | 42.7 | | 1.6% |
| Stock concentration | 20.0 | 10.0 | 10.0 | 3.0 | 10.0 |
| (mg/mL) | | | | | |

TABLE 2
Molecular weights of LPPs

| LPP | Molecular weight (g/mol) |
|---|---|
| LPP1 | 22817.7 |
| LPP2 | 7731.2 |
| LPP3 | 10526.2 |
| LPP4 | 5657.4 |
| LPP9 | 8569.4 |
| LPP10 | 9213.7 |
| LPP11 | 9535.8 |
| LPP12 | 10185.9 |

### 1.8. Assessing Encapsulation Efficiency

Encapsulation efficiencies and mRNA concentration of the formulated mRNA LNPs were determined using the Quant-it™ RiboGreen RNA Assay Kit (Invitrogen, Waltham, MA, USA). RiboGreen RNA reagent was diluted 200 times with Tris-EDTA buffer or 5% Triton-X100 in Tris-EDTA buffer. 90 μL of buffer mixture containing the RiboGreen reagent was added to 10 μL of the varying mRNA LNP solutions and incubated at 37° C. for 20 min. The fluorescence intensity was then recorded using a microplate reader (Tecan, Mannedorf, Switzerland) at an excitation wavelength of 485 nm and an emission wavelength of 520 nm. The fluorescence intensity values were used to obtain the mRNA concentration through an mRNA standard curve. Subsequently, the encapsulation efficiencies of the mRNA LNPs were calculated using the concentration values as follows.

$$Encapsulation Efficiency = \frac{{Conc}_{Trit} - {Conc}_{TE}}{{Conc}_{Trit}} \times 100$$

where Conc_{Trit} is the concentration of the mRNA LNPs obtained by adding to 5% Triton-X100 diluted in Tris-EDTA buffer while ConC_{TE} is the concentration of the respective mRNA LNPs obtained by adding it to Tris-EDTA buffer without Triton-X100.

1.9. Characterization of mRNA LNPs

mRNA LNPs were characterized through a Zetasizer (Malvern, UK). The size and polydispersity index (PDI) of the mRNA LNPs were obtained using dynamic light scattering (DLS) by diluting 25 μL of the freshly made mRNA LNPs with saline to a final volume of 500 μL. The samples were measured three times at 25° C. with 20 runs each time, and the samples were measured at 1.68 s per run. The surface zeta potential of the mRNA LNPs were also measured using the Zetasizer by diluting 25 μL of the freshly made mRNA LNPs suspension with saline to a final volume of 1 mL. The samples were measured three times at 25° C. with 20 runs each time the samples were measured.

1.10. Cell Culturing and Dosing of HELA and RAW264.7 Cells with mRNA LNPs

HELA and RAW264.7 cells were cultured in DMEM with 10% Fetal Bovine Serum (FBS) (V/V) and 1% Penicillin/Streptomycin (V/V). The cells were incubated at 37° C. with 5% CO₂ in an incubator (Thermo Fisher, Waltham, MA, USA). For the dosing of cells with mRNA LNPs, the HELA or RAW264.7 cells were seeded into black 96-well plates for evaluation of cytotoxicity or white 96-well plates for evaluation of mRNA transfection efficiency with a seeding density of 10,000 cells per well. After incubating the cells in the 96-well plates for 24 hours, the media was removed from the wells and replaced with fresh media containing the diluted mRNA LNPs to achieve 100 ng of mRNA added to each well. The 96-well plates were then incubated for 48 hours before testing for cell viability and transfection efficiency.

1.11. In Vitro Cell Viability of HELA and RAW264.7 Cells after Incubation with mRNA LNPS

After 48 hours of incubation, the media containing mRNA LNPs was removed from the wells of the 96-well plate and replaced with 100 μL of fresh media containing 10% Alamar Blue reagent. The cells were then incubated for 2 h at 37° C. Fluorescence intensity was measured using a microplate reader (Tecan, Mǎnnedorf, Switzerland) at an excitation wavelength of 570 nm and an emission wavelength of 600 nm. The viability of the respective wells were calculated based on a negative control group that did not receive any treatment.

1.12. In Vitro Transfection Efficiency of mRNA LNPs in HELA and RAW264.7 Cells

Transfection efficiency of mRNA LNPs in HELA or RAW264.7 cells was also measured after 48 hours of incubation by removing media containing mRNA LNPs and replaced with 100 μL of cell lysis buffer/D-luciferin mixture. The mixture contained 50 μL of 2× cell lysis buffer diluted with Phosphate Buffered Saline (PBS) and 50 μL of 100× D-luciferin diluted with Firefly Assay Buffer. The cells were incubated with the mixture for 10 min at 37° C. to allow for cell lysis and signal stabilization. Subsequently, luminescence intensity was read using a microplate reader (Tecan, Mannedorf, Switzerland) at an exposure time of 1000 ms.

1.13. Preparation of mRNA LNP for In Vivo Transfection Study

Due to the promising in vitro results of LPP2 at mole ratio 1.2%, the formulation was chosen for in vivo transfection study of mRNA LNPs. The LPP2 (1.2% mole ratio) and ALC-0159 formulations were prepared through a microfluidics device using the NanoAssemblr® Ignite™ (Precision Nanosystem, California, USA). Volumes used for the formulation of the LNPs are indicated in Table 3. mRNA used for both formulations was standardized at 100 μg.

TABLE 3
Mole ratio of lipids used for in vivo analysis

| | ALC- | | | ALC- | |
|---|---|---|---|---|---|
| Lipid | 0315 | DSPC | Cholesterol | 0159 | LPP2 |
| Molecular weight | 766.3 | 790.2 | 386.7 | 2481.0 | 7731.2 |
| (g/mol) | | | | | |
| Mole ratio - | 46.3 | 9.4 | 42.7 | 1.6 | — |
| ALC-0159 (%) | | | | | |
| Mole ratio - | 46.5 | 9.4 | 42.9 | — | 1.2 |
| LPP2 (%) | | | | | |
| Stock concentration | 20.0 | 10.0 | 10.0 | 3.0 | 10.0 |
| (mg/mL) | | | | | |

1.14. In Vivo Transfection of mRNA LNPs in BALB/c Mice

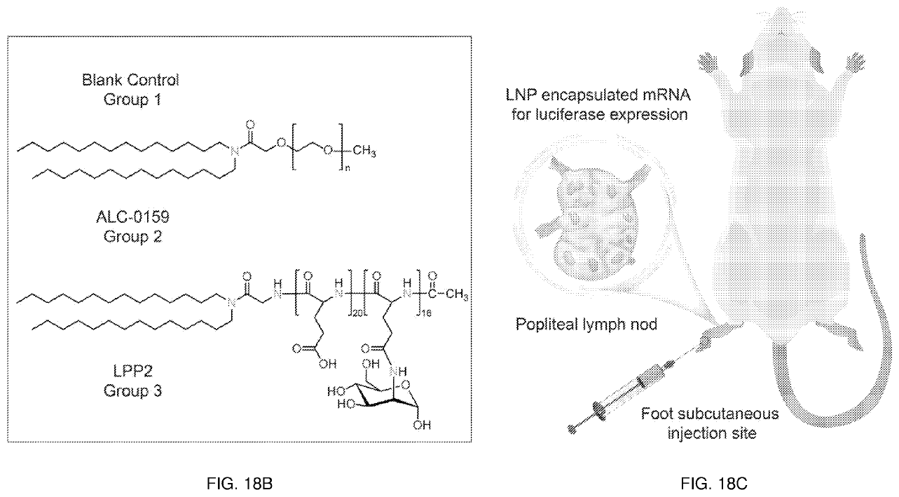

For the in vivo analysis of mRNA transfection, n=2 BALB/c mice were allocated to each group for the ALC-0159 (1.6% mole ratio) group and LPP2 (1.2% mole ratio) group. The negative control consisted of n=1 mouse that was not administered any treatment. 20 μL of 20 ng/μL mRNA LNPs were administered to the right foot palm of the respective groups via subcutaneous injection. 200 μL of 15 mg/mL VivoGlo Luciferin, diluted with PBS, was administered to each mouse through the intraperitoneal route and the signal was allowed to stabilize for 10 min before imaging. At the 6 h and 24 h timepoints, the mice were imaged for their fluorescence signal intensity at exposure times of 10 through the IVIS Lumina XR (Caliper LifeSciences, Massachusetts, USA). The schematic of the in vivo experimental design is represented by FIG. 18A, FIG. 18B and FIG. 18C.

### Example 2: Synthesis of LPPs

As shown in Scheme 3, Lipid-block-poly(L-Glu) was synthesized via the ring-opening polymerization (ROP) of γ-benzyl-L-glutamate-N-carboxyanhydride (Scheme 1) using the lipid of 2-amino-N, N-ditetradecylacetamide (Scheme 2) as the initiator, followed by the deprotection of lipid-block-poly(γ-benzyl-L-Glu) in TFA and HBr/CH₃COOH. The successful synthesis of γ-benzyl-L-glutamate-N-carboxyanhydride, 2-amino-N, N-ditetradecylacetamide and lipid-poly(L-Glu) was verified by ¹H NMR spectroscopy (FIG. 1, FIG. 2, FIG. 3, FIG. 4, FIG. 5 and FIG. 6). As shown in FIG. 6, the disappearance of peaks i-k (—PhH—) and e (Ph—CH₂—) at δ 7.35 and 5.25 ppm, respectively, indicated successful deprotection of lipid-poly(L-Glu). The degrees of polymerization (DPs) of L-Glutamate in lipid-poly(L-Glu) can be adjusted by varying the amount of L-Glu-NCA. The DPs of L-Glutamate in lipid-poly(L-Glu) were determined by the integration area (peaks h and g of the polypeptide; peak a of the lipid) (FIG. 6, FIG. 7). The lipid-poly(L-Glu) with DP of 36 and 40 were first synthesized and denoted as lipid-poly(L-Glu)₃₆ and lipid-poly(L-Glu)₄₀, respectively.

The lipid-poly(L-Glu) has poor solubility in aqueous solution. To increase the hydrophilicity of lipid-poly(L-Glu) and render it with targeting ability towards mannose receptors on immune cells, D-mannosamine group was conjugated to the lipid-polypeptide via the amidation reaction of D-mannosamine with poly(L-Glu). The lipid-poly(L-Glu), with various DPs, was functionalized with D-mannosamine functional group, to obtain a series of hydrophilic lipid-poly(L-Glu)-(D-mannosamine). D-mannosamine-functionalized lipid-poly(L-Glu) was synthesized using D-mannosamine hydrochloride as the amidating agent (Scheme 4). Using lipid-poly(L-Glu)₃₆ and lipid-poly(L-Glu)₄₀ as representative examples, the success of D-mannosamine-functionalization was confirmed by the appearance of peaks o (—CH— in D-mannosamine group) and j (D-mannose-NH—) at δ 6.45 and 7.49 ppm, respectively (FIG. 7, FIG. 8, FIG. 9, FIG. 10 and FIG. 11). The degree of functionalization was determined by the integration area under peaks a, g and o (peak a of the lipid; peak g of the polypeptide; peak o of the D-mannosamine). By varying the D-mannosamine feed, the maximum degree of functionalization for lipid-poly(L-Glu) reached up to 70% (Tables 4, 5).

TABLE 4
Reactant feed ratio of lipid-poly(L-Glu)-(D-mannosamine)

| | Polymer name | | | | |
|---|---|---|---|---|---|
| | | | Acti- | | |
| | Substrate 1 | Substrate 2 | vator | Base | Solvent |
| | Lipid-polypeptide | | | | |
| | lipid-poly(L- | D- | | | |
| | Glu) | mannosamine | HBTU | DIPEA | DMSO |
| | (mg) | (mmol) | (mmol) | (mmol) | (mL) |
| LPP1 | 190 | 1.0 | 1.2 | 3.0 | 10.0 |
| LPP2 | 185 | 1.0 | 1.2 | 3.0 | 10.0 |
| LPP3 | 185 | 1.0 | 1.2 | 3.0 | 10.0 |
| LPP4 | 185 | 1.0 | 1.2 | 3.0 | 10.0 |
| LPP5 | 180 | 0.5 | 1.2 | 3.0 | 10.0 |
| LPP6 | 180 | 1.0 | 1.2 | 3.0 | 10.0 |
| LPP7 | 180 | 2.0 | 1.2 | 3.0 | 10.0 |
| LPP8 | 180 | 3.0 | 1.2 | 3.0 | 10.0 |

TABLE 5
Design, structure and characteristics of lipid-poly(L-Glu)-(D-mannosamine)

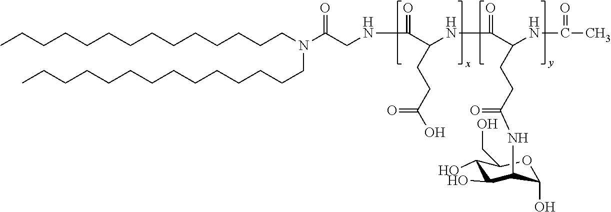

| Polymer | Structure units | | | | |
|---|---|---|---|---|---|
| name | | y | | | |
| Lipid- | x | L-Glu-D- | | Mₙ^{NMR} | Functionalization |
| polypeptide | L-Glu | mannosamine | n | [g mol⁻¹] | degree (%) |
| LPP1 | 50 | 55 | 105 | 22817.7 | 52.4 |
| LPP2 | 20 | 16 | 36 | 7731.2 | 44.4 |
| LPP3 | 24 | 24 | 48 | 10526.2 | 50.0 |
| LPP4 | 11 | 13 | 24 | 5657.4 | 54.2 |
| LPP5 | 22 | 18 | 40 | 8569.4 | 45.0 |
| LPP6 | 18 | 22 | 40 | 9213.7 | 55.0 |
| LPP7 | 14 | 26 | 40 | 9535.8 | 65.0 |
| LPP8 | 12 | 28 | 40 | 10185.9 | 70.0 |

To shield the anionic charge groups, lipid-poly(L-Glu) was functionalized with both D-mannosamine and ethanolamine functional groups, to obtain a series of hydrophilic lipid-poly(L-Glu)-(D-mannosamine)-(ethanolamine). The synthesis of lipid-poly(L-Glu)-(D-mannosamine)-(ethanolamine) was similar to that of lipid-poly(L-Glu)-(D-mannosamine), and the D-mannosamine and ethanolamine groups were conjugated onto the side chain (Scheme 5). Using lipid-poly(L-Glu)₄₀ as a representative example, the success of D-mannosamine and ethanolamine functionalization was confirmed by the appearance of peaks o (—CH— in D-mannosamine group), j (D-mannose-NH—), u (—CH₂ in ethanolamine group) and j (ethanol-NH—) at δ 6.45, 7.49, 3.12, 7.83 ppm, respectively (FIG. 12, FIG. 13, FIG. 14, FIG. 15, FIG. 16 and FIG. 17). The contents of D-mannosamine and ethanolamine groups in lipid-poly(L-Glu)-(D-mannosamine)-(ethanolamine) were determined by the integration area under peaks a, g, o and v (peak a of the lipid; peak g of the polypeptide; peak o of the D-mannosamine; peak v of ethanolamine). By changing the feed ratio of D-mannosamine and ethanolamine, lipid-poly(L-Glu)-(D-mannosamine)-(ethanolamine) with various D-mannosamine and ethanolamine groups were obtained (Tables 6, 7). The maximum degree of functionalization in lipid-poly(L-Glu)-(D-mannosamine)-(ethanolamine) series was approximately 90%.

TABLE 6
Reactant feed ratio of lipid-poly(L-
Glu)-(D-mannosamine)-(ethanolamine)

| | Polymer name | | | | |
|---|---|---|---|---|---|
| | Sub- | | Sub- | Acti- | |
| | strate 1 | Substrate 2 | strate 3 | vator | Base |
| | Lipid-polypeptide | | | | |
| | lipid- | | | | |
| | poly(L- | D- | Ethanol- | | |
| | Glu) | mannosamine | amine | HBTU | DIPEA |
| | (mg) | (mmol) | (mmol) | (mmol) | (mmol) |
| LPP9 | 180 | 1.0 | 1.0 | 1.2 | 3.0 |
| LPP10 | 180 | 1.5 | 0.5 | 1.2 | 3.0 |
| LPP11 | 180 | 1.75 | 0.25 | 1.2 | 3.0 |
| LPP12 | 180 | 0 | 2.0 | 1.2 | 3.0 |
| LPP13 | 185 | 1.0 | 1.0 | 1.2 | 3.0 |
| LPP14 | 185 | 2.0 | 1.0 | 1.2 | 3.0 |
| LPP15 | 185 | 3.0 | 1.0 | 1.2 | 3.0 |

TABLE 7
Design, structure and characteristics of the lipid-poly(L-Glu)-(D-mannosamine)-(ethanolamine)

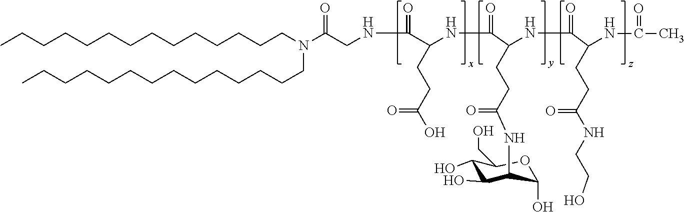

| | Structure units | | | | | |
|---|---|---|---|---|---|---|
| | x | y | z | | | |
| Lipid- | L- | L-Glu-D- | L-Glu- | | Mₙ^{NMR} | Functionalization |
| polypeptide | Glu | mannosamine | ethanolamine | n | [g mol⁻¹] | degree (%) |
| LPP9 | 5 | 8 | 27 | 40 | 8125.6 | 87.5 |
| LPP10 | 5 | 13 | 22 | 40 | 8716.0 | 87.5 |
| LPP11 | 6 | 16 | 18 | 40 | 9027.2 | 85.0 |
| LPP12 | 5 | 0 | 35 | 40 | 7180.9 | 87.5 |
| LPP13 | 6 | 10 | 24 | 40 | 8318.7 | 85.0 |
| LPP14 | 9 | 10 | 21 | 40 | 8189.5 | 77.5 |
| LPP15 | 8 | 12 | 20 | 40 | 8468.7 | 80.0 |

### Example 3: Size, PDI, and Zeta Potential of mRNA LNPs

Characteristics of the mRNA LNPs formulated manually with LPPs at varying mole ratios are summarized in Tables 8-13 and the mRNA LNPs prepared using the microfluidics device are displayed in Table 14. For all ALC-0159 formulations, the mole ratio of ALC-0159 is kept constant at 1.6%. Most formulations yielded particle sizes of around 200 nm or below. They also showed a narrow size distribution with PDI of around 0.2 or below. All formulations showed a near neutral zeta potential at <±10 mV. Formulations for LPP9, LPP10, LPP11, and LPP12 tend to show a smaller particle size and PDI despite the chain is length of the polypeptide due to the neutralization of charged subunits within the polypeptide. This would reduce charge repulsion and lead to smaller particles and PDI after formulation.

TABLE 8
Characteristics of manually formulated
mRNA LNPs at LPP mole ratio of 0.4%

| | Mole | Diameter | | Zeta potential |
|---|---|---|---|---|
| Formulation | ratio (%) | (nm) | PDI | (mV) |
| LPP1 | 0.4 | 178 ± 2 | 0.174 ± 0.011 | 0.98 ± 1.18 |
| LPP2 | | 166 ± 2 | 0.192 ± 0.011 | −0.49 ± 1.19 |
| LPP3 | | 157 ± 3 | 0.178 ± 0.018 | 0.40 ± 0.49 |

TABLE 9
Characteristics of manually formulated
mRNA LNPs at LPP mole ratio of 0.8%

| | Mole | Diameter | | Zeta potential |
|---|---|---|---|---|
| Formulation | ratio (%) | (nm) | PDI | (mV) |
| LPP1 | 0.8 | 313 ± 35 | 0.408 ± 0.107 | −1.19 ± 0.15 |
| LPP2 | | 420 ± 25 | 0.429 ± 0.078 | −2.74 ± 1.44 |
| LPP3 | | 272 ± 18 | 0.389 ± 0.061 | 0.03 ± 1.66 |

TABLE 10
Characteristics of manually formulated
mRNA LNPs at LPP mole ratio of 1.0%

| | Mole | Diameter | | Zeta potential |
|---|---|---|---|---|
| Formulation | ratio (%) | (nm) | PDI | (mV) |
| LPP2 | 1.0 | 153 ± 7 | 0.134 ± 0.083 | −4.57 ± 1.60 |
| LPP4 | | 118 ± 0 | 0.152 ± 0.014 | 1.23 ± 0.73 |
| LPP9 | | 112 ± 2 | 0.130 ± 0.014 | 4.23 ± 0.95 |
| LPP10 | | 100 ± 1 | 0.147 ± 0.005 | 1.95 ± 0.93 |
| LPP11 | | 103 ± 0 | 0.153 ± 0.019 | 3.73 ± 2.03 |
| LPP12 | | 199 ± 1 | 0.044 ± 0.029 | −4.54 ± 2.75 |

TABLE 11
Characteristics of manually formulated
mRNA LNPs at LPP mole ratio of 1.2%

| | Mole | Diameter | | ZetaA potential |
|---|---|---|---|---|
| Formulation | ratio (%) | (nm) | PDI | (mV) |
| LPP1 | 1.2 | 329 ± 13 | 0.418 ± 0.070 | −0.40 ± 0.89 |
| LPP2 | | 202 ± 5 | 0.341 ± 0.023 | −4.17 ± 0.34 |
| LPP3 | | 252 ± 26 | 0.317 ± 0.055 | −0.86 ± 0.31 |
| LPP4 | | 139 ± 1 | 0.170 ± 0.024 | 0.88 ± 1.00 |
| LPP8 | | 123 ± 2 | 0.12 ± 0.03 | 1.51 ± 0.84 |
| LPP9 | | 110 ± 1 | 0.155 ± 0.030 | 2.32 ± 2.75 |
| LPP10 | | 108 ± 1 | 0.192 ± 0.019 | 1.75 ± 0.50 |
| LPP11 | | 106 ± 1 | 0.212 ± 0.014 | 3.32 ± 0.93 |
| LPP12 | | 210 ± 2 | 0.0457 ± 0.020 | −6.25 ± 0.88 |

TABLE 12
Characteristics of manually formulated
mRNA LNPs at LPP mole ratio of 1.4%

| | Mole | Diameter | | Zeta potential |
|---|---|---|---|---|
| Formulation | ratio (%) | (nm) | PDI | (mV) |
| LPP2 | 1.4 | 189 ± 15 | 0.277 ± 0.066 | −4.34 ± 0.80 |
| LPP4 | | 137 ± 3 | 0.186 ± 0.009 | 1.09 ± 1.17 |
| LPP8 | | 129 ± 3 | 0.19 ± 0.01 | −0.28 ± 1.63 |
| LPP9 | | 116 ± 1 | 0.132 ± 0.032 | 2.75 ± 1.91 |
| LPP10 | | 113 ± 2 | 0.199 ± 0.014 | 1.38 ± 0.84 |
| LPP11 | | 107 ± 0 | 0.195 ± 0.024 | 1.61 ± 1.83 |
| LPP12 | | 217 ± 4 | 0.0773 ± 0.026 | −6.01 ± 0.13 |

TABLE 13
Characteristics of manually formulated
mRNA LNPs at LPP mole ratio of 1.6%.

| | Mole | Diameter | | Zeta potential |
|---|---|---|---|---|
| Formulation | ratio (%) | (nm) | PDI | (mV) |
| ALC-0159 | 1.6 | 104 ± 1 | 0.140 ± 0.021 | 0.79 ± 2.41 |
| LPP1 | | 182 ± 5 | 0.139 ± 0.007 | −0.17 ± 2.45 |
| LPP2 | | 171 ± 4 | 0.206 ± 0.036 | −2.13 ± 2.83 |
| LPP3 | | 147 ± 3 | 0.148 ± 0.011 | −0.76 ± 0.17 |
| LPP4 | | 140 ± 2 | 0.194 ± 0.012 | 0.63 ± 0.52 |
| LPP8 | | 380 ± 7 | 0.36 ± 0.07 | 1.99 ± 0.69 |
| LPP9 | | 104 ± 1 | 0.147 ± 0.014 | 4.01 ± 1.15 |
| LPP10 | | 107 ± 1 | 0.156 ± 0.008 | 2.57 ± 1.35 |
| LPP11 | | 109 ± 0 | 0.146 ± 0.003 | 1.55 ± 1.40 |
| LPP12 | | 199 ± 1 | 0.059 ± 0.020 | −5.70 ± 0.32 |

TABLE 14
Characteristics of mRNA LNPs formulated
through microfluidics device

| | Mole ratio | Diameter | | Zeta potential |
|---|---|---|---|---|
| Formulation | (%) | (nm) | PDI | (mV) |
| ALC-0159 | 1.6 | 57 ± 1 | 0.133 ± 0.008 | 0.16 ± 1.71 |
| LPP2 | 1.2 | 204 ± 2 | 0.137 ± 0.035 | −4.90 ± 1.59 |

### Example 4: Encapsulation Efficiencies of mRNA LNPs

To ensure the efficient delivery of the nucleic acid cargo into the targeted cells, the mRNA LNPs formulated need to have a sufficient encapsulation efficiency that would indicate an adequate amount of mRNA encapsulated into the LNPs to be transfected into cells. Encapsulation efficiencies were determined through the RiboGreen RNA assay and calculated. The encapsulation efficiencies of manually formulated mRNA LNPs are shown in Tables 15-20 and the encapsulation efficiencies of mRNA LNPs made through the microfluidics device are displayed in Table 21. Except for LPP1 and LPP3 formulations, and LPP2 0.4% and LPP2 0.8%, all other formulations showed a greater encapsulation efficiency than ALC-0159 (17.5%) when formulated manually. LPP12 showed exceptionally high encapsulation efficiencies at >80%, possibly due to the high percentage of neutralized charged subunits in the LPP12 polymer, reducing the repulsion between charged subunits and competition between the charged groups in the polypeptides and mRNA for binding the ionizable lipid, and thus allowing for better encapsulation of mRNA. Both ALC-0159 and LPP2 formulations made through the microfluidics device yielded a high encapsulation efficiency>80%, which is to be expected due to the controlled precision of mixing the organic and lipid phases together through the device, allowing for better encapsulation when compared to manually formulated mRNA LNPs. These results indicate that LPPs were able to act as a suitable replacement for ALC-0159 with regards to encapsulating mRNAs in LNPs.

TABLE 15
Encapsulation efficiencies of manually formulated
mRNA LNPs at mole ratio of 0.4%.

| Lipid | Mole ratio (%) | Encapsulation efficiency (%) |
|---|---|---|
| LPP1 | 0.4 | −1.1 ± 2.9 |
| LPP2 | | 7.4 ± 1.1 |
| LPP3 | | 4.7 ± 13.5 |

TABLE 16
Encapsulation efficiencies of manually formulated
mRNA LNPs at mole ratio of 0.8%.

| Lipid | Mole ratio (%) | Encapsulation efficiency (%) |
|---|---|---|
| LPP1 | 0.8 | 11.5 ± 18.0 |
| LPP2 | | 10.9 ± 8.6 |
| LPP3 | | 1.6 ± 7.7 |

TABLE 17
Encapsulation efficiencies of manually formulated
mRNA LNPs at mole ratio of 1.0%.

| Lipid | Mole ratio (%) | Encapsulation efficiency (%) |
|---|---|---|
| LPP2 | 1.0 | 64.7 ± 0.3 |
| LPP4 | | 39.0 ± 4.1 |
| LPP9 | | 37.8 ± 0.4 |
| LPP10 | | 56.1 ± 2.5 |
| LPP11 | | 22.1 ± 0.3 |
| LPP12 | | 84.1 ± 0.1 |

TABLE 18
Encapsulation efficiencies of manually formulated
mRNA LNPs at mole ratio of 1.2%.

| Lipid | Mole ratio (%) | Encapsulation efficiency (%) |
|---|---|---|
| LPP1 | 1.2 | −3.4 ± 10.3 |
| LPP2 | | 65.0 ± 1.0 |
| LPP3 | | −3.1 ± 11.8 |
| LPP4 | | 41.1 ± 2.8 |
| LPP8 | | 54.5 ± 8.0 |
| LPP9 | | 38.0 ± 3.5 |
| LPP10 | | 42.9 ± 0.0 |
| LPP11 | | 39.2 ± 1.0 |
| LPP12 | | 86.9 ± 0.3 |

TABLE 19
Encapsulation efficiencies of manually formulated
mRNA LNPs at mole ratio of 1.4%.

| Lipid | Mole ratio (%) | Encapsulation efficiency (%) |
|---|---|---|
| LPP2 | 1.4 | 61.8 ± 1.2 |
| LPP4 | | 42.4 ± 2.5 |
| LPP8 | | 73.9 ± 4.9 |
| LPP9 | | 43.7 ± 1.0 |
| LPP10 | | 35.8 ± 2.0 |
| LPP11 | | 26.4 ± 2.1 |
| LPP12 | | 83.3 ± 0.7 |

TABLE 20
Encapsulation efficiencies of manually formulated
mRNA LNPs at mole ratio of 1.6%.

| Lipid | Mole ratio (%) | Encapsulation efficiency (%) |
|---|---|---|
| ALC-0159 | 1.6 | 17.5 ± 6.0 |
| LPP1 | | 7.5 ± 0.7 |
| LPP2 | | 68.2 ± 1.8 |
| LPP3 | | 3.6 ± 10.3 |
| LPP4 | | 46.7 ± 2.5 |
| LPP8 | | 69.0 ± 5.1 |
| LPP9 | | 39.9 ± 1.4 |
| LPP10 | | 28.2 ± 3.5 |
| LPP11 | | 26.0 ± 1.4 |
| LPP12 | | 80.8 ± 1.3 |

TABLE 21
Encapsulation efficiencies of mRNA LNPs
formulated through microfluidics device.

| Lipid | Mole ratio (%) | Encapsulation efficiency (%) |
|---|---|---|
| ALC-0159 | 1.6 | 88.0 ± 0.6 |
| LPP2 | 1.2 | 84.1 ± 2.1 |
| LPP8 | 1.2 | 92.4 ± 1.6 |

The formulations have a nano-size (LPP8: 80±1) and narrow size distribution (LPP8: PDI=0.09±0.01), neutral surface (LPP8: zeta potential=−2.45±1.43 mV) and high encapsulation efficiency, ideal for in vivo applications.

### Example 5: In Vitro Cell Viability and Transfection Efficiency of mRNA LNPs In HELA Cells

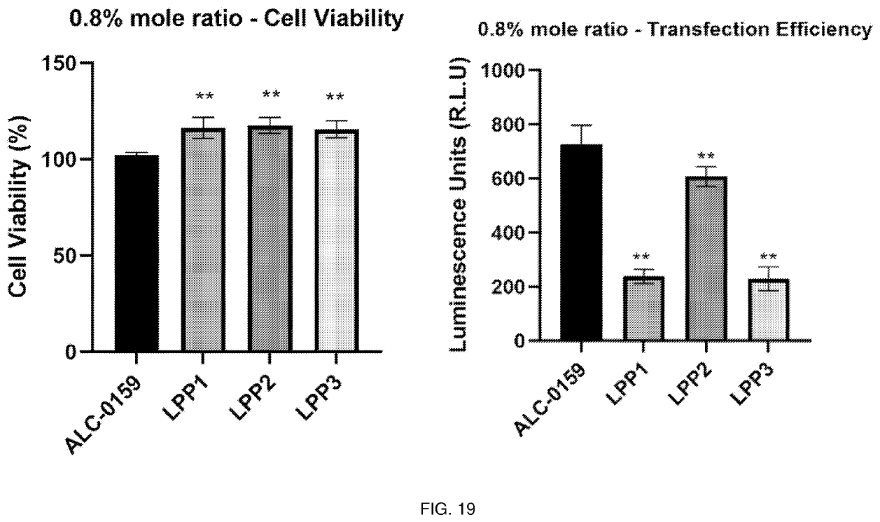

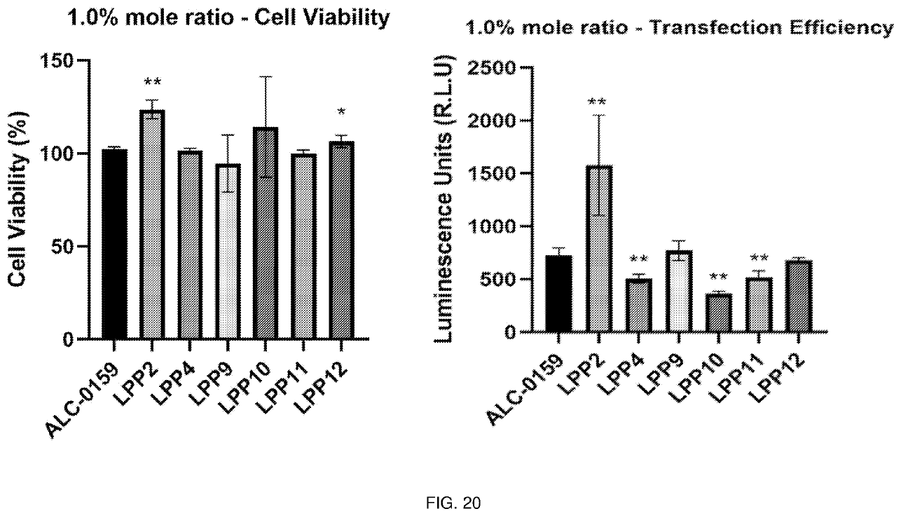

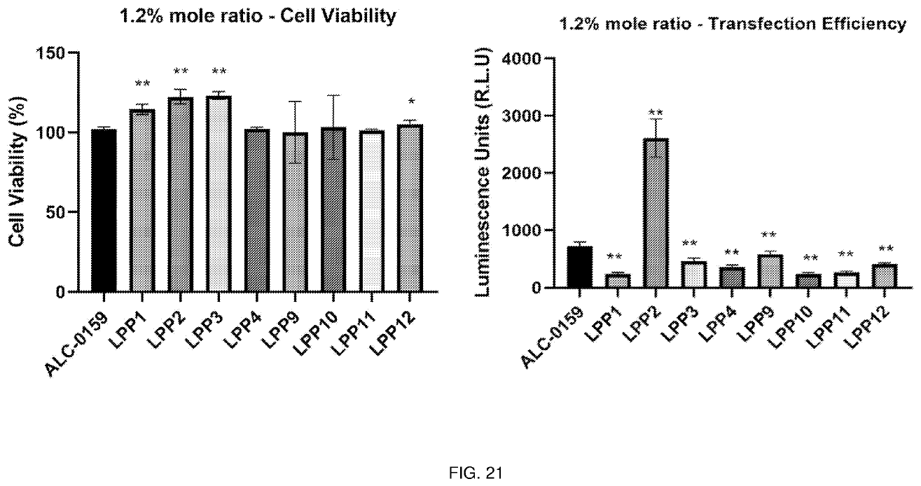

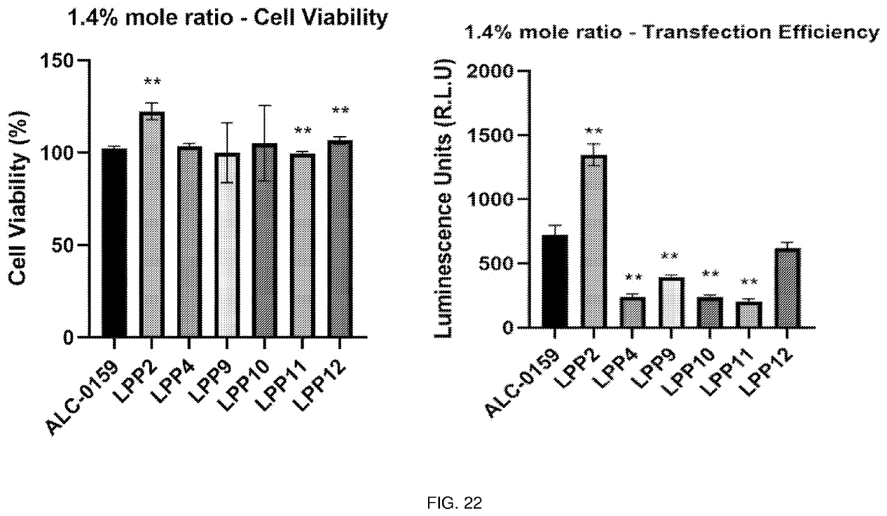

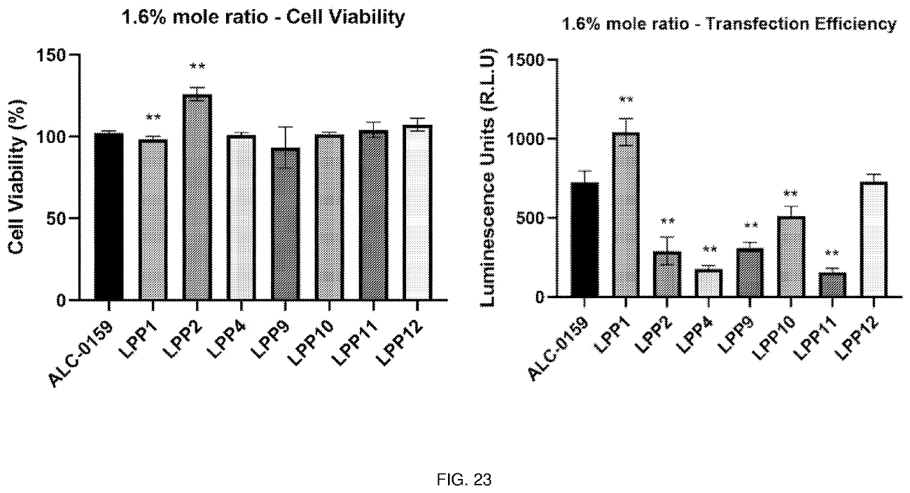

To determine the efficacy of LPPs as a replacement for ALC-0159 with regards to the transfection of cells, HELA cells were dosed with Fluc mRNA LNPs formulated manually to determine mRNA transfection efficiency and cell viability. As displayed in FIG. 19, FIG. 20, FIG. 21, FIG. 22 and FIG. 23, like ALC-0159, all LPP formulations showed negligible cytotoxicity. LPP2, except at mole ratio 1.6%, showed significantly higher mRNA transfection efficiency than ALC-0159. LPP2, which has a shorter subunit chain length compared to the other LPPs, yielded a greater transfection efficiency. However, LPP4 (24 subunits) which is shorter than LPP2 (36 subunits) had poorer transfection results, indicating that there is a hydrophilicity/hydrophobicity balance for LPPs to obtain optimal transfection efficiency. LPP2 mRNA LNPs at 1.2% mole content showed higher transfection efficiency than the rest LPPs, and were thus selected for in vivo transfection study.

### Example 6: In Vitro Cell Viability and Transfection Efficiency of mRNA LNPs in RAW264.7 cells

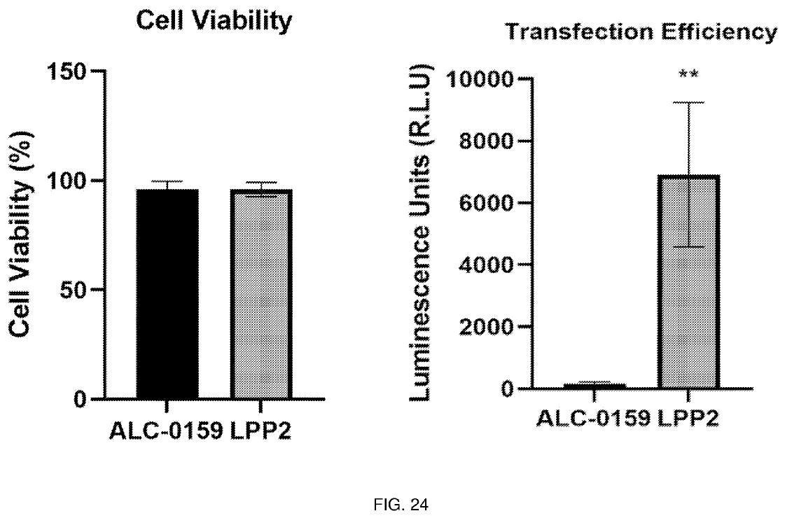

To assess the effectiveness of mRNA delivery using mannose-functionalized PEG-free LNPs, mRNA LPP2 LNPs made at mole ratio 1.2% of LPP2 were used to transfect RAW264.7 cells at a dose of 100 ng of mRNA per well. As observed from FIG. 24, both ALC-0159 and LPP2 showed minimal cytotoxicity in RAW264.7 cells. Furthermore, LPP2 yielded a much greater transfection efficiency in RAW264.7 cells compared to ALC-0159, indicating the capability of mannose in the LPP to target mannose receptors in RAW264.7 cells and to induce greater transfection efficiency.

### Example 7: In Vivo mRNA Transfection of mRNA LNPs

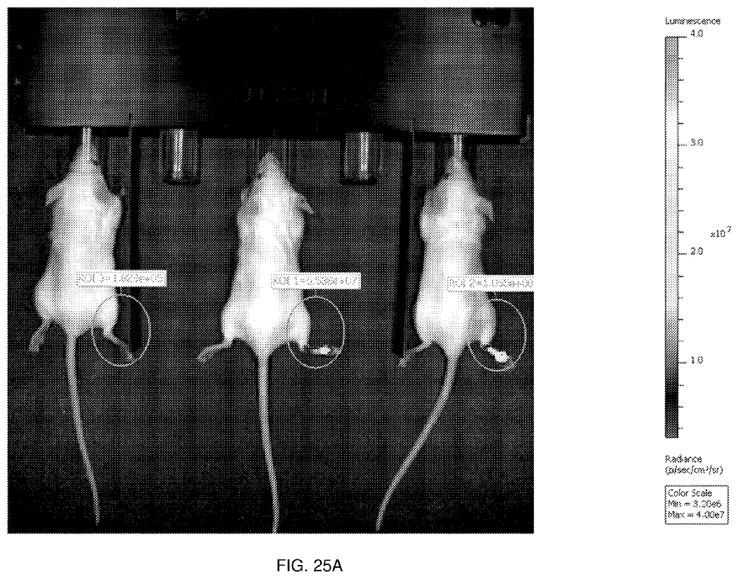

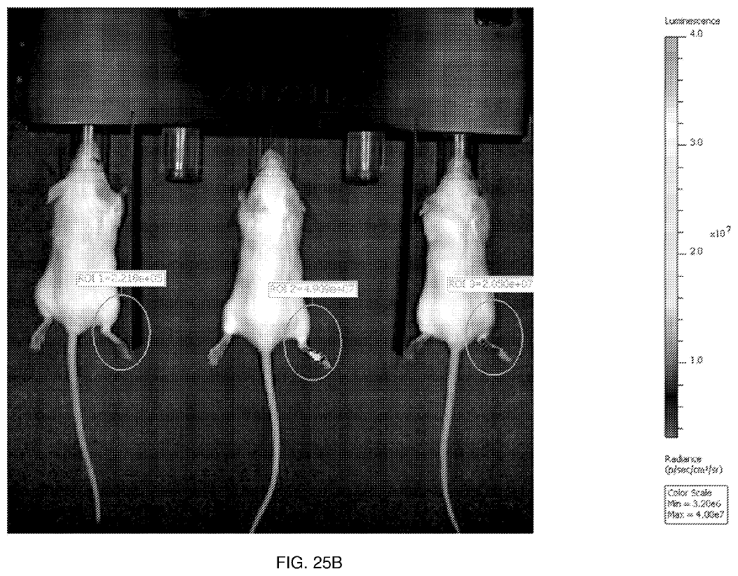

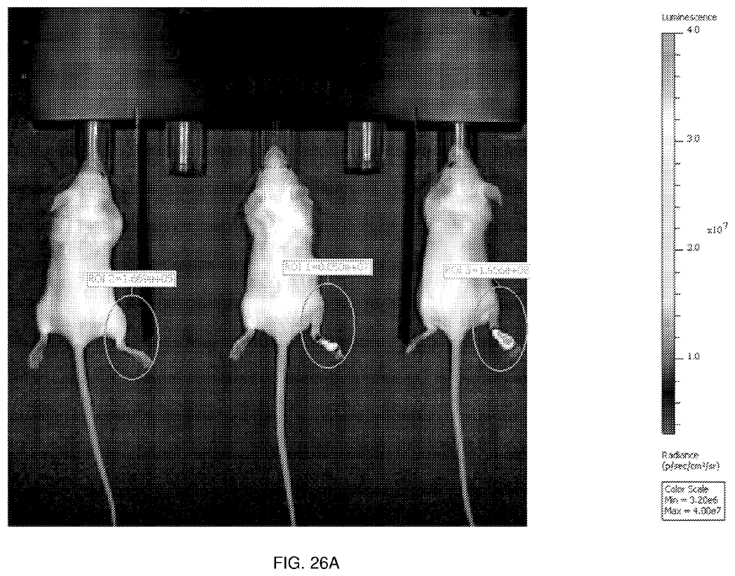

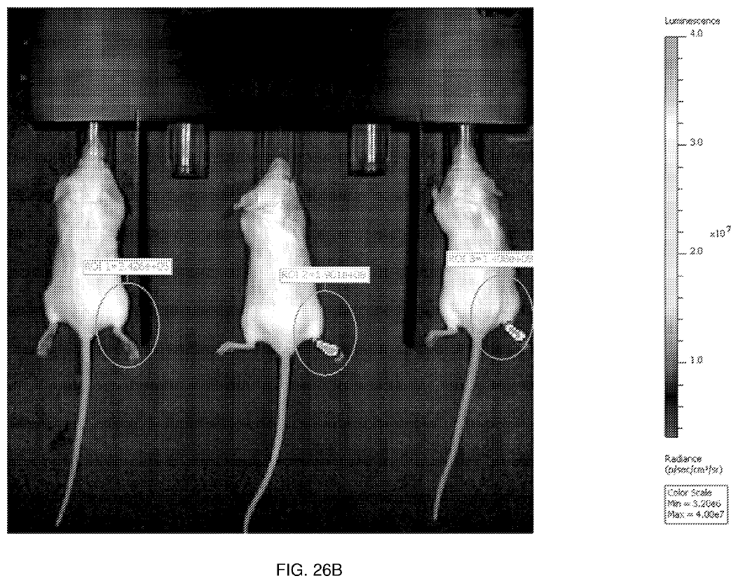

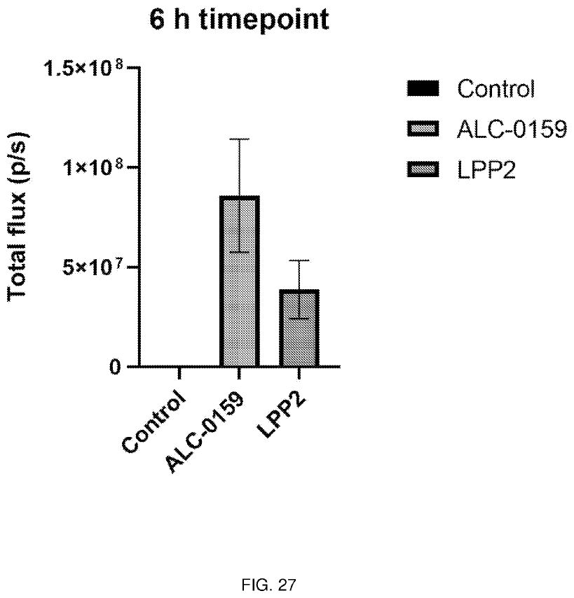

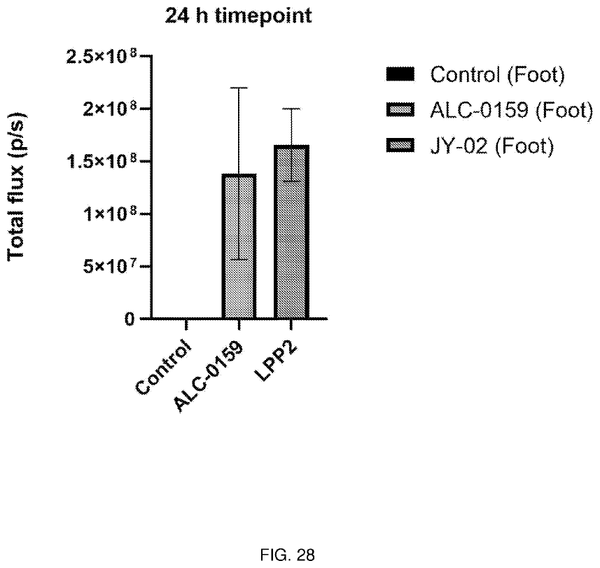

For in vivo analysis of transfection efficiency, mRNA LPP2 LNPs at 1.2% mole content of LPP2 were formulated using the microfluidics device and compared against mRNA ALC-0159 LNPs. Both ALC-0159 and LPP2 mRNA LNPs were introduced into the mice through the subcutaneous route at the right foot. At 6 h post injection, the dorsal view of the mice was imaged to determine luminescence intensity after incubation for 10 min with D-luciferin injected through the intraperitoneal route and displayed in FIG. 25A and FIG. 25B. At 24 h post injection, the luminescence intensity of the mice was determined and displayed in FIG. 26A and FIG. 26B. The total flux of the regions of interest at an exposure time of 10 s are displayed in Table 22 and FIG. 27 for the 6 h time point and Table 23 and FIG. 28 for the 24 h time point. At both the 6 h and 24 h time points, transfection efficiency of LPP2, determined through total flux measured, was comparable to that of ALC-0159.

TABLE 22
Total flux of the regions of interest at the right
foot for the 6 h time point from the dorsal view.
Values were obtained from exposure time of 10 s

| Group | Average total flux (p/s) |
|---|---|
| Control | 2.02E+05 |
| ALC-0159 | 8.60E+07 |
| LPP2 | 3.88E+07 |

TABLE 23
Total flux of the regions of interest at the right
foot for the 24 h time point from the dorsal view.
Values were obtained from exposure time of 10 s

| Group | Average total flux (p/s) |
|---|---|
| Control | 2.55E+05 |
| ALC-0159 | 1.38E+08 |
| LPP2 | 1.66E+08 |

### Example 8: Summary

mRNA LNPs encapsulated using the LPPs as a replacement for ALC-0159 showed a good size distribution (PDI<0.2) and greater encapsulation efficiencies compared to LNPs formulated with ALC-0159 that is used in Pfizer/BioNTech mRNA LNP vaccine. Transfection efficiencies of mRNA LNPs formulated with LPP2 at a mole content of 1.2% were tremendously higher than mRNA LNPs formulated with ALC-0159 especially in macrophages. Through an in vivo study, mRNA LPP2 LNPs showed comparable transfection efficiency to mRNA ALC-0159 LNPs. Taken together, these results show that the LPPs can serve as a viable replacement for ALC-0159 in the delivery of mRNA, potentially reducing the risk of allergic reaction by the PEG-conjugated lipids and prolonging the plasma half-life of mRNA LNPs. Commercial use of mRNA LNPs formulated with LPPs may provide safer nanocarriers of mRNA vaccines that can mitigate allergic reactions observed in the current mRNA vaccines.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A compound represented by general formula (1) for preparing lipid nanoparticles encapsulating a therapeutic, prophylactic and/or biological agent: 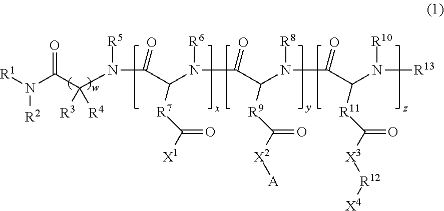
wherein
A comprises a hydrophilic moiety selected from carbohydrate/sugar/saccharide and derivatives thereof;
X¹ is —OR^{a} or —NR^{b}R^{c};
X⁴ is —OR^{y}, —SR^{z} or a hydrophilic group, where R^{y} to R^{z} are independently selected from the group consisting of H, optionally substituted alkyl, optionally substituted alkenyl or optionally substituted alkynyl;
X² and X³ are each independently —O— or —NR^{d}—, where R^{a} to R^{d} are independently selected from the group consisting of H, optionally substituted alkyl, optionally substituted alkenyl or optionally substituted alkynyl;
R¹ and R² are each independently a hydrophobic group;
R³, R⁴, R⁵, R⁶, R^{a} and R¹⁰ are each independently H, optionally substituted alkyl, optionally substituted alkenyl or optionally substituted alkynyl;
R⁷, R⁹, R¹¹ and R¹² are each independently optionally substituted alkyl, optionally substituted alkenyl or optionally substituted alkynyl;
R¹³ is H, optionally substituted alkyl, optionally substituted alkenyl or optionally substituted alkynyl, or —C(═O)R¹⁴, where R¹⁴ is optionally substituted alkyl, optionally substituted alkenyl, optionally substituted alkynyl;
w≥>1;
x is 0 or ≥1;
y is 0 or ≥1; and
z is 0 or ≥1.

2. The compound of claim 1, wherein the compound is capable of targeting carbohydrate receptors on a cell surface.

3. The compound of claim 1, wherein A is a 5-membered ring or represented by general formula (2) having a 6-membered ring structure: 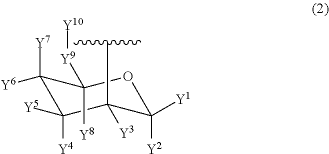
wherein
Y¹ to Y⁸ and Y¹⁰ are each independently selected from —H or —OH; and
Y⁹ is alkyl.

4. The compound of claim 1, wherein A is represented by general formula (2A) and/or (2B) having a 6-membered ring structure: 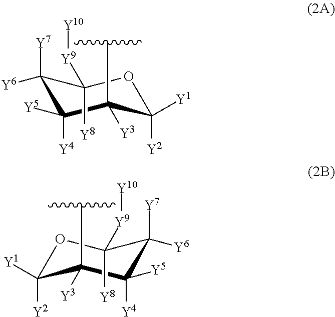
wherein
Y¹ to Y⁸ and Y¹⁰ are each independently selected from —H or —OH; and
Y⁹ is alkyl.

5. The compound of claim 1, wherein the total sum of x+y+z is from 1 to 300.

6. The compound of claim 1, wherein the hydrophobic group at R¹ and R² each independently comprises optionally substituted alkyl.

7. The compound of claim 1, wherein the compound is selected from the group consisting of LPP1, LPP2, LPP3, LPP4, LPP5, LPP6, LPP7, LPP8, LPP9, LPP10, LPP11, LPP12, LPP13, LPP14, LPP15 and combinations thereof.

8-10. (canceled)

11. A nanoparticle composition for delivery of a therapeutic, prophylactic and/or biological agent, the nanoparticle composition comprising:
a compound as claimed in claim 1; and
a therapeutic, prophylactic and/or biological agent that is encapsulated in said composition.

12. The nanoparticle composition of claim 11, wherein the composition further comprises:
(a) ionizable lipid;
(b) helper lipid; and
(c) sterol.

13. The nanoparticle composition of claim 12, wherein the ionizable lipid, helper lipid, sterol, and compound represented by general formula (1) are mixed at a mole ratio of 15-140:3-30:14-130:0.2-40.

14. The nanoparticle composition of claim 12, wherein the ionizable lipid is selected from ALC-0315, SM-102, Lipid 5, DLinDMA, D-Lin-MC2-DMA, DLin-MC3-DMA, D-Lin-MC4-DMA, Dlin-KC2-DMA, YSK05, AA3-Dlin, SSPalmM, SSPalmO-Phe, Lipid A9, L319, DODMA, CL1, BP Lipid 310, ATX-001, ATX-100, Lipid 2, 80-016B, BP Lipid 309, BP Lipid 307, 93-017S, 93-0170, NT1-014B, 306-012B-3, 306-012B, 113-016B, 3060i10, 3060i9-cis2, BAMEA-016B, Al-28, 113-012B, 98N12-5, Ckk-E12, OF-02, C12-200, BP Lipid 311, BP Lipid 308, BP Lipid 314, BP Lipid 312, LP01, TCL053, Lipid C24, BP Lipid 315, Lipid 29, 9A1P9, C13-112-tri-tail, C13-113-tri-tail, C13-112-tetra-tail, or C13-113-tetra-tail, C12-200 and combinations thereof.

15. The nanoparticle composition of claim 12, wherein the helper lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin and combinations thereof.

16. The nanoparticle composition of claim 12, wherein the sterol is selected from cholesterol, fecosterol, sitosterol, ergosterol, campesterol, stigmasterol, brassicasterol, avenasterol and combinations thereof.

17. The nanoparticle composition of claim 11, wherein the nanoparticle composition comprises nanoparticles having a N/P ratio from 2:1 to 40:1.

18. The nanoparticle composition of claim 11, wherein the nanoparticle composition comprises nanoparticles having an average particle size of no more than 800 nm.

19. The nanoparticle composition of claim 11, wherein the nanoparticle composition comprises nanoparticles having a zeta potential of from −40 mV to +40 mV.

20-22. (canceled)

23. A method of treating or preventing a disease, disorder or condition in a subject in need thereof, the method comprising administering a therapeutically effective amount of the nanoparticle composition as claimed in claim 11 to the subject.

24. The method of claim 23, wherein an immune response in the subject is to be induced through the administration of the nanoparticle composition thereto.

25. The method of claim 23, wherein the disease, disorder or condition is mediated by a coronavirus.

26. The method of claim 25, wherein the coronavirus is a SARS-CoV-2 coronavirus.
